(19) United States
(12) Patent Application Publication
(10) Pub. No.: US 2018/0303928 A1
(43) Pub. Date: Oct. 25, 2018

(54) H7N2 INFLUENZA A VIRUS

(71) Applicants: Sandra Newbury, Madison, WI (US); Kathy Lynn Toohey-Kurth, Redlands, CA (US); Francine Kay Cigel, Madison, WI (US)

(72) Inventors: Sandra Newbury, Madison, WI (US); Kathy Lynn Toohey-Kurth, Redlands, CA (US); Francine Kay Cigel, Madison, WI (US)

(21) Appl. No.: 15/914,744

(22) Filed: Mar. 7, 2018

Related U.S. Application Data
(60) Provisional application No. 62/468,101, filed on Mar. 7, 2017.
(60) Provisional application No. 62/469,970, filed on Mar. 10, 2017.

(51) Int. Cl. A61K 39/145; A61K 38/16; A61K 9/00; A61P 31/16

(52) U.S. Cl. CPC A61K 39/145; A61K 38/162; A61K 2039/70; A61K 9/0053; A61P 31/16; A61K 9/0019

(57) ABSTRACT

The invention provides an isolated H7 influenza A virus, as well as methods of preparing and using the virus, and genes or proteins thereof.

### CROSS-REFERENCE TO RELATED APPLICATIONS

This application claims the benefit of the filing date of U.S. application Ser. No. 62/469,970, filed on Mar. 10, 2017, and U.S. application Ser. No. 62/468,101, filed on Mar. 7, 2017, the disclosures of which are incorporated by reference herein.

### GOVERNMENT SUPPORT

This invention was made with government support under 2016-37620-25781 awarded by the NSDA/NIFA. The government has certain rights in the invention.

### BACKGROUND

Influenza is a major respiratory disease in some mammals and is responsible for substantial morbidity and economic losses each year. In addition, influenza virus infections can cause severe systemic disease, leading to death. The segmented nature of the influenza virus genome allows for reassortment of segments during virus replication in cells infected with two or more influenza viruses. The reassortment of segments, combined with genetic mutation and drift, can give rise to a myriad of divergent strains of influenza virus over time. The new strains exhibit antigenic variation in their hemagglutinin (HA) and/or neuraminidase (NA) proteins. The predominant current practice for the prevention of flu is vaccination. Most commonly, whole virus vaccines are used. The isolation of influenza virus and the identification and characterization of the HA and NA antigens in viruses associated with recent outbreaks is important for vaccine production. Based on prevalence and prediction, a vaccine is designed to stimulate a protective immune response against the predominant and expected influenza virus strains.

There are three general types of influenza viruses, Type A, Type B and Type C, which are defined by the absence of serological cross reactivity between their internal proteins. Influenza Type A viruses are further classified into subtypes based on antigenic and genetic differences of their glycoproteins, the HA and NA proteins. Aquatic birds are thought to act as a natural reservoir for influenza.

Although avian influenza viruses predominantly bind α2-3-linked SA, and human influenza viruses preferentially bind to α2-6-linked SA, a growing number of human cases of avian influenza infection have been reported, including H5N1, H7N2, H7N3, N7N7, and H9N2 strains. Since 1996, H7 viruses of the North American lineage have been circulating in regional live bird markets, containing a 24-nucleotide deletion resulting in an eight amino acid deletion in the receptor-binding site (RBS) of HA. Recent human infections with H7 in North America have raised public health concerns as to how these viruses adapt to such a dramatic structural change while remaining one of the predominant circulating viral strains.

### SUMMARY

The invention provides isolated H7N2 influenza virus and methods of making and using the virus. A H7N2 virus isolate was obtained from felines that caused an outbreak of respiratory disease in domestic cats that had tested positive for influenza antigen. The isolate was not previously shown to be circulating in the United States. The isolate was identified from a feline nasal swab as influenza A and the sample was then typed as a N2 strain. The virus may be isolated in EEC, CRFK or MDBK cells.

In one embodiment, the isolated H7 influenza virus has a characteristic residue(s) at a plurality of positions in HA-1 including but not limited to positions 24, 36, 84, 86, 93, 104, 109, 125, 138, 151, 158, 177, 180, 183, 188, 203, 258, 269, 290 and/or 292, including any combination thereof. For example, the isolated H7 influenza virus has a characteristic residue(s) at position 125 and at one or more of positions 24, 84, 93, 104, 109, 138, 151, 180, 183, 188, 203, or 292, or any combination thereof, and/or the isolated H7 influenza virus has a characteristic residue(s) at position 183 and at one or more positions of 24, 84, 93, 104, 109, 125, 138, 151, 180, 203, or 292, or any combination thereo. In one embodiment, the isolated H7 influenza virus has a characteristic residue(s) at a plurality of positions including but not limited to positions 24, 93, 138, 151, or 292, or any combination thereof, and/or a plurality of positions 84, 104, 109, 125, 180, 183, 188, or 203, or any combination thereof. In one embodiment, the isolated H7 influenza virus has a characteristic residue(s) at a plurality of positions including but not limited to positions 84, 104, 109, 125, 151, 158, 180, 183, 188, 203, 269, 290, and/or 292, e.g., one or more of 84, 104, 109, 125, 180, 183, 188, or 203, and one or more of 158, 269, or 290, or any combination thereof. In one embodiment, the isolated H7 influenza virus has a characteristic residue(s) at a plurality of positions including but not limited to positions 84, 104, 109, 125, 151, 180, 183, 188, 203, or any combination thereof and/or 292, e.g., position 125 and at three, four, five or more of positions 84, 104, 109, 151, 180, 183, 188, 203, or 292. In one embodiment, isolated H7 influenza virus comprises a viral HA segment with sequences for a HA-1 having greater than 92%, 95%, 96%, or 98% amino acid sequence identity to HA-1 encoded by a nucleotide sequence having any one of SEQ ID Nos. 44-52 or 54-64, e.g., any one of SEQ ID Nos. 45-52 or 54-64, or encoding an HA with greater than 92%, 95%, 96%, 97%, 98% or 99%, amino acid sequence identity to one of SEQ ID Nos. 125, 137, 149, 161, or 173.

For example, the isolated H7 virus has a characteristic residue in HA-1 at position 84 that is not T (threonine), at position 104 that is not G (glycine) or R (arginine), at position 109 that is not G, D or S (serine), at position 125 that is not A (alanine) or T, at position 180 that is not S or T, at position 183 is not T, at position 188 is not S, at position 203 is not 5, or any combination thereof. For example, the isolated H7 virus has a characteristic residue in HA-1 at position 84 that is N (asparagine) or Q (glutamine), at position 104 that is K (lysine) or H (histidine), at position 109 that is N or E, at position 125 that is 5, at position 151 is L (leucine), at position 180 that is N or Q, at position 183 is I (isoleucine), L or G, at position 188 is N or Q, at position 203 that is P (proline), or any combination thereof. In one embodiment, isolated H7 influenza virus comprises a viral HA segment with sequences for a HA-i having greater than 92%, 95%, 96%, or 98% amino acid sequence identity to HA-1 encoded by a nucleotide sequence having one of SEQ ID Nos. 44-52, 54-64, 85, 93, 101, 109, or 117, of a HA having greater than 92%, 93%, 94%, 95%, 96%, 97%, 98%, or 99%, amino acid sequence identity to one of SEQ ID Nos. 78, 125, 137, 149, 161, or 173. Optionally, the isolated virus also has a residue at position 24, 36, 86, 93, 138, 151, 158, 177, 258, 269, 292, or any combination that is serine, alanine, valine, isoleucine, glycine or threonine, and/or a residue at position 290 that is proline, serine, alanine, valine, isoleucine, glycine or threonine, or any combination thereof.

Other positions in HA-1 relative to the HA-1 encoded by a nucleotide sequence having one of SEQ ID Nos. 44-52, 54-64, 85, 93, 101, 109, or 117 may have conservative amino acid substitutions. Conservative refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In one embodiment, conservative amino acid substitution groups are: threonine-valine-leucine-isoleucine-alanine; phenylalanine-tyrosine; lysine-arginine; alanine-valine; glutamic-aspartic; and asparagine-glutamine.

In one embodiment, the isolated H7 influenza virus includes one or more viral proteins (polypeptides) having substantially the same amino acid sequence as one encoded by a nucleotide sequence comprising any one of SEQ ID Nos:1-18 and 40 (NA) (and NA sequences in FIGS. 6 and 14), encoded by a nucleotide sequence comprising any one of SEQ ID Nos. 44-52, 54-64, 85, 93, 101, 109, or 117 (HA), or encoded by a nucleotide sequence comprising any one of SEQ ID Nos. 19-26 (M) (and M sequences in FIGS. 6 and 14), and in one embodiment, so long as HA-1 has the characteristic residue(s). In one embodiment, the isolated H7 influenza virus includes a HA that is related to HA from, for example, A/avian/NewYork/81746-5/00; a PB2 that is related to a PB2 from, for example, A/chicken/NewYork/Sg-00306/1998 or A/chicken/NewYork/Sg-00305/1998; a PB1 that is related to PB1 from, for example, A/chicken/NewYork/Sg-00396/2002, A/chicken/NewYork/Sg-00397/2002 or A/chicken/NewYork/Sg-00398/2002; a PA that is related to PA from, for example, A/chicken/PA/143586/2001; a NP that is related to a NP from, for example, A/chicken/NewYork/119256-7/01 or A/chicken/PA/143586/01; a M that is related to M from, for example, A/chicken/NewYork/Sg-00396/2002 or A/chicken/NewYork/Sg-00398/2002; NS that is related to NS from, for example, A/unknown/NewYork/85161/2000 or A/chicken/NewYork/85161/2000, or any combination thereof. An amino acid sequence which is substantially the same as a reference sequence has at least or greater than 92% 93%, 95%, e.g., 96%, 97%, 98% or 99%, amino acid sequence identity to that reference sequence, e.g., a HA having greater than 92%, 93%, 94%, 95%, 96%, 97%, 98%, or 99%, amino acid sequence identity to one of, for example, SEQ ID Nos. 125, 137, 149, 161, or 173, and may include sequences with deletions, e.g., those that result in a deleted viral protein having substantially the same activity or capable of being expressed at substantially the same level as the corresponding full-length, mature viral protein, insertions, e.g., those that result in a modified viral protein having substantially the same activity or capable of being expressed at substantially the same level as the corresponding full-length, mature viral protein, and/or substitutions, e.g., those that result in a viral protein having substantially the same activity or capable of being expressed at substantially the same level as the reference protein. In one embodiment, the one or more residues which are not identical to those in the reference sequence may be nonconservative substitutions which one or more substitutions do not substantially alter the expressed level or activity of the protein with the substitution(s), and/or the level of virus obtained from a cell infected with a virus having that protein. As used herein, “substantially the same expressed level or activity” includes a detectable protein level that is about 80%, 90% or more, the protein level, or a measurable activity that is about 30%, 50%, 90%, e.g., up to 100% or more, the activity, of a full-length mature polypeptide including one encoded by a nucleotide sequence comprising any one of SEQ ID Nos:1-26, 40, 42, 44-52, 54-64, 85, 93, 101, 109, or 117 (and nucleotide sequences in FIGS. 6 and 14), or a HA having greater than 92%, 93%, 94%, 95%, 96%, 97%, 98%, or 99%, amino acid sequence identity to one of SEQ ID Nos. 125, 137, 149, 161, or 173. In one embodiment, the virus comprises a polypeptide with one or more, for instance, 2, 5, 10, 15, 20 or more, amino acid substitutions, e.g., conservative substitutions of up to 5%, 6%, 7%, 8%, 9% or 10%, of the residues of the full-length, mature form of a polypeptide encoded by a nucleotide sequence comprising any one of SEQ ID Nos: 1-26, 40, 42, or 44-52, 54-64, 85, 93, 101, 109, or 117, or has a HA having greater than 92%, 93%, 94%, 95%, 96%, 97%, 98%, or 99%, amino acid sequence identity to one of, for example, SEQ ID Nos. 125, 137, 149, 161, or 173. In one embodiment, the virus comprises a polypeptide with one or more, for instance, 2, 5, 10, 15, or 20, or more, amino acid substitutions, e.g., conservative substitutions of up to 2%, 3%, 4% 5%, or more, of the residues of, for example, a protein that is encoded by any one of SEQ ID Nos. 1-18 (NA) (and NA sequences in FIGS. 6 and 14), or 44-52, 54-64, 85, 93, 101, 109, or 117 (HA). The isolated virus may be employed alone or with one or more other virus isolates, other influenza virus isolates, in a vaccine, to raise virus-specific antisera, in gene therapy, and/or in diagnostics. Accordingly, the invention provides host cells infected with the virus, and isolated antibody specific for the virus.

The invention also provides an isolated nucleic acid molecule (polynucleotide) comprising a nucleic acid segment corresponding to at least one of the proteins of the H7 virus described herein, a portion of the nucleic acid segment for a viral protein having substantially the same level or activity as a corresponding polypeptide encoded by a nucleotide sequence having one of SEQ ID Nos: 1-26, 40, 42, 44-52, 54-64, 85, 93, 101, 109, or 117, or encoding a HA having greater than 92%, 93%, 94%, 95%, 96%, 97%, 98%, or 99%, amino acid sequence identity to one of SEQ ID Nos. 78, 125, 137, 149, 161, or 173, or the complement of the nucleic acid molecule. In one embodiment, the isolated nucleic acid molecule encodes a polypeptide which has substantially the same amino acid sequence, e.g., has at least (or greater than) 95%, e.g., 96%, 97%, 98% or 99%, contiguous amino acid sequence identity to a polypeptide encoded by a nucleotide sequence having one of SEQ ID Nos: 1-26, 40, 42, 44-52, 54-64, 85, 93, 101, 109, or 117, or encoding a HA having greater than 92%, 93%, 94%, 95%, 96%, 97%, 98%, or 99%, amino acid sequence identity to one of SEQ ID Nos. 78, 125, 137, 149, 161, or 173. In one embodiment, the isolated nucleic acid molecule comprises a nucleotide sequence which is substantially the same as, e.g., has at least 50%, e.g., 60%, 70%, 80% or 90% or more, contiguous nucleic acid sequence identity to, one of SEQ ID Nos: 1-26, 40, 42, 44-52, 54-64, 85, 93, 101, 109, or 117, or encoding a HA having greater than 92%, 93%, 94%, 95%, 96%, 97%, 98%, or 99%, amino acid sequence identity to one of SEQ ID Nos. 78, 125, 137, 149, 161, or 173, or the complement thereof, or encodes a polypeptide having at least (or greater than) 95%, e.g., 96%, 97%, 98% or 99%, contiguous amino acid sequence identity to a polypeptide encoded by a nucleotide sequence having one of SEQ ID Nos:1-26, 40, 42, 44-52, and 54-64. In one embodiment, the isolated nucleic acid molecule comprises sequences encoding a polypeptide that is encoded by a nucleotide sequence having one of SEQ ID Nos: 1-26, 40, 42, 44-52, 54-64, 85, 93, 101, 109, or 117, or a nucleic acid sequence with at least 95%, e.g., 96%, 97%, 98% or 99%, contiguous nucleic acid sequence identity thereto. In one embodiment, the isolated nucleic acid molecule comprises sequences encoding a polypeptide that has greater than 95%, e.g., 96%, 97%, 98% or 99%, contiguous amino acid sequence identity to one of SEQ ID Nos. 78, 125, 137, 149, 161, or 173.

The isolated nucleic acid molecule may be employed in a vector to express influenza proteins, e.g., for recombinant protein vaccine production or to raise antisera, as a nucleic acid vaccine, for use in diagnostics or, for vRNA production, to prepare chimeric genes, e.g., with other viral genes including other influenza virus genes, and/or to prepare recombinant virus. Thus, the invention also provides isolated viral polypeptides, recombinant virus, and host cells contacted with the nucleic acid molecule(s) and/or recombinant virus, as well as isolated virus-specific antibodies, for instance, obtained from mammals infected with the virus or immunized with an isolated viral polypeptide or polynucleotide encoding one or more viral polypeptides. Further provided is one or more isolated viral protein(s), e.g., for use as an immunogen, optionally in a virus-like particle (VLP).

The disclosure further provides at least one of the following isolated vectors, for instance, one or more isolated influenza virus vectors, or a composition comprising the one of a vector comprising a promoter operably linked to an influenza virus HA DNA for a HA comprising a sequence having substantially the same amino acid sequence as a protein that is encoded by a nucleotide sequence having one of SEQ ID Nos. 45-52, 54-64, 85, 93, 101, 109, or 117, 44-52, 54-64, 85, 93, 101, 109, or 117, or a HA having greater than 92%, 93%, 94%, 95%, 96%, 97%, 98%, or 99%, amino acid sequence identity to one of SEQ ID Nos. 125, 137, 149, 161, or 173, linked to a transcription termination sequence, or a vector comprising a promoter operably linked to an influenza virus NA DNA for a NA comprising a sequence having substantially the same amino acid sequence as a protein that is encoded by a nucleotide sequence having one of SEQ ID Nos. 1-18, 127, 135, 143, 151, or 159, or any one of linked to a transcription termination sequence. An influenza virus vector is one which includes at least 5′ and 3′ noncoding influenza virus sequences.

Hence, the disclosure provides vectors, e.g., plasmids, which encode influenza virus proteins, and/or encode influenza vRNA, both native and recombinant vRNA. Thus, a vector of the invention may encode an influenza virus protein (sense) or vRNA (antisense). Any suitable promoter or transcription termination sequence may be employed to express a protein or peptide, e.g., a viral protein or peptide, a protein or peptide of a nonviral pathogen, or a therapeutic protein or peptide. In one embodiment, to express vRNA, the promoter is a RNA polymerase I promoter, a RNA polymerase II promoter, a RNA polymerase III promoter, a T3 promoter or a T7 promoter. Optionally the vector comprises a transcription termination sequence such as a RNA polymerase I transcription termination sequence, a RNA polymerase II transcription termination sequence, a RNA polymerase III transcription termination sequence, or a ribozyme.

A composition of the invention may also comprise a gene or open reading frame of interest, e.g., a foreign gene encoding an immunogenic peptide or protein useful as a vaccine. Thus, another embodiment of the invention comprises a composition of the invention as described above in which one of the influenza virus genes in the vectors is replaced with a foreign gene, or the composition further comprises, in addition to all the influenza virus genes, a vector comprising a promoter linked to 5′ influenza virus sequences linked to a desired nucleic acid sequence, e.g., a cDNA of interest, linked to 3′ influenza virus sequences linked to a transcription termination sequence, which, when contacted with a host cell permissive for influenza virus replication optionally results in recombinant virus. In one embodiment, the DNA of interest is in an antisense orientation. The DNA of interest, whether in a vector for vRNA or protein production, may encode an immunogenic epitope, such as an epitope useful in a cancer therapy or vaccine, or a peptide or polypeptide useful in gene therapy.

A plurality of the vectors of the invention may be physically linked or each vector may be present on an individual plasmid or other, e.g., linear, nucleic acid delivery vehicle.

The disclosure also provides a method to prepare influenza virus. The method comprises contacting a cell, e.g., an avian or a mammalian cell, with the isolated virus or a plurality of the vectors of the invention, sequentially or simultaneously, for example, employing a composition comprising a plurality of the vectors, in an amount effective to yield infectious influenza virus. The disclosure also includes isolating virus from a cell infected with the virus or contacted with the vectors and/or composition. The disclosure further provides a host cell infected with the virus described herein or contacted with the composition or vectors described herein. In one embodiment, a host cell is infected with an attenuated (e.g., cold adapted) donor virus and a virus described herein to prepare a cold-adapted reassortant virus useful as a cold-adapted live virus vaccine.

The disclosure also provides a method to induce an immune response in a mammal, e.g., to immunize a mammal, against one more pathogens, e.g., against a virus as disclosed herein and optionally a bacteria, a different virus, or a parasite or other antigen. An immunological response to a composition or vaccine is the development in the host organism of a cellular and/or antibody-mediated immune response to a viral polypeptide, e.g., an administered viral preparation, polypeptide or one encoded by an administered nucleic acid molecule, which can prevent or inhibit infection to that virus or a closely (structurally) related virus. Usually, such a response consists of the subject producing antibodies, B cell, helper T cells, suppressor T cells, and/or cytotoxic T cells directed specifically to an antigen or antigens included in the composition or vaccine of interest. The method includes administering to the host organism, e.g., a mammal, an effective amount of the influenza virus of the invention, e.g., an attenuated, live virus, optionally in combination with an adjuvant and/or a carrier, e.g., in an amount effective to prevent or ameliorate infection of an animal such as a mammal by that virus or an antigenically closely related virus. In one embodiment, the virus is administered intramuscularly while in another embodiment, the virus is administered intranasally. In one embodiment, the virus is administered orally while in another embodiment, the virus is administered subcutaneously or ocularly. In some dosing protocols, all doses may be administered intramuscularly or intranasally, while in others a combination of intramuscular and intranasal administration is employed. The vaccine may further contain other isolates of influenza virus including recombinant influenza virus, other pathogen(s), additional biological agents or microbial components, e.g., to form a multivalent vaccine. In one embodiment, intranasal vaccination with inactivated influenza virus, e.g., H7N2 canine influenza virus and a mucosal adjuvant, e.g., the non-toxic B chain of cholera toxin, may induce virus-specific IgA and neutralizing antibody in the nasopharynx as well as serum IgG.

The influenza vaccine may employed with other anti-virals, e.g., amantadine, rimantadine, and/or neuraminidase inhibitors, e.g., the vaccine may be administered separately, for instance, administered before and/or after, or in conjunction with those anti-virals.

### BRIEF DESCRIPTION OF THE FIGURES

FIGS. 1A-G. Partial NA and M sequences of viral isolates described herein (N2 sequences are SEQ ID Nos. 1-18 and M sequences are SEQ ID Nos. 19-26).

FIGS. 2A-I. Alignment of N2 sequences from samples of A/feline/New York/16-040082-1/2016 (SEQ ID NO:40), CY014897 (an isolate from a bird, SEQ ID NO:41) and SEQ ID Nos. 1-18.

FIGS. 3A-D. Alignment of M sequences from A/feline/New York/16-040082-1/2016 (“NVDL”; SEQ ID NO:42), EU742905 (an isolate from a bird, SEQ ID NO:43) and SEQ ID Nos. 19-26.

FIGS. 4A-E. Alignment of HA sequences from 16-040082-1/2016 (SEQ ID NO:44), 16-040082 by WVDL (M16-3416Ig; SEQ ID NO:45), and other isolates (SEQ ID Nos. 46-52).

FIGS. 5A-E. Alignment of HA sequences from 16-040082 (SEQ ID NO:53) and other isolates (SEQ ID Nos. 54-64).

FIGS. 6A-K. Whole genome sequence of A/feline/New York/16-040082-1/2016 (nucleotide sequences are SEQ ID Nos. 65-72 and amino acid sequences are SEQ ID Nos. 73-84).

FIG. 7. Phylogenetic tree of influenza A viral hemagglutin segments from New York, N.Y., USA, compared with reference viruses. Phylogenetic analysis was performed for selected influenza A viruses representing major lineages. The evolutionary history was inferred using the neighbor-joining method (Saitou et al., 1987). The optimal tree with the branch length sum of 1.22521320 is shown. The percentage of replicate trees in which the associated taxa clustered together in the bootstrap test (500 replicates) is shown next to the branches (Felsenstein et al., 1985). The tree is drawn to scale, with branch lengths in the same units as those of the evolutionary distances used to infer the phylogenetic tree. The evolutionary distances were computed using the Tamura 3-parameter method (Tamura, 1992) and are in the units of the number of base substitutions per site. The analysis involved 44 nt sequences. Codon positions included were 1st+2nd+3rd+noncoding. All positions containing gaps and missing data were eliminated. The final dataset contained a total of 1,612 positions. Evolutionary analyses were conducted in MEGA7 (Kumar et al., 2016).

FIG. 8. Growth properties of A/feline/NY/16 and A/chicken/NY/99 influenza A(H7N2) viruses in mammalian and avian cells at different temperatures, New York, N.Y. USA. Cells were infected with viruses at a multiplicity of infection of 0.005 and incubated at 33° C. and 37° C. (or at 37° C. and 39° C. for avian CEF cells). Supernatants were harvested at the indicated time points. Virus titers were determined by use of plaque assays in Madin-Darby canine kidney (MDCK) cells. The species from which the cell lines are derived are shown. The values presented are the averages of 3 independent experiments±SD. Statistical significance was determined as described in the online Technical Appendix (https://wwwnc.cdc.gov/EID/article/24/1/17-1240-Techapp1.pdf). *p≤0.05; **p≤0.01; ***p≤0.001; ****p≤0.0001. A549, human lung carcinoma epithelial cells; Clone81, cat kidney fibroblast cells; Fc2Lu, cat lung cells; CEF, chicken embryo fibroblast cells.

FIG. 9. Body weight and temperature changes in cats infected with A/feline/NY/16 and A/chicken/NY/99 influenza A(H7N2) viruses, New York, N.Y., USA. Three cats per group were infected intranasally with 10⁶ PFU of viruses and monitored for bodyweight and temperature changes.

FIG. 10. Immunohistochemistry findings in cats infected with influenza A(H7N2) virus, New York, N.Y., USA. Shown are representative sections of nasal turbinates and lungs of cats infected with the indicated viruses on days 3 and 6 postinfection. Three cats per group were infected intranasally with 106 PFU of virus, and tissues were collected on days 3 and 6 postinfection. Type A influenza, virus nucleoprotein (NP) was detected by a mouse monoclonal antibody to this protein. For nasal turbinate sections: −, no NP-positive cells; +/−, NP-positive cells detected in 1-2 focal regions; +, NP-positive cells detected in >2 focal regions; 2+, NP-positive cells in large regions. For bronchus and alveolar sections: −, no NP-positive cells; +/−, ≥5 NP-positive cells; +, ≥6 NP-positive cells. NP-positive cells were detected in focal, but not in diffuse bronchial and alveolar sections. For all analyses, the entire sections were evaluated. Scale bars indicate 50 μm (nasal turbinates) or 100 μm (lung).

FIGS. 11A-D. Pathology findings in cats infected with A/feline/NY/16 influenza A(H7N2) virus on day 6 postinfection, New York, N.Y., USA. A) in lungs, moderately severe histopathologic changes are present in the lower airways. The lamina propria of bronchi (B) and bronchioles (Br) and the surrounding interstitium are infiltrated by numerous histiocytes, lymphocytes, and plasma cells (*), which also extend into and expand neighboring alveolar septa. The infiltrates extend into and expand nearby alveolar septa. The lumina of bronchioles are filled with numerous foamy macrophages, viable and degenerating neutrophils, proteinaceous fluid, and sloughed respiratory epithelial cells. Hyperplasia of bronchiole-associated lymphoid tissue (open arrow) and perivascular edema (solid arrow) are present. Scale bar indicates 500 μm. B) in nasal cavities, copious amounts of exudate are present comprising numerous degenerating and necrotic neutrophils, cellular debris, proteinaceous fluid, and strands of mucin. The respiratory epithelium covering the nasal turbinates (T) is extensively eroded. The underlying lamina propria appears diffusely bluish-purple due to infiltration by moderate-to-large numbers of histiocytes, neutrophils, lymphocytes, and plasma cells (*). Scale bar indicates 500 μm. C) In the trachea, a locally extensive focus of inflammation is present in the tracheal wall. Moderate numbers of histiocytes, lymphocytes, and plasma cells, and a few neutrophils, infiltrate the respiratory epithelium (RE), lamina propria, and submucosa. Submucosal glands (SG) are surrounded by the inflammatory infiltrates and effaced in the areas of heaviest infiltration (*). Tracheal cartilage (C). Scale bar indicates 100 μm. D) In the duodenum, inflammatory cell infiltrates (*) in the submucosa of the duodenum are present between and around Brunner's glands (BG). Scale bar indicates 100 μm.

FIGS. 12A-C. Receptor-binding specificities of influenza A viruses, New York, N.Y., USA. A) A representative human virus, A/Kawasaki/173-PR8(H1N1) is shown for comparison with B) the avian influenza A(H7N2) virus A/chicken/NY/99 and C) the feline influenza. A(H7N2) virus A/feline/NY/16. Receptor-binding specificities of the avian and feline viruses were compared with those of the human virus in a glycan microarray containing α2,3- and α2,6-linked sialosides. Error bars represent SDs calculated from 4 replicate spots of each glycan. RFU, relative fluorescence units.

FIG. 13. Distribution of α2,3- and α2,6-linked sialosides in the respiratory organs of a cat, New York, N.Y., USA. The α2,3- and α2,6-linked sialosides in the respiratory organs of a naïve cat were detected with biotinylated Maackia amurensis lectin I or II (MAA I, MAA II) or Sambucus nigra lectin (SNA I), respectively. Inset shows closer view of MAA III binding with alveolar epithelium in the lung. Plus signs (+) indicate that sialosides were detected. Scale bars indicate 50 μm.

FIGS. 14A-TT. WVDL sequences from isolate WVDL-3 (SEQ ID Nos. 85-92 for nucleotide sequences encoding SEQ ID Nos. 125-136), WVDL-9 (SEQ ID Nos. 93-100 for nucleotide sequences encoding SEQ ID Nos. 137-148), WVDL-14 (SEQ ID Nos. 101-108 for nucleotide sequences encoding SEQ ID Nos. 149-160), WVDL-16 (SEQ ID Nos. 109-116 for nucleotide sequences encoding SEQ ID Nos. 161-172), and WVDL-20 (SEQ ID Nos.117-124 for nucleotide sequences encoding SEQ ID Nos. 173-184).

FIG. 15. Receptor binding specificity of A/New York/108/2016 (H7N2) influenza virus isolated from a human who experienced influenza-like illness after exposure to sick domestic cats at an animal shelter in New York, N.Y., USA, 2016. Figure indicates glycan microarray analysis. Colored bars represent glycans that contain α-2,3 sialic acid (SA) (blue), α-2,6 SA (red), α-2,3/α-2,6 mixed SA (purple), N-glycolyl SA (green), α-2,8 SA (brown), β-2,6 and 9-O-acetyl SA (yellow), and non-SA (gray). Error bars reflect SE in the signal for 6 independent replicates on the array. RFI, relative fluorescence intensity.

FIG. 16. Receptor binding specificity of A/New York/108/2016 (H7N2) influenza virus isolated from a human who experienced influenza-like illness after exposure to sick domestic cats at an animal shelter in New York, N.Y., USA, 2016. Figure shows A/New York/108/2016 hemagglutinin (HA) monomer structure. HA1 is shown in gray, HA2 in light purple, amino acid changes in comparison with reference virus A/turkey/Virginia/452912002 (H7N2) in red. On the cartoon view (left), all amino acid changes in the HA protein are labeled. The location of the receptor binding site (blue circle) includes the 120-loop and the 180-helix. The 220-loop is missing due to deletion of amino acids 212-219 in the mature HA protein (H7 numbering). On the surface model (right), only amino acid substitutions adjacent to the antigenic sites and receptor binding site.

### DETAILED DESCRIPTION

### Definitions

As used herein, the term “isolated” refers to in vitro preparation and/or isolation of a nucleic acid molecule, e.g., vector or plasmid, peptide or polypeptide (protein), or virus of the invention, so that it is not associated with in vivo substances, or is substantially purified from in vitro substances. An isolated virus preparation is generally obtained by in vitro culture and propagation, and/or via passage in eggs, and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

As used herein, a “heterologous” influenza virus gene or viral segment is from an influenza virus source that is different than a majority of the other influenza viral genes or viral segments in a recombinant, e.g., reassortant, influenza virus.

The terms “isolated polypeptide”, “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See http://www.ncbi.n1m.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Influenza Virus Type A Structure and Propagation

Influenza A viruses possess a genome of eight single-stranded negative-sense viral RNAs (vRNAs) that encode at least ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cRNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Influenza viruses of three HA subtypes (H1, H2 and H3) have acquired the ability to be transmitted efficiently among humans. In addition, influenza viruses of the H5, H6, H7, H9 and H10 subtypes are also considered to represent pandemic threats since they have crossed the species barrier and infected humans. Both wild and domestic birds are suitable influenza virus hosts that mostly experience asymptomatic infections, despite high virus replication. Viruses of subtypes H5 and H7 are a notable exception, since they can evolve in poultry to become highly pathogenic avian influenza (HPA1) viruses, causing severe disease and mortality. There are nine known subtypes of H7 viruses (H7N1, H7N2, H7N3, H7N4, H7N5, H7N6, H7N7, H7N8, and H7N9). Most H7 viruses identified worldwide in wild birds and poultry are LPAI viruses. In humans, LPAI (H7N2, H7N3, and H7N7) virus infections have caused mild to moderate illness.

Any cell, e.g., any avian or mammalian cell, such as a human, canine, bovine, equine, feline, swine, ovine, mink, e.g., MvLu1 cells, or non-human primate cell, including mutant cells, which supports efficient replication of influenza virus can be employed to isolate and/or propagate influenza viruses. Isolated viruses can be used to prepare a reassortant virus, e.g., an attenuated virus. In one embodiment, host cells for vaccine production are those found in avian eggs. In another embodiment, host cells for vaccine production are continuous mammalian or avian cell lines or cell strains. It is preferred to establish a complete characterization of the cells to be used, so that appropriate tests for purity of the final product can be included. Data that can be used for the characterization of a cell includes (a) information on its origin, derivation, and passage history; (b) information on its growth and morphological characteristics; (c) results of tests of adventitious agents; (d) distinguishing features, such as biochemical, immunological, and cytogenetic patterns which allow the cells to be clearly recognized among other cell lines; and (e) results of tests for tumorigenicity. The passage level, or population doubling, of the host cell used may be as low as possible.

The virus produced by the host cell may be highly purified prior to vaccine or gene therapy formulation. Generally, the purification procedures result in the extensive removal of cellular DNA, other cellular components, and adventitious agents. Procedures that extensively degrade or denature DNA can also be used.

### Exemplary Conditions for Multiplex RT-PCR

Influenza viruses may be isolated in Madin-Darby Canine Kidney (MDCK) cells. RNA extraction may be done using 350 μL of sample with the RNeasy Mini kit (Qiagen, Hilden, Germany) according to the manufacturer's instructions. Influenza virus cDNA may be synthesized using random primer (Promega Corps., USA) and avian myeloblastic reverse transcriptase (AMV RT), (Promega Corp. USA). For a 12.5 μL reaction volume, 5.0 μL of RNA and 500 ng of random primer may be employed along with 200 μM of each deoxynucleaotise triphosphates (dNTPs) (Promega, Corps., USA) and 10 units of AMV RT. The reaction mix may be incubated at 37° C. for 90 minutes, followed by 65° C. for 10 minutes to inactivate the enzyme.

Primers used for detection and typing of influenza virus may target amplification of matrix and/or non-structural gene (NS) for influenza A and influenza B, respectively, and sub-typing of s influenza A may use specific primers from hemagglutinin genes (HA) and neuraminidase genes (NA).

Typing PCR for influenza A and B may be carried out as a monoplex assay with 5 μL of cDNA in a total volume of 25 μL containing 50 picomoles each of forward and reverse primers of influenza A or influenza B, 200 μM each of four dNTPs, and 1.5 units of the Taq polymerase (Bangalore Genei, India). DNA amplification may be performed using an initial denaturation for 3 minutes at 94° C., followed by 35 cycles of denaturation for 1 minute at 94° C., annealing for 1 minute at 52° C. and extension for 1 minute at 72° C., with final extension for 10 minutes at 72° C. in a thermocycler (Gene AMP PCR system 9700, Applied Biosystems, USA). Amplicons may be visualized under a digital gel documentation system (Bio-Rad, UK). PCR mix for multiplex RT-PCR for pandemic may contain 20 picomoles each of primers from matrix gene and 10 picomoles each of primer targeting the HA gene. Rest of the conditions of PCR reaction may be the same as sub-typing multiplex PCR.

A multiplex PCR may be performed with 5 μL of cDNA in 25 μL, reaction volume containing dNTPs, 50 picomoles each of forward and reverse primers for seasonal H1, H3, H7, N1 and N2, 1.5 U of Taq DNA polymerase. DNA amplification may be performed using initial denaturation for 3 minutes at 94° C., followed by 40 cycles of denaturation for 30 seconds at 94° C., annealing for 30 seconds at 50° C. and extension for 30 seconds at 72° C., with final extension for 10 minutes at 72° C. in a thermocycler. Amplicon may be visualized for H1, H3, H7, N1 and N2. In addition, a multiplex RT-PCR for pandemic A may be carried out with primers for matrix gene and HA and/or N4 gene.

### Influenza Vaccines

A vaccine includes an isolated influenza virus as disclosed herein, and optionally one or more other isolated viruses including other isolated influenza viruses, West Nile virus, herpes virus, lentivirus, rabies virus, and/or one or more immunogenic proteins or glycoproteins of one or more isolated influenza viruses or one or more other pathogens, an immunogenic protein from one or more bacteria, non-influenza viruses, yeast or fungi, or isolated nucleic acid encoding one or more viral proteins (e.g., DNA vaccines) including one or more immunogenic proteins of the isolated influenza virus of the disclosure. In one embodiment, the influenza viruses of the disclosure may be vaccine vectors for influenza virus or other pathogens.

A complete virion vaccine may be concentrated by ultrafiltration and then purified by zonal centrifugation or by chromatography. It is inactivated before or after purification using formalin or beta-propiolactone, for instance.

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976); or a nonionic detergent such as that commercialized under the name TRITON X100. The hemagglutinin may also be isolated after treatment of the virions with a protease such as bromelin, then purified by a method such as that described by Grand and Skehel (1972).

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done.

Inactivated Vaccines.

Inactivated influenza virus vaccines are provided by inactivating replicated virus using known methods, such as, but not limited to, formalin or β-propiolactone treatment. Inactivated vaccine types that can be used in the vaccines and methods can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live Attenuated Virus Vaccines.

Live, attenuated influenza virus vaccines can be used for preventing or treating influenza virus infection. Attenuation may be achieved in a single step by transfer of attenuated genes from an attenuated donor virus to a replicated isolate or reassorted virus according to known methods (see, e.g., Murphy, 1993). Since resistance to influenza A virus is mediated primarily by the development of an immune response to the HA and/or NA glycoproteins, the genes coding for these surface antigens must come from the reassorted viruses or clinical isolates. The attenuated genes are derived from the attenuated parent. In this approach, genes that confer attenuation preferably do not code for the HA and NA glycoproteins.

Viruses (donor influenza viruses) are available that are capable of reproducibly attenuating influenza viruses, e.g., a cold adapted (ca) donor virus can be used for attenuated vaccine production. Live, attenuated reassortant virus vaccines can be generated by mating the ca donor virus with a virulent replicated virus. Reassortant progeny are then selected at 25° C., (restrictive for replication of virulent virus), in the presence of an appropriate antiserum, which inhibits replication of the viruses bearing the surface antigens of the attenuated ca donor virus. Useful reassortants are: (a) infectious, (b) attenuated for seronegative non-adult mammals and immunologically primed adult mammals, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible mammals both adults and non-adult.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene (Subbarao et al., 1993). Thus, new donor viruses can also be generated bearing attenuating mutations introduced by site-directed mutagenesis, and such new donor viruses can be used in the production of live attenuated reassortants vaccine candidates in a manner analogous to that described above for the ca donor virus. Similarly, other known and suitable attenuated donor strains can be reassorted with influenza virus to obtain attenuated vaccines suitable for use in the vaccination of mammals (Enami et al., 1990; Muster et al., 1991; Subbarao et al., 1993).

It may be preferred that such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The virus can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g., amantadine or rimantidine); HA and NA activity and inhibition; and nucleic acid screening (such as probe hybridization or PCR) to confirm that donor genes encoding the antigenic determinants (e.g., HA or NA genes) are not present in the attenuated viruses. See, e.g., Robertson et al., 1988; Kilbourne, 1969; Aymard-Henry et al., 1985; Robertson et al., 1992.

Influenza DNA vaccines may be produced as plasmids grown in genetically modified bacteria, normally Escherichia coli. Such plasmids contain, alongside the gene of interest, a bacterial origin of replication and a selective gene, normally encoding antibiotic resistance, to maintain the persistence of the plasmid in the bacterium. DNA vaccines have proven to be safe in a number of animal models and early phase clinical trials.

The use of minimal DNA constructs, for example, minicircles, small circular fragments of DNA derived from a larger plasmid or minimalistic immunologically defined expression (MIDGE) vectors, that only encode an antigne expression cassette (promoter, antigen and polyA region), may eliminate extraneous elements. These vectors have been shown to be immunogenic, inducing both a cellular and humoral response. Synthesizing the DNA vaccine entirely in vitro, with the absence of a bacterial step, would ensure uniformity between batches and increase their readiness for good manufacturing practice (GMP) production.

DNA constructs expressing influenza hemagluttinin (HA) have been tested for their ability to protect mice from challenge with a homologous influenza strain. The DNA induces an immune response to antigen and, when used as a vaccine, can protect from viral infection. Thus, DNA having the NA and/or HA sequences disclosed herein may be employed as vaccines.

VLPs, which resemble infectious virus particles in structure and morphology and have multiple antigenic epitopes, have been developed as non-egg-based, cell culture-derived vaccine candidates against influenza infection. Influenza VLP vaccines containing influenza HA and/or NA antigens may be produced easily in insect or mammalian cells via the simultaneous expression of HA and/or NA along with a viral core protein such as influenza M1. The highly organized form of antigens on VLP scan induce strong B-cell responses. The protective mechanisms of influenza. VLP vaccines, that is, induction of neutralizing antibodies and HA inhibition, are similar to those of commerical influenza vaccines. In addition, VLPs can stimulate antigen presentation cells and induce CD4 T-cell proliferation and cytotoxic T-cell immune responses and thus induce both B- and T-cell responses.

All of the influenza VLPs for veterinary use have been produced using baculovirus/insect cell technology and emulsified with an oil adjuvant. Lee et al. (2013, 2011) developed CIV H3 VLP and LP AI H9 vaccines. CIV H3 VLP vaccines contain HA and M1 but not NA. A single dose of vaccination with that vaccine induced high antibody titers and lessened viral shedding.

Thus, VLPs having the NA and/or HA sequences described herein, may be employed as vaccines.

### Pharmaceutical Compositions

Pharmaceutical compositions, suitable for inoculation, e.g., nasal, parenteral or oral administration, comprise one or more influenza virus isolates, e.g., one or more attenuated or inactivated influenza viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. See, e.g., Berkow et al., 1987; Avery's Drug Treatment, 1987; Osol, 1980. The composition is generally presented in the form of individual doses (unit doses).

Conventional vaccines generally contain about 0.1 to 200 μg, e.g., 30 to 100 μg, but may include about 1 to 30 μg, 1 to 50 μg, 10 to 50 μg, 10 to 30 μg, 30 to 100 μg, or 50 to 100 μg, of HA from each of the strains entering into their composition. The vaccine forming the main constituent of the vaccine composition may comprise a single influenza virus, or a combination of influenza viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable foul's for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow et al., 1992; Avery's, 1987; and Osol, 1980.

When a composition is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized. Examples of materials suitable for use in vaccine compositions are provided in Osol (1980).

Heterogeneity in a vaccine may be provided by mixing replicated influenza viruses for at least two influenza virus strains, such as 2-20 strains or any range or value therein. Influenza A virus strains having a modern antigenic composition are preferred. Vaccines can be provided for variations in a single strain of an influenza virus, using techniques known in the art.

A pharmaceutical composition may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions which are vaccines are provided before any symptom or clinical sign of a pathogen infection becomes manifest. The prophylactic administration of the composition serves to prevent or attenuate any subsequent infection. When provided prophylactically, the gene therapy compositions are provided before any symptom or clinical sign of a disease becomes manifest. The prophylactic administration of the composition serves to prevent or attenuate one or more symptoms or clinical signs associated with the disease.

When provided therapeutically, an attenuated or inactivated viral vaccine is provided upon the detection of a symptom or clinical sign of actual infection. The therapeutic administration of the compound(s) serves to attenuate any actual infection. See, e.g., Berkow et al., 1992; and Avery, 1987. When provided therapeutically, a gene therapy composition is provided upon the detection of a symptom or clinical sign of the disease. The therapeutic administration of the compound(s) serves to attenuate a symptom or clinical sign of that disease.

Thus, an attenuated or inactivated vaccine composition may be provided either before the onset of infection (so as to prevent or attenuate an anticipated infection) or after the initiation of an actual infection. Similarly, for gene therapy, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition is physiologically significant if its presence results in a detectable change in the physiology of a recipient patient, e.g., enhances at least one primary or secondary humoral or cellular immune response against at least one strain of an infectious influenza virus.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition may confer resistance to one or more pathogens, e.g., one or more influenza virus strains, by either passive immunization or active immunization. In active immunization, an inactivated or attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one influenza virus strain. A gene therapy composition may yield prophylactic or therapeutic levels of the desired gene product by active immunization.

In one embodiment, the vaccine is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The disclosure thus includes methods for preventing or attenuating a disorder or disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease. As used herein, a gene therapy composition is said to prevent or attenuate a disease if its administration results either in the total or partial attenuation (i.e., suppression) of a clinical sign or condition of the disease, or in the total or partial immunity of the individual to the disease.

At least one influenza virus isolate, including one which is inactivated or attenuated, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present disclosure, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the doses and represent dose ranges.

The dosage of a live, attenuated or killed virus vaccine for an animal such as a mammalian adult organism can be from about 10²-10¹⁵, e.g., 10³-10¹², 10⁵-10⁸, 10⁷-10¹², 10⁹-10¹², 10¹⁰-10¹², or 10³-10⁹ plaque forming units (PFU)/kg, or any range or value therein. In one embodiment, the dosage of a live, attenuated or killed virus vaccine for an animal such as a mammalian adult organism can be from about 10²-10¹⁵, e.g., 10³-10¹², 10⁵-10⁸, 10⁷-10¹², 10⁹-10¹², 10¹⁰-10¹², or 10³-10⁹ plaque forming units (PFU), or any range or value therein. The dose of vaccine, such as an inactivated vaccine can range from about 0.1 to 1000, e.g., 30 to 200 μg, such as 5 to 20 μg, 30 to 50 μg, 50 to 100 μg or 150 to 200 μg, of HA protein, e.g., per at least a 15-40 pound mammal. The dose of vaccine, such as an inactivated vaccine can range from about 0.1 to 1000, e.g., 30 to 200 μg, such as 5 to 20 μg, 30 to 50 μg, 50 to 100 μg or 150 to 200 μg, of HA protein, e.g., per at least a 40 to 300 (or more) pound mammal. However, the dosage should be a safe and effective amount as determined by conventional methods, using existing vaccines as a starting point.

The dosage of immunoreactive HA in each dose of replicated virus vaccine can be standardized to contain a suitable amount, e.g., 30 to 200 μg such as 30 to 100 μg or such as 30 to 50 μg, or any range or value therein, or the amount recommended by government agencies or recognized professional organizations. The quantity of NA can also be standardized, however, this glycoprotein may be labile during purification and storage.

### Exemplary Compositions for Influenza Vaccines

Influenza vaccines may include representative strains of H7N2, as disclosed herein, either as, for example, inactivated whole virus or their subunits, or live attenuated virus. They provide protection against influenza by inducing antibody to the surface glycoproteins, in particular to HA, which is essential for viral attachment and entry into cells, and/or potentially important cell-mediated immune responses to other viral proteins. Vaccination is helpful in preventing influenza but the protection is relatively short-lived (e.g., 3-4 months using conventional inactivated virus vaccines), so the frequency of vaccination varies. One procedure for vaccination is a single dose followed by a second dose. Alternatively, a vaccine is administered, for example, for domestic cats in one 0.1 to 2.0 mL dose, e.g., via intramuscular (IM) injection, or 0.1 to 0.5 mL intranasal administration, and optionally, a second 0.1 to 0.5 mL dose alter, e.g., 2 to 4 weeks later, e.g., via IM injection, and optionally a third 0.1 to 0.5 mL dose, e.g., IM or intranasal (IN) administration. Each dose of vaccine may contain approximately 0.1-125 billion virus particles.

Influenza vaccines may be combined with other vaccinations, e.g., other feline pathogens.

Levels of antibody (measured by the SRH assay) required for protection may be identified through vaccination and challenge studies and from field data. Because the vaccine-induced antibody response to HA may be short-lived, adjuvants such as aluminum hydroxide or carbomer may be included to enhance the amplitude and duration of the immune response to whole virus vaccines. Subunit influenza vaccines containing immune stimulating complexes (ISCOMs) are also immunogenic.

Historically, antigenic content in inactivated vaccines has been expressed in terms of chick cell agglutinating (CCA) units of HA and potency in terms of HI antibody responses. The single radial diffusion (SRD) assay is an improved in vitro potency test that measures the concentration of immunologically active HA (expressed in terms of micrograms of HA) and can be used for in-process testing before the addition of adjuvant.

### EXEMPLARY EMBODIMENTS

In one embodiment, a vaccine having an effective amount of isolated H7 influenza virus comprising a viral HA segment with sequences for a HA-1 having greater than 92% amino acid sequence identity to HA-1 encoded by a nucleotide sequence having one of SEQ ID Nos. 45-52, 54-64, 85, 93, 101, 109, or 117 or encoding over 90%, 92%, 95%, 96%, 97%, 98% or 99% amino acid sequence identity to HA comprising one of SEQ ID Nos. 125, 137, 149, 161, or 173. In one embodiment, the influenza virus comprises a viral HA segment with sequences for a HA-1 having greater than 92%, 95%, or 99% amino acid sequence identity to HA-1 encoded by a nucleotide sequence having one of SEQ ID Nos. 44-52, 54-64, 85, 93, 101, 109, or 117 or encoding over 90%, 92%, 95%, 96%, 97%, 98% or 99% amino acid sequence identity to HA comprising one of SEQ ID Nos. 78, 125, 137, 149, 161, or 173. In one embodiment, the H7 influenza virus comprises a viral HA segment with sequences for a HA-1 having greater than 95% amino acid sequence identity to HA-1 encoded by a nucleotide sequence having one of SEQ ID Nos. 45-52, 54-64, 85, 93, 101, 109, or 117. In one embodiment, the H7 influenza virus comprises a viral HA segment with sequences for a HA-1 having greater than 99% amino acid sequence identity to HA-1 encoded by a nucleotide sequence having one of SEQ ID Nos. 45-52, 54-64, 85, 93, 101, 109, or 117. In one embodiment, the H7 influenza virus has a residue in the HA-1 at position 84 is not T, at position 104 is not G or R, at position 109 is not G, D or 5, at position 125 is not A or T, at position 180 is not S or T, at position 183 is not T, at position 188 is not 5, at position 203 is not S, at position 292 is not T, or any combination thereof. In one embodiment, the H7 influenza virus has a residue in HA-1 at position 84 that is N or Q, at position 104 that is K, R or H, at position 109 that is N or E, at position 125 that is S, at position 180 that is N or Q, at position 183 is I (isoleucine), L or G, at position 188 is N or Q, at position 203 that is P (proline), at position 292 that is I, L or G, or any combination thereof. In one embodiment, the H7 influenza virus has a residue at position 24, 36, 86, 93, 138, 151, 158, 177, 258, 269, 292, or any combination that is serine, alanine, valine, isoleucine, glycine or threonine, and/or a residues at position 290 that is proline, serine, alanine, value, isoleucine, glycine or threonine. In one embodiment, the H7 influenza virus has a NA having at least 95%, 97%, or 98% amino acid sequence identity to a polypeptide encoded by a nucleotide sequence having one of SEQ ID Nos. 1-18, 127, 135, 143, 151, or 159. In one embodiment, the residue at position 127 is serine, alanine, leucine, isoleucine, threonine, or glycine. In one embodiment, the residue at position 156 is alanine, leucine, isoleucine, serine, or glycine. In one embodiment, the vaccine further comprises a different isolated influenza virus or antigen of a non-influenza microbial pathogen. In one embodiment, the isolated influenza virus is an attenuated virus. In one embodiment, the isolated influenza virus is a reassortant virus. In one embodiment, the influenza virus has been altered by chemical, physical or molecular means. In one embodiment, the virus is inactivated. In one embodiment, the vaccine further comprises an adjuvant. In one embodiment, the vaccine further comprises a pharmaceutically acceptable carrier. In one embodiment, the carrier is suitable for intranasal orintramuscular administration. In one embodiment, the vaccine is in freeze-dried form.

In one embodiment, a pharmaceutical composition is provided comprising an amount of isolated HA with sequences for a HA-1 having greater than 92% amino acid sequence identity to HA-1 encoded by a nucleotide sequence having one of SEQ ID Nos. 45-52, 54-64, 85, 93, 101, 109, or 117 or encoding over 99% amino acid sequence identity to HA comprising one of SEQ ID Nos. 125, 137, 149, 161, or 173, or isolated nucleic acid encoding the HA, effective to induce a protective immune response. In one embodiment, a pharmaceutical composition is provided comprising an amount of isolated HA with sequences for a HA-1 having greater than 92% amino acid sequence identity to HA-1 encoded by a nucleotide sequence having one of SEQ ID Nos. 44-52, 54-64, 85, 93, 101, 109, or 117 or encoding over 99% amino acid sequence identity to HA comprising one of SEQ ID Nos. 78, 125, 137, 149, 161, or 173, or isolated nucleic acid encoding the HA, effective to induce a protective immune response.

In one embodiment, a method to prepare influenza virus is provided, comprising: contacting an avian or mammalian cell with an isolated H7 influenza virus comprising a viral HA segment with sequences for a HA-1 having greater than 92% amino acid sequence identity to a polypeptide encoded by a nucleotide sequence having one of SEQ ID Nos. 45-52, 54-64, 85, 93, 101, 109, or 117 or encoding over 99% amino acid sequence identity to HA comprising one of SEQ ID Nos. 125, 137, 149, 161 or 173. In one embodiment, the method includes isolating the virus. In one embodiment, the method employs an influenza virus having a HA with sequences for a HA-1 having greater than 92% amino acid sequence identity to HA-1 encoded by a nucleotide sequence having one of SEQ ID Nos. 45-52, 54-64, 85, 93, 101, 109, or 117 or encoding over 99% amino acid sequence identity to HA comprising one of SEQ ID Nos. 125, 137, 149, 161, or 173, or isolated nucleic acid encoding the HA, effective to induce a protective immune response. In one embodiment, the cell is in an embryonated egg, or is a feline cell or a MDCK cell.

In one embodiment, a method of preparing a vaccine is provided. In one embodiment, an amount of the virus is combined with an adjuvant or is treated with an agent that inactivates the virus, thereby providing a composition; and is then provided an individual dose of the composition comprising an amount effective to immunize a mammal.

Also provided is a method to immunize a mammal against influenza. In one embodiment, the method includes administering to the mammal a composition comprising an effective amount of an isolated H7 influenza virus comprising a viral HA segment with sequences for a HA-1 having greater than 92% amino acid sequence identity to a polypeptide encoded by a nucleotide sequence having one of SEQ ID Nos. 44-52, 54-64, 85, 93, 101, 109, or 117 or encoding over 99% amino acid sequence identity to HA comprising one of SEQ ID Nos. 78, 125, 137, 149, 161 or 173. In one embodiment, the mammal is a human or a feline. In one embodiment, the administration is intranasal, intramuscular, subcutaneous, ocular or oral.

The invention will be further described by the following non-limiting examples.

### Example 1

The present disclosure relates to a new strain of feline H7N2 influenza isolated from an outbreak of influenza among cats at New York City's Manhattan Animal Care Center (ACC-Manhattan). The virus appears to be related to a low pathogenic avian influenza (LPAI) H7N2, a rare subtype that has not been found previously in domestic felines, though this subtype has previously been known to infect avians and humans. Several cases of H7N2 were found in commercial poultry in the United States between 2000 and 2006, and it may be able to spread to other animals. There have been only two cases of H7N2 found in humans, and both cases ended with full recovery.

Cats that contracted the H7N2 strain in the shelter displayed upper respiratory symptoms such as runny nose, congestion, persistent cough and lip smacking, but the illness was not been severe. One cat was euthanized after developing pneumonia. No other species of animals from the shelter, including dogs, tested positive for the virus.

Despite the expected nucleotide differences in the genome between /feline/New York/16-040082-1/2016 (feline/NY/2016) and previously detected LPAI H7N2 viruses from North America, the analysis shows the virus is related to other H7N2 viruses. Although the internal genes are somewhat distant from sequence available in databases, they are all LPAI avian-derived genes with no major mammalian adaptive changes identified. The long branch lengths likely represent a scarcity of sequence data available for contemporary H7N2 viruses.

The deletion of the 220 loop of the receptor binding site (deletion of amino acids 212-219, which was shown to enhance 2,6 binding in some North American lineage H7 viruses) stands out as the major feature likely to allow mammalian infection of this lineage of H7N2 viruses. However, this does not seem to be an all or none correlation.

There are two additional changes (positions 125 and 183) between feline/NY/2016 and other LPAI H7N2 poultry viruses that have been shown to influence in vitro receptor binding specificity for other viruses. A gain of glycosylation at position 125 (which the feline virus has) was also correlated with increased replication efficiency and wider tissue distribution of Netherlands/219 (the H7N7 fatal case).

There are also several differences in predicted antigenic sites (n=6) between feline/NY/2016 vs. NY 1107 and tk/VA.

The sequence is unusual for several reasons. On the tree it looks most closely related to viruses from around 2000, but it has a long branch length. It does have the characteristic LBM marker of a 24 nt deletion right at the receptor binding site. This deletion has only been seen in the H7 LBM lineage. All the other genes are also H7 LBM lineage and the NA has the stalk deletion. The NA stalk deletion is a general marker of a poultry isolate.

### Example 2

Influenza A viruses infect many mammalian and avian hosts. Determination of the subtype, pathotype, and virus lineage is a critical step when confronted with the emergence of an influenza strain in new species. In December 2016, influenza A (H7N2) was first detected among cats in the New York City shelter system with subsequent widespread transmission.

In November of 2016, a severely ill cat showing clinical signs of respiratory disease was euthanized in a New York City animal shelter. A specimen was sent to IDEXX Reference Laboratories for a respiratory PCR panel. An influenza A virus with an N2 subtype was detected and initially reported as presumptive H3N2 canine influenza. However, in consultation with the University of Wisconsin-Madison Shelter Medicine Program, it was recognized that the pattern of transmission among cats, as well as the notable lack of detection in dogs housed in the same facilities, required further investigation. Additional specimens were submitted to the Wisconsin Veterinary Diagnostic Lab and by IDEXX to the California Animal Health and Food Safety Laboratory. Subtype determination by gene-specific PCR and Sanger sequence analysis revealed an H7N2 influenza A virus. Specimens were analyzed simultaneously at the National Veterinary Services Laboratory, confirming North American lineage H7N2 based upon next-generation sequencing direct from the sample and from a recovered virus (Lee et al., 2016).

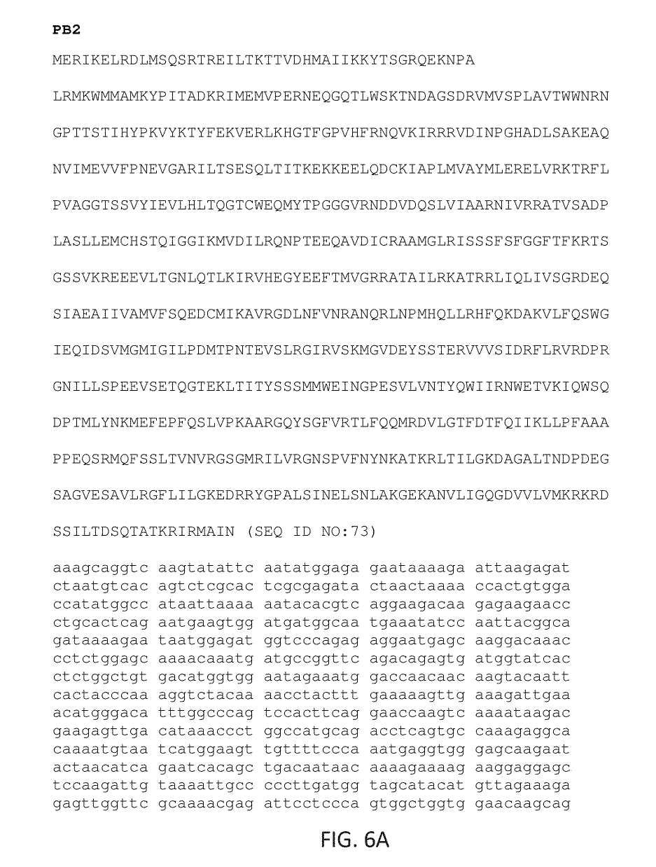

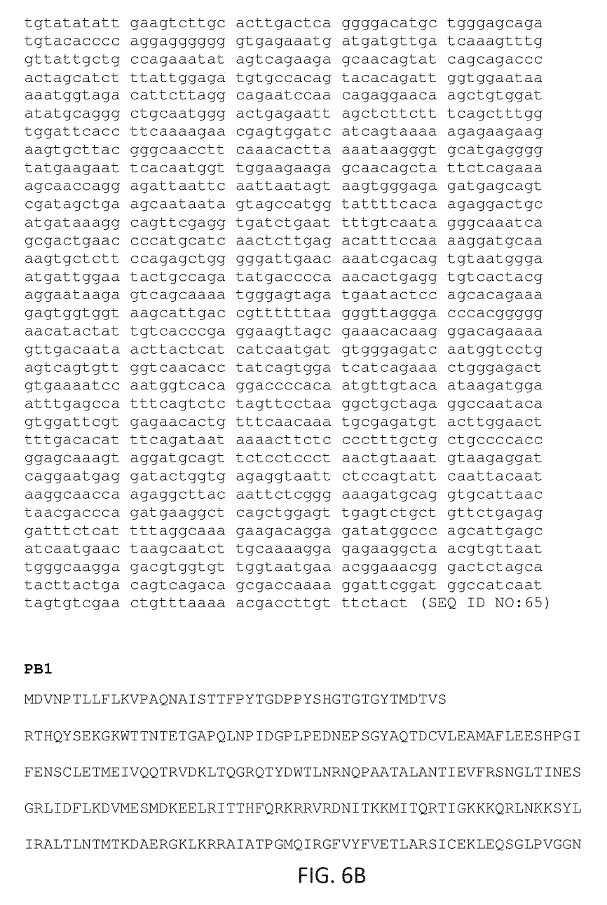

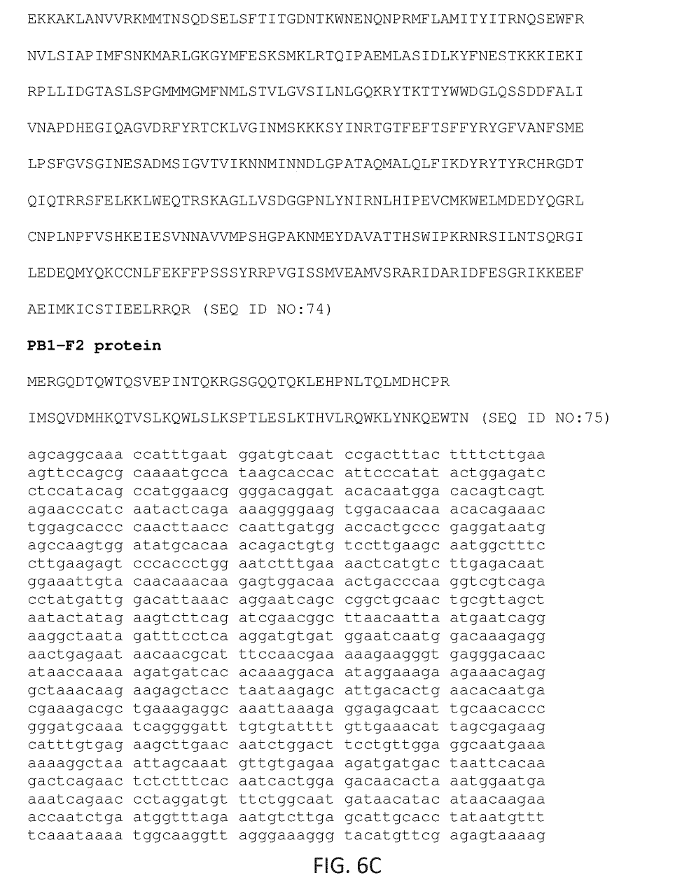

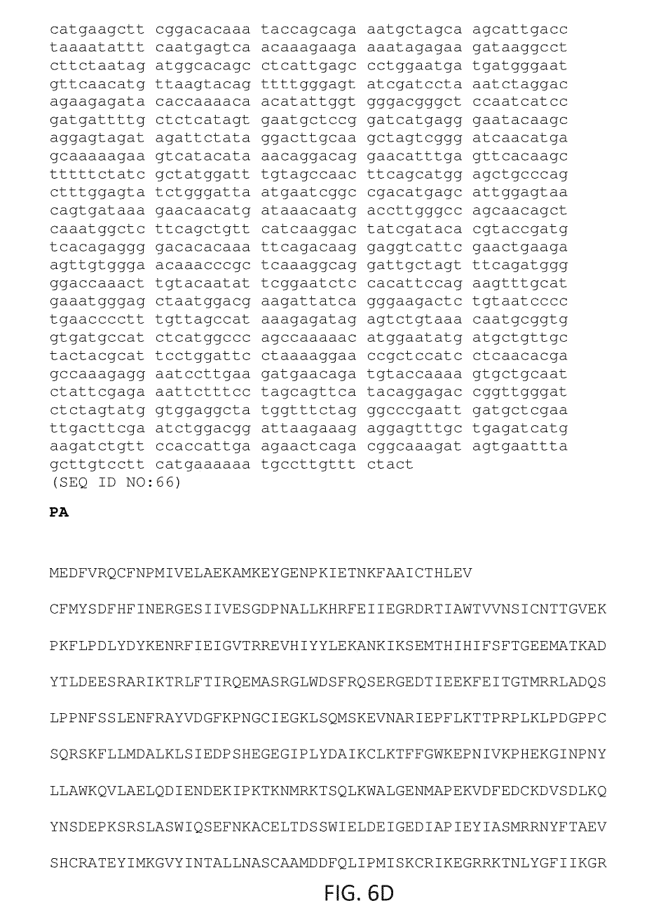

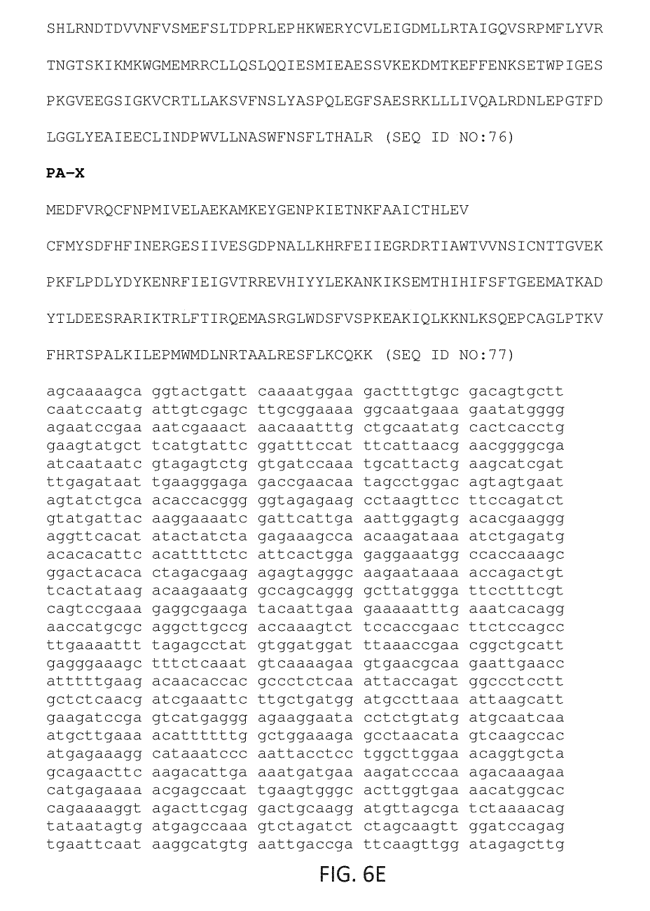

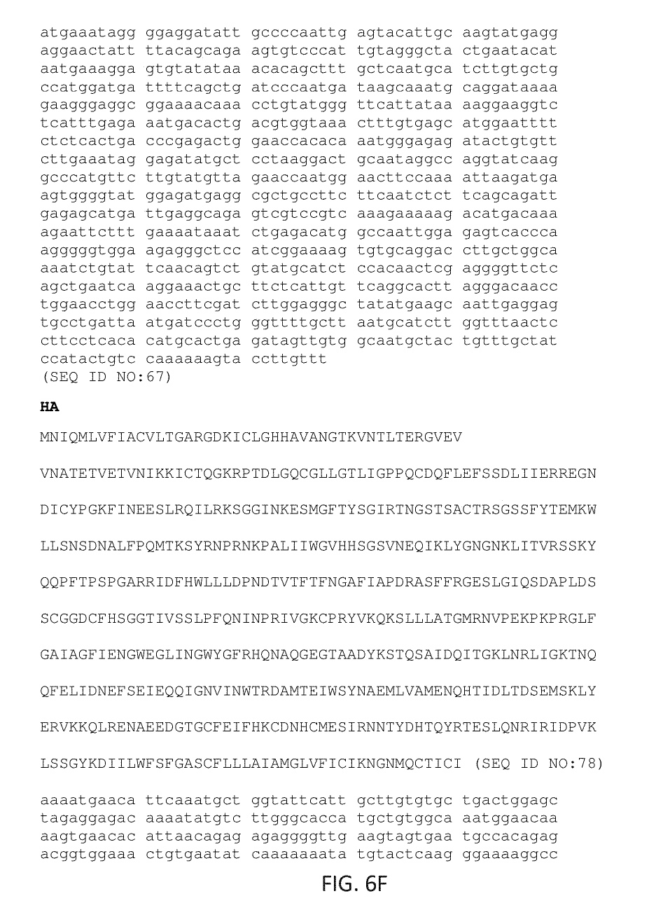

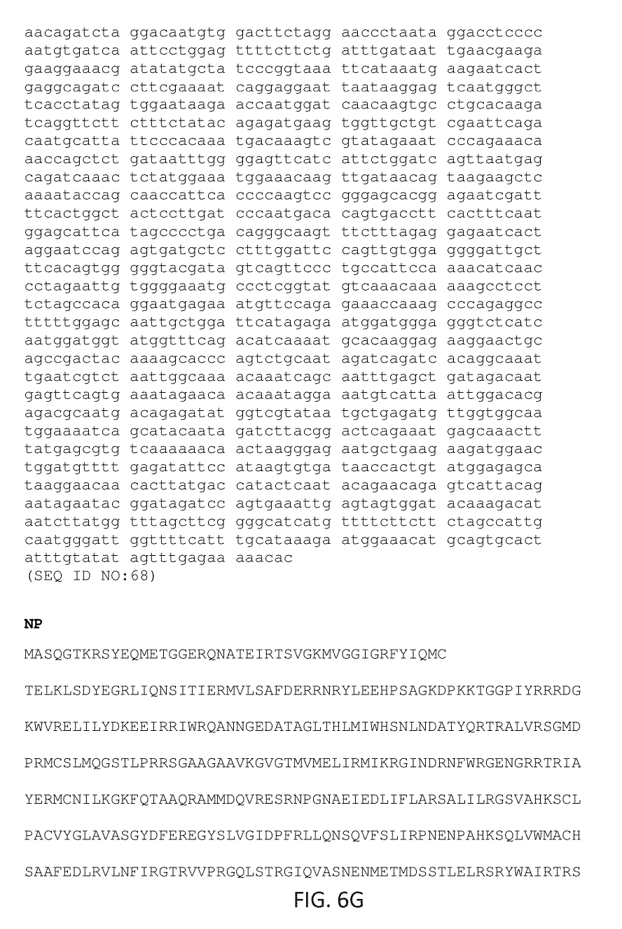

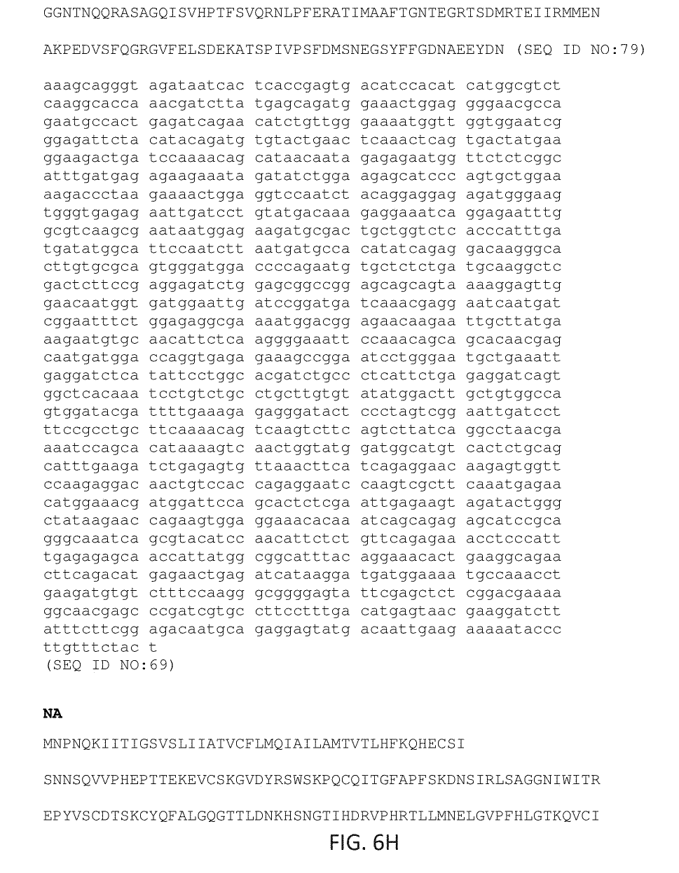

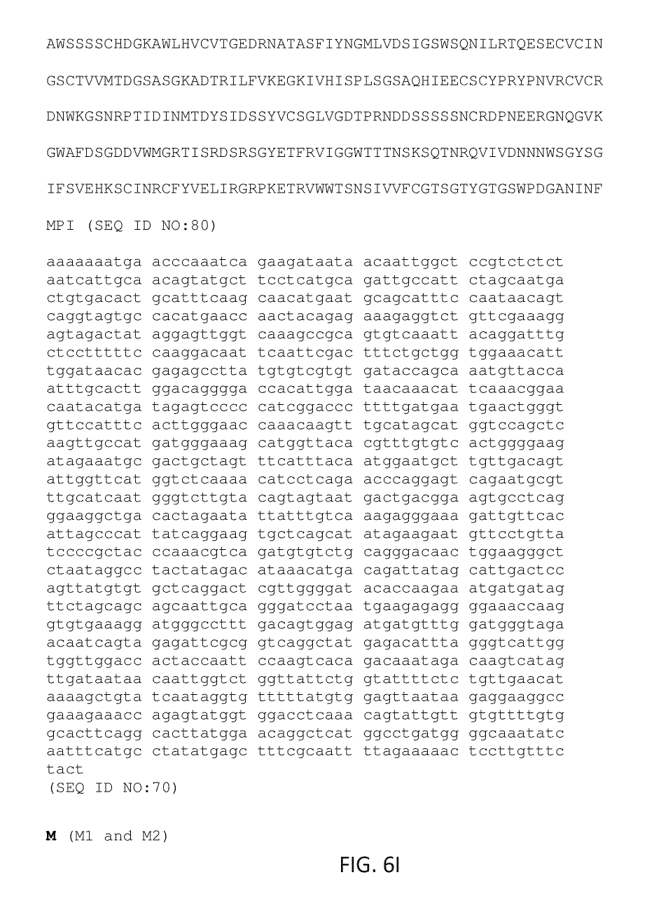

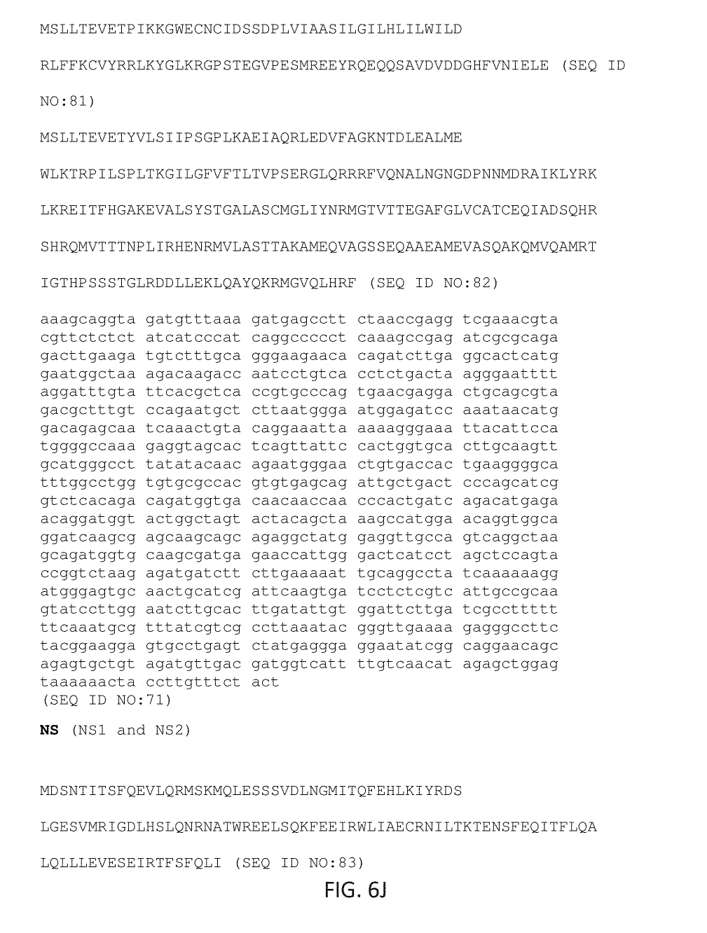

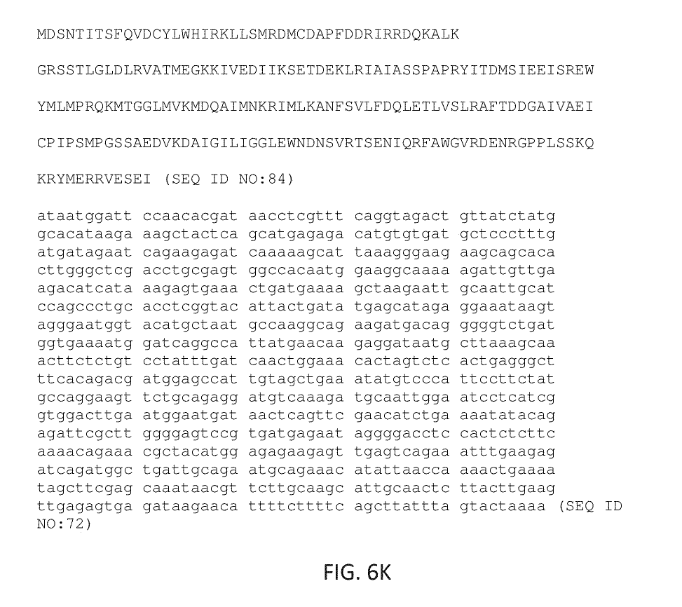

Genome analysis from the first clinical case (A/feline/New York/16-040082-1/2016) indicated that all eight genes (FIG. 6) were highly related to H7N2 live bird market (LBM) lineage low-pathogenic avian influenza (LPAI) viruses that were eradicated from LBM poultry in 2006. Sequence similarity of 96 to 98% at the nucleotide level (95 to 99% at the amino acid level) was closest to LBM strains from the early 2000s. The feline H7N2 viral sequence had several changes associated with increased adaptation for pathogenicity in mammals, including aspartic acid at position 701 of the PB2 protein, serine at position 66 of the PB1-F2 protein, and aspartic acid at position 30 and alanine at position 215 of the matrix 1 protein. However, these same changes were present in all of the historic H7N2 LPAI viruses of the LBM lineage and therefore are unlikely to be the result of current adaptation to cats (Fan et al., 2008; Li et al., 2005; Schmolke et al., 2011).

Detection of avian lineage influenza A strains in cats has been previously documented. Cats have been infected by the highly pathogenic strains H5N1 and H7N7 with limited transmission (Kuiken et al., 2004; van Riel et al., 2010). Other low-pathogenic strains (H6N4 and H1N9) have been experimentally introduced into cats (Driskell et al., 2013), but we are unaware of other avian lineage strains being transmitted with ease, as demonstrated by the spread among New York City shelter cats in late 2016 that infected several hundred cats.

### Example 3

Influenza A viruses are endemic in humans and enzootic in other mammalian species including swine and horses; occasional infections of other mammalian species including whales, seals, sea lions, felidae in zoos, and other species have been reported (Wright et al., 2013). Reports of influenza A virus infections in dogs and cats were rare until 2004, when equine influenza viruses of the H3N8 subtype caused outbreaks in greyhounds in Florida (Crawford et al., 2005). Since then, influenza viruses of the H3N8 and H3N2 subtypes have caused several outbreaks in dogs in the United States and South Korea (Xie et al., 2016; Song et al., 2008; Lee et al., 2012).

Until recently, only 1 major influenza. A virus outbreak had been reported in cats (Fiorentini et al., 2009). This changed in December 2016 with the outbreak of low pathogenic avian influenza A viruses of the H7N2 subtype in animal shelters in New York, During December 2016-February 2017, influenza A viruses of the H7N2 subtype infected about 500 cats in animal shelters in New York, N.Y., USA, indicating virus transmission among cats. Most of the cats experienced a mild illness with coughing, sneezing, and runny nose from which they recovered fully. Severe pneumonia developed in 1 elderly animal with underlying health issues, which was euthanized. A veterinarian who had treated an infected animal also became infected with the feline influenza A(H7N2) virus and experienced a mild, transient illness, including respiratory symptoms, suggesting the potential for these viruses to infect humans.

To understand the pathogenicity and transmissibility of these feline H7N2 viruses in mammals, they were characterized in vitro and in vivo. As described below, feline H7N2 subtype viruses replicated in the respiratory organs of mice, ferrets, and cats without causing severe lesions. Direct contact transmission of feline H7N2 subtype viruses was detected in ferrets and cats; in cats, exposed animals were also infected via respiratory droplet transmission. These results suggest that the feline H7N2 subtype viruses could spread among cats and also infect humans. Outbreaks of the feline H7N2 viruses could, therefore, pose a risk to public health. The ferret findings were in contrast to the findings recently published by Belser et al. (2017).

### Methods

### Cells and Viruses

The origins and growth conditions of all cell lines used in this study are described at https://wwwnc.cdc.gov/EID/article/24/1/17-1240-Techapp1.pdf. The feline H7N2 subtype viruses used in this study were isolated from swabs collected from cats with influenza-like symptoms during the outbreak in an animal shelter in New York in December 2016. A/chicken/New York/22409-4/1999 (H7N2, A/chicken/NY/99) virus was obtained from the Agricultural Research Service, US Department of Agriculture (Spackman et al., 2003). The feline virus was amplified in Madin-Darby canine kidney (MDCK) cells and the A/chicken/NY/99 virus in 10-day-old embryonated chicken eggs.

### Growth Kinetics of Viruses in Cell Culture

Cells were infected with viruses at a 0.005 multiplicity of infection, incubated them for 1 hour at 37° C., washed twice, and cultured with 1×minimal essential medium containing 0.3% bovine serum albumin and trypsin treated with L-1-tosylamide-2-phenylethyl chloromethyl ketone at 33° C. and 37° C. (37° C. and 39° C. for chicken embryo fibroblast cells) for various periods. Virus titers at the indicated time points were determined by use of plaque assays in MDCK cells. The statistical analyses are described in the online Technical Appendix.

### Infection of Animals

To determine the pathogenicity of the viruses in infected mice, three 6-week-old female BALB/c mice (Jackson Laboratory, Bar Harbor, Me., USA) for each virus were anesthetized with isoflurane and inoculated intranasally with 10-fold serially diluted virus in a 50-μL volume. The mice were monitored daily for 14 days and checked for Changes in body weight and morbidity and mortality. Animals were euthanized if they lost more than 25% of their initial bodyweight.

To determine the pathogenicity of the viruses in infected ferrets and cats, 6-month-old female ferrets (Triple F Farms, Sayre, P A, USA; 3 per group; serologically negative by hemagglutination inhibition assay for currently circulating human influenza viruses), and unvaccinated 4- to 5-month-old female specific-pathogen-free cats (Liberty Research, Waverly, N.Y., USA; 3 per group) were inoculated intranasally with 10⁶ PFU of viruses in 0.5 ml of phosphate-buffered saline. The animals were monitored daily for changes in bodyweight, body temperature, and clinical signs for 14 days.

For virus replication in organs and pathology analyses, groups of mice (12 per group), ferrets (6 per group), and cats (6 per group) were used. The animals intranasally with 10⁵ PFU (ice) in 0.05 ml of phosphate-buffered saline or 10⁶ PFU (ferrets and cats) of viruses in 0.5 ml of phosphate-buffered saline. On days 3 and 6 postinfection, we euthanized 6 mice, 3 ferrets, and 3 cats in each group for pathological analysis and virus titration in organs (by use of plaque assays in MDCK cells).

### Virus Transmission Studies in Ferrets and Cats

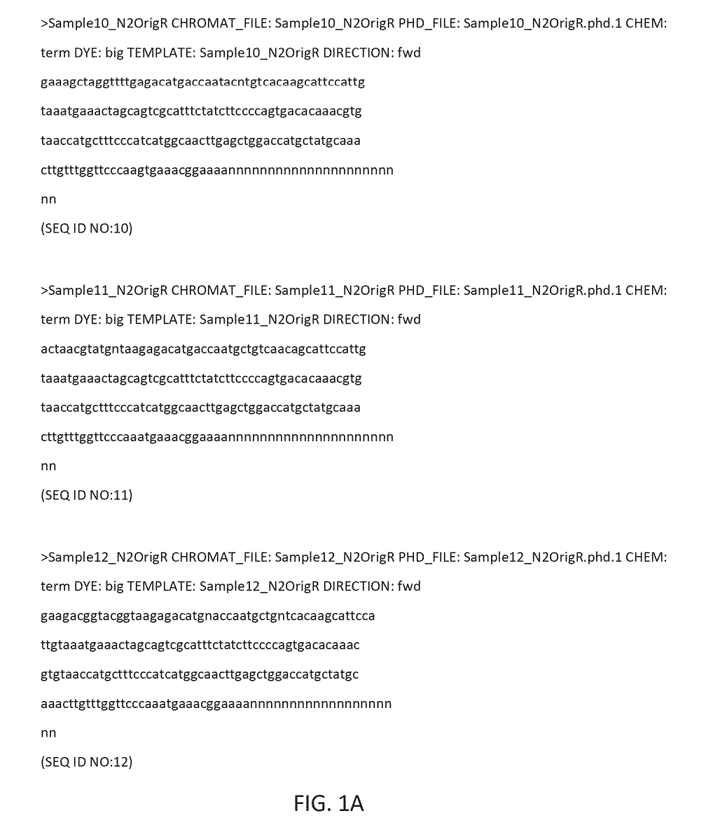

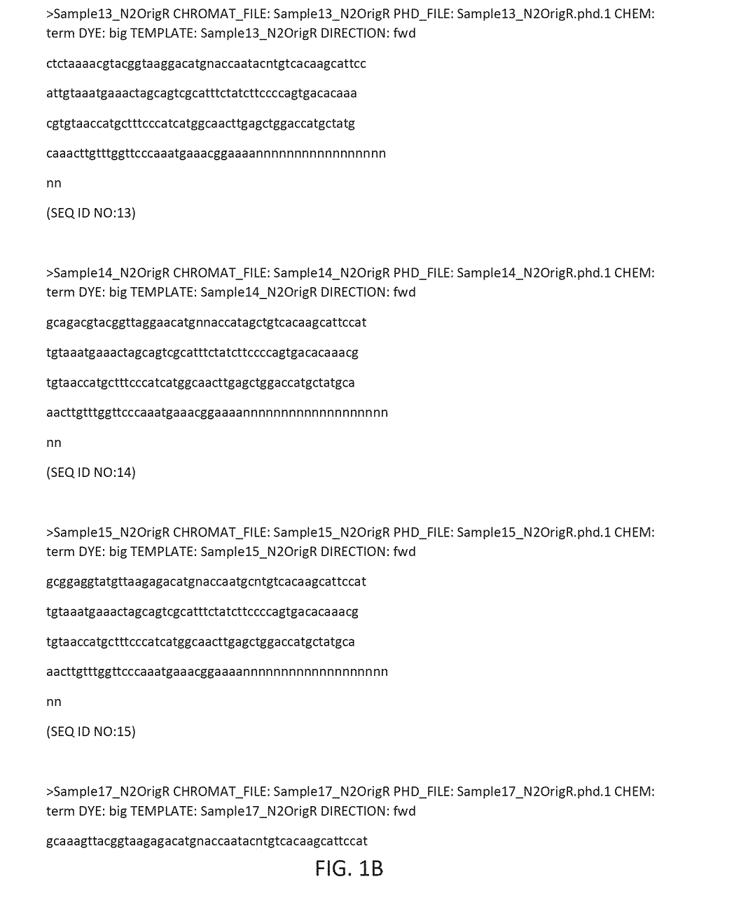

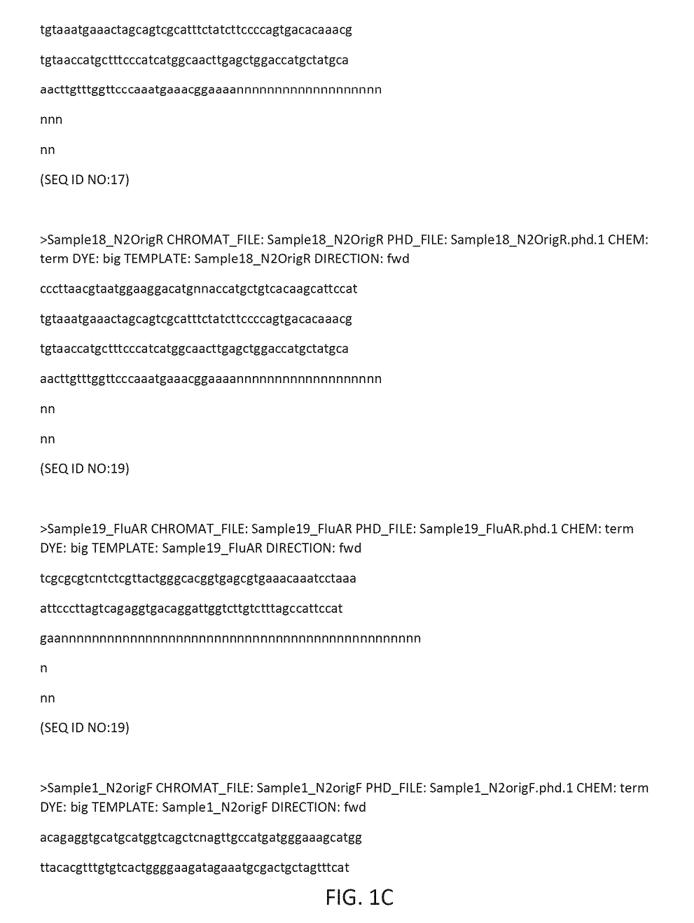

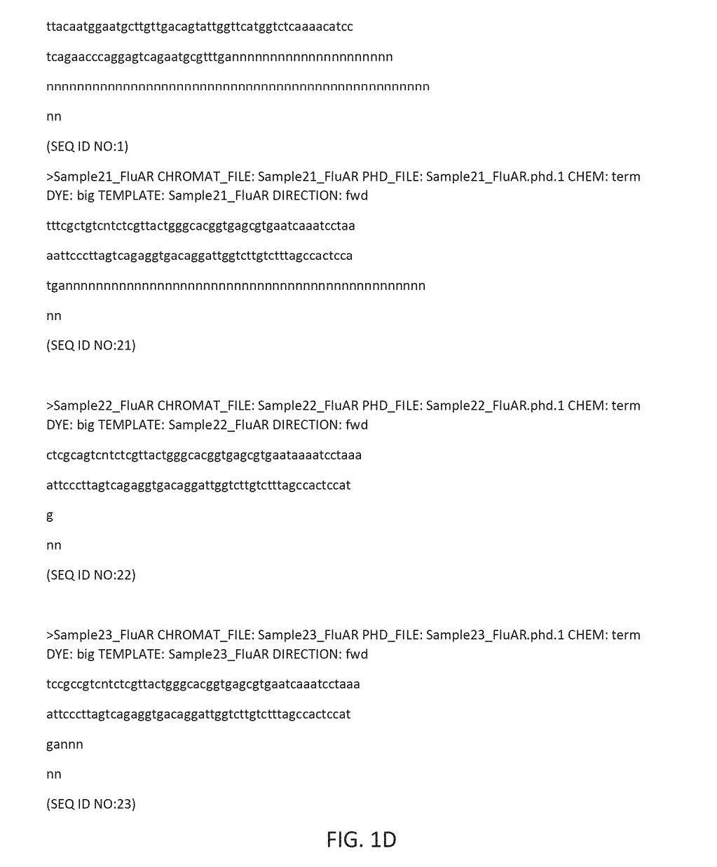

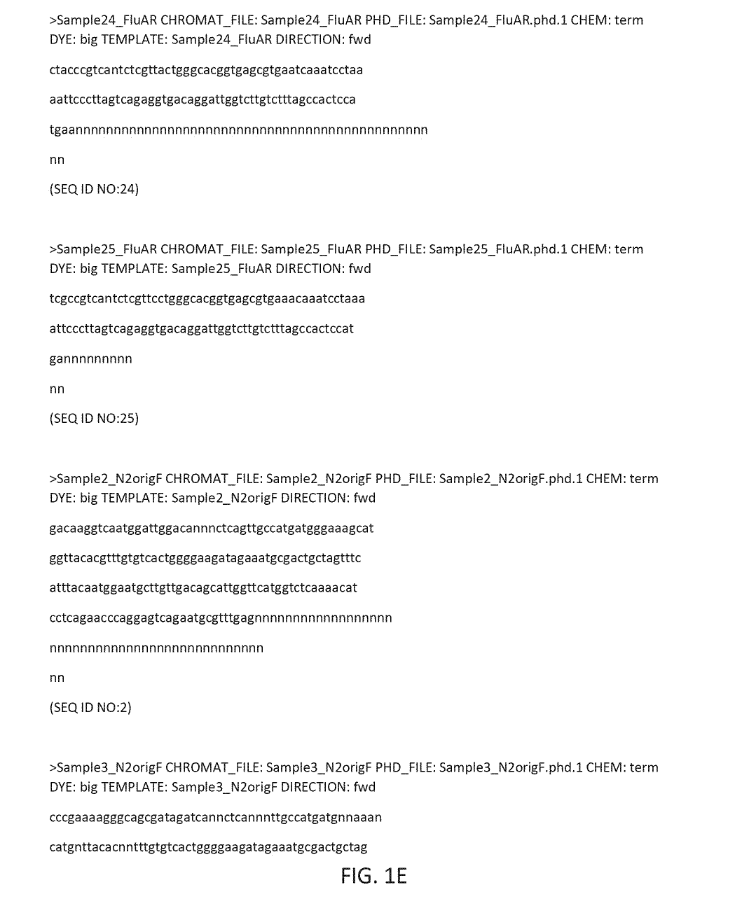

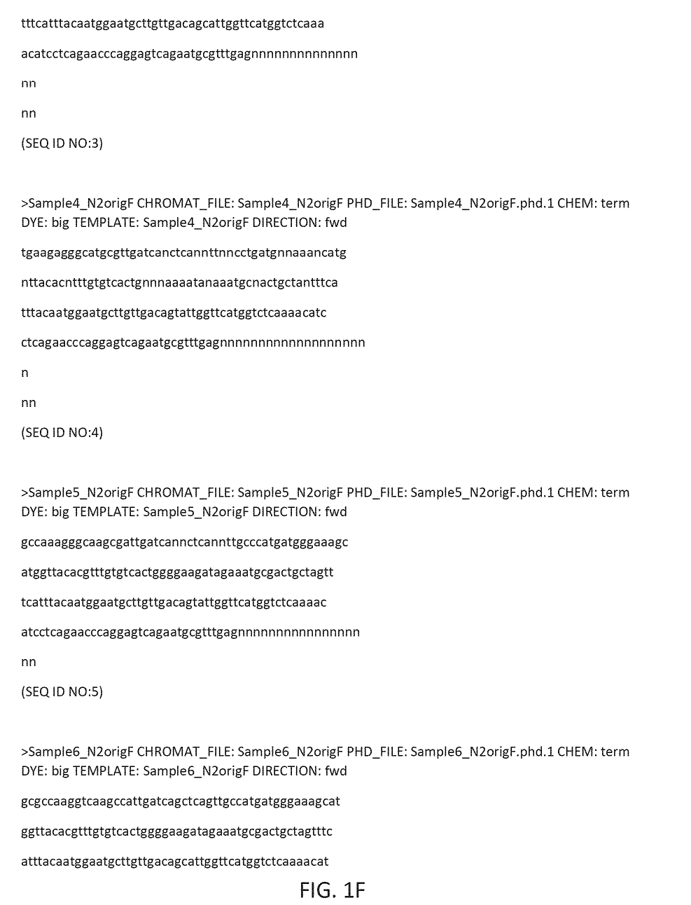

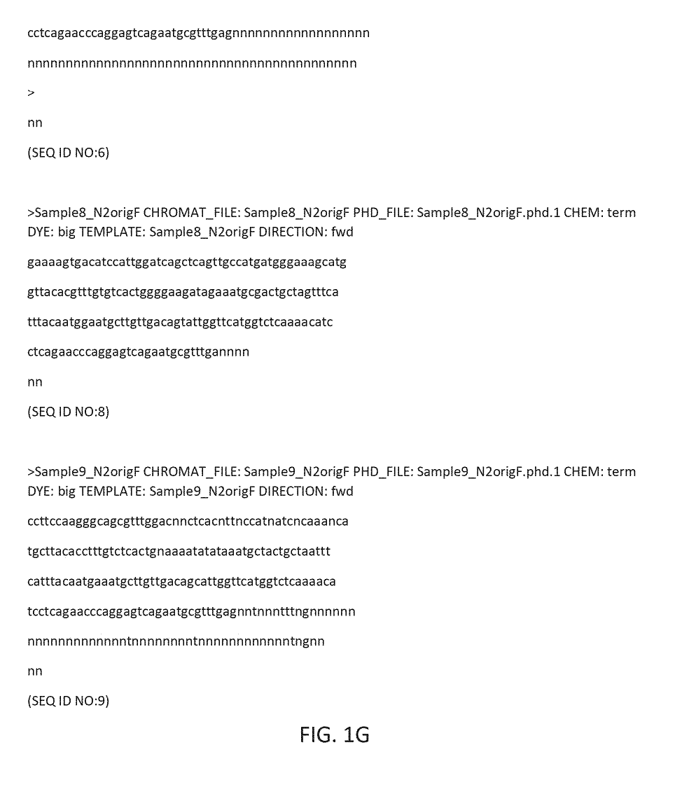

For direct contact transmission experiments, 3 ferrets per group were housed in regular ferret cages and 3 cats per group in large dog transporter cages (online Technical Appendix FIG. 1), and infected them intranasally with 10⁶ PFU (500 μL) of viruses. One day later, 1 virus-naive animal was housed with each infected animal. Nasal washes were collected from the infected ferrets and nasal swabs from the infected cats on day 1 after infection, and from the exposed animals on day 1 after exposure and then every other day (for up to 11 days). Virus titers in the nasal washes and swabs were determined by performing plaque assays in MDCK cells. All animals were monitored daily for disease symptoms and changes in bodyweight and temperature for 14 days.

Airborne transmission experiments were performed by using ferret isolators (Showa. Science, Tokyo, Japan) (Imai et al, 2012; Watanabe et al., 2013; Arafa et al., 2016) or regular cat cages. In these settings, there was no directional airflow from the infected to the exposed animals. 3 animals per group were inoculated intranasally with 10⁶ PFU (500 μL) of viruses. One day after infection, 3 immunologically naive animals (exposed animals) were placed each in a cage adjacent to an infected animal. This setting prevented direct and indirect contact between animals but allowed spread of influenza virus by respiratory droplet. The ferret cages were spaced 5 cm apart and the cat cages 35 cm apart. The animals were monitored and virus titers assessed as described above.

### Results

### Genetic and Phylogenetic Analysis of Feline Influenza (H7N2) Viruses Isolated in Animal Shelters in New York, December 2016

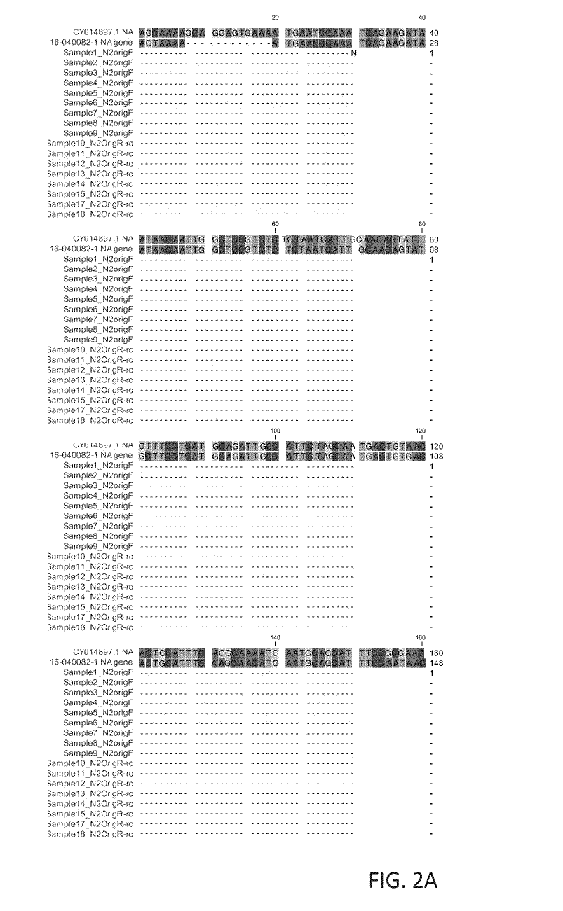

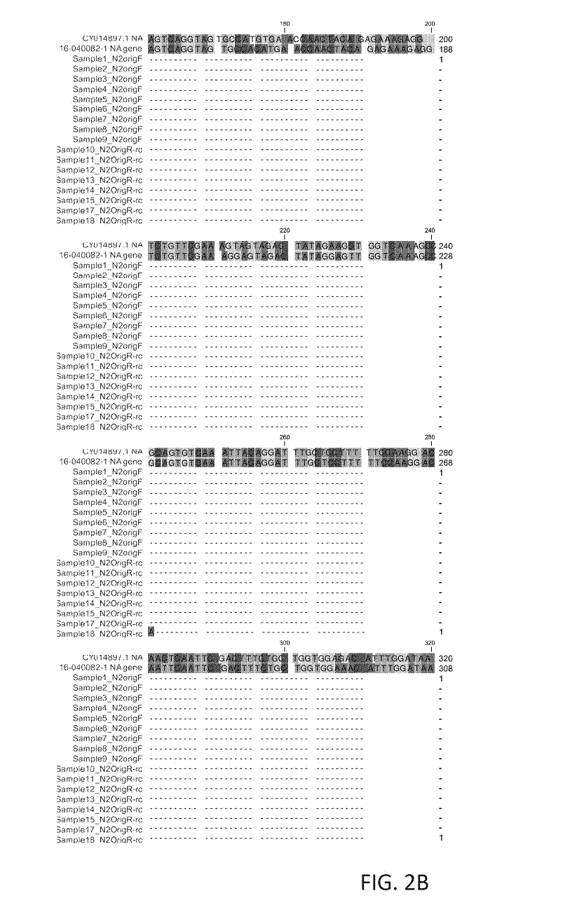

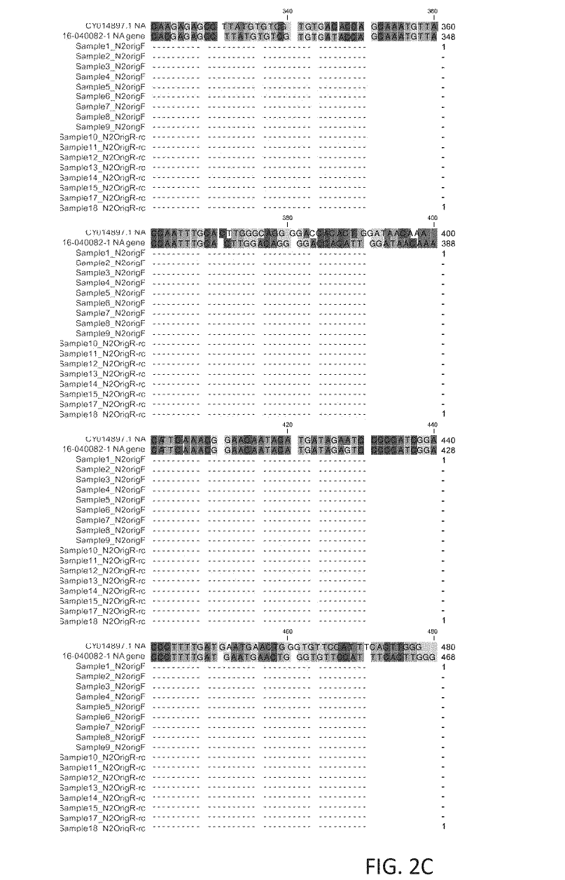

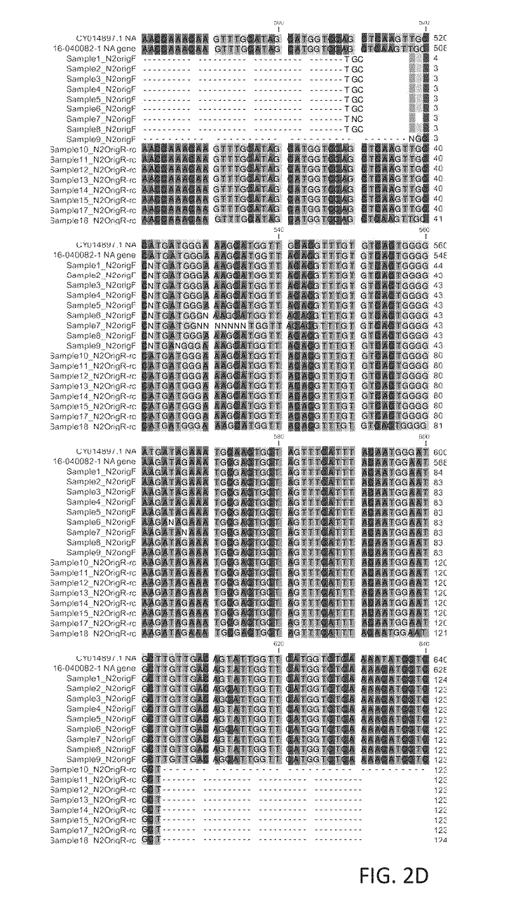

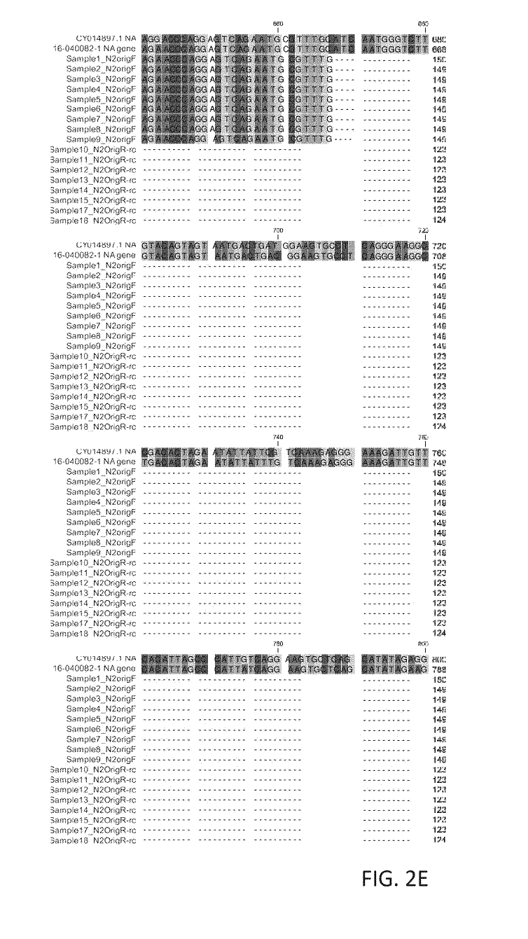

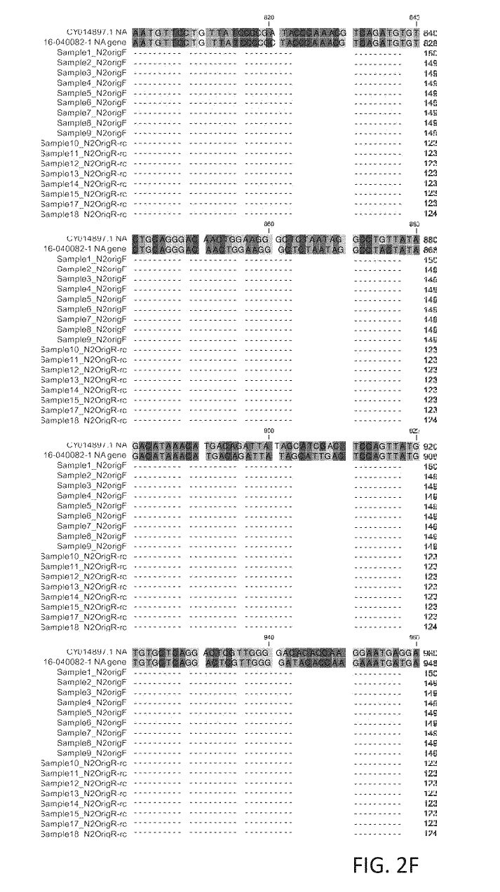

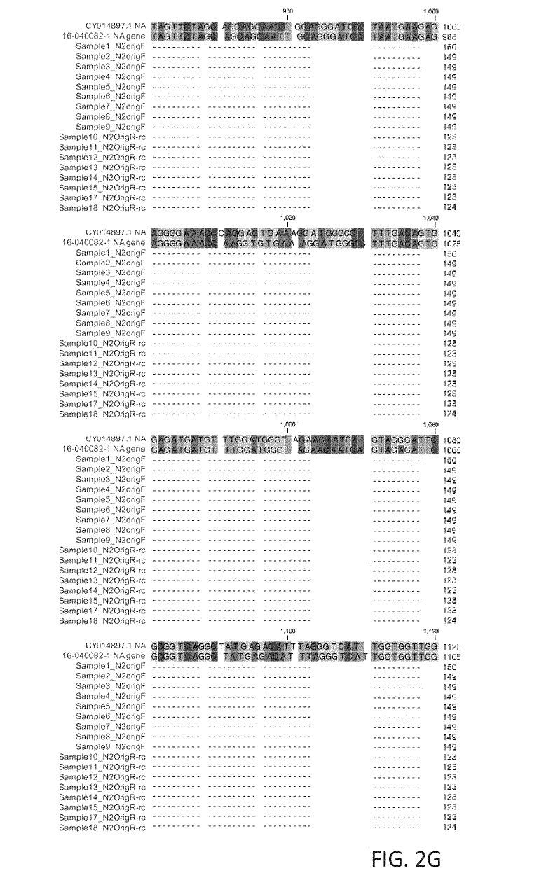

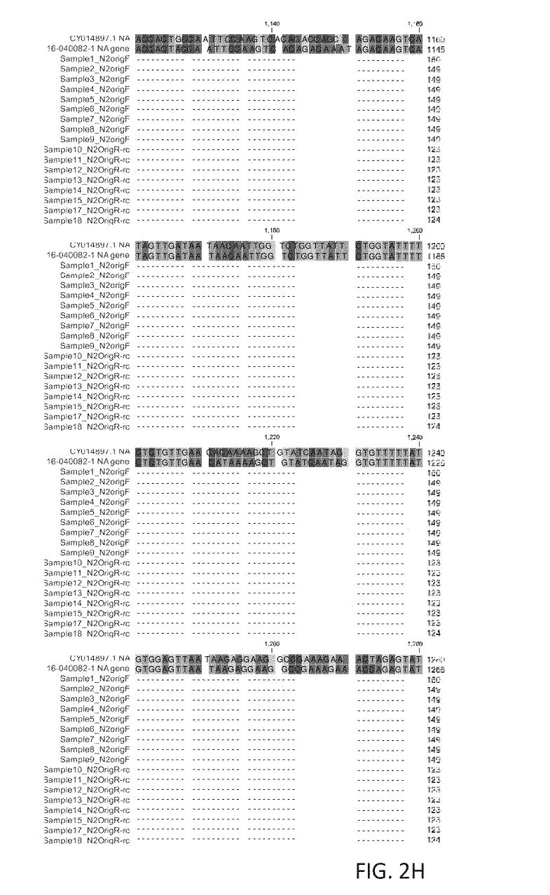

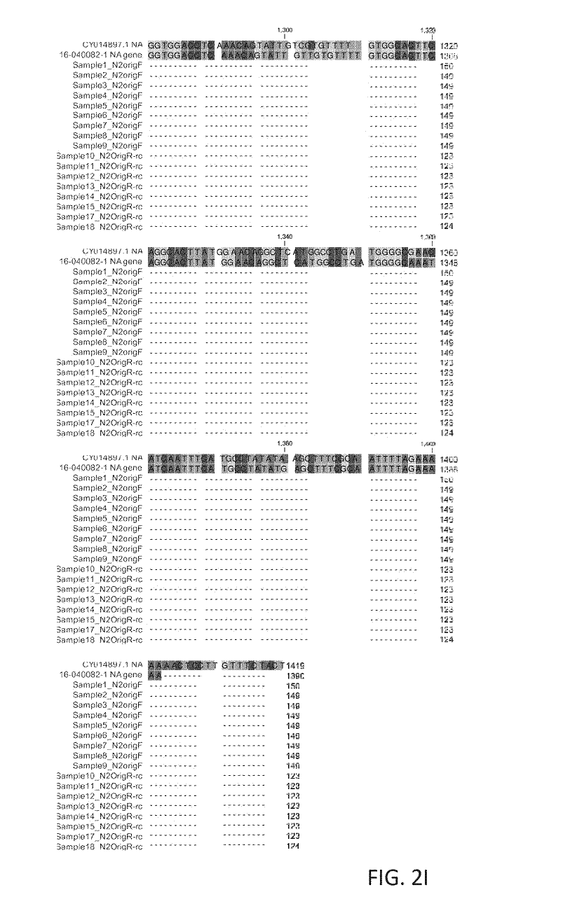

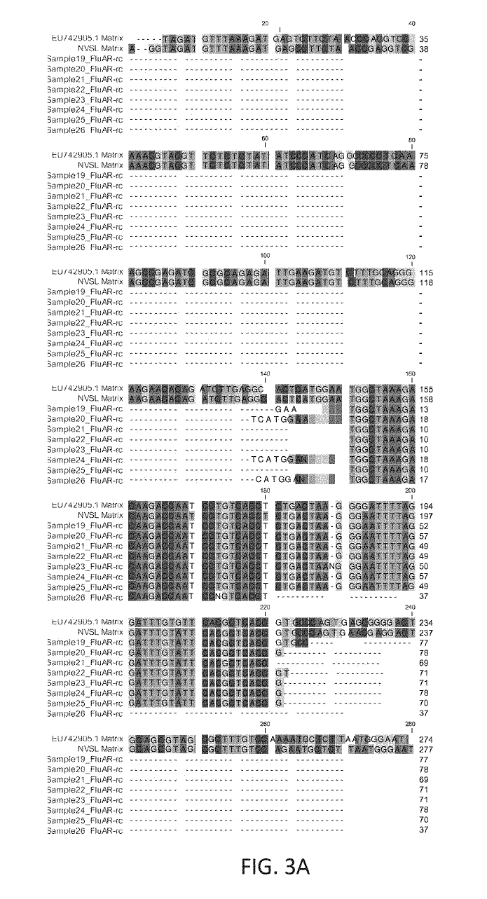

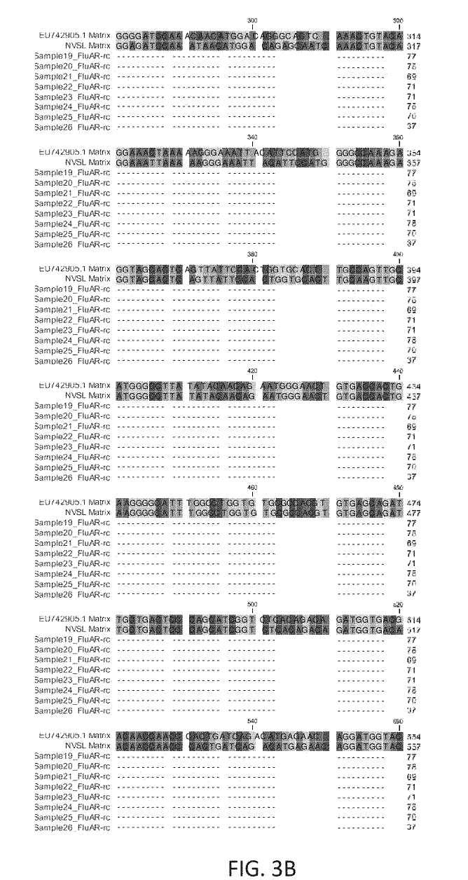

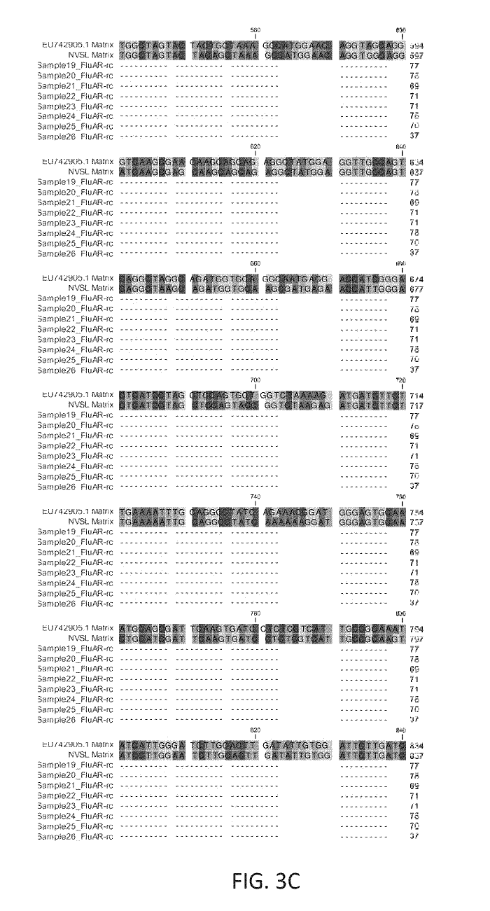

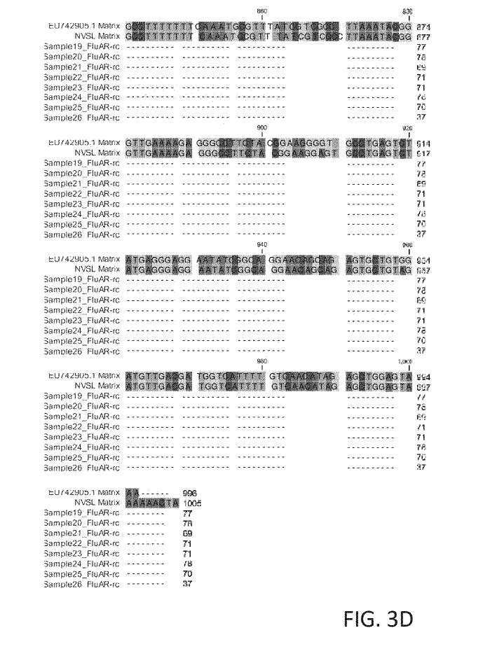

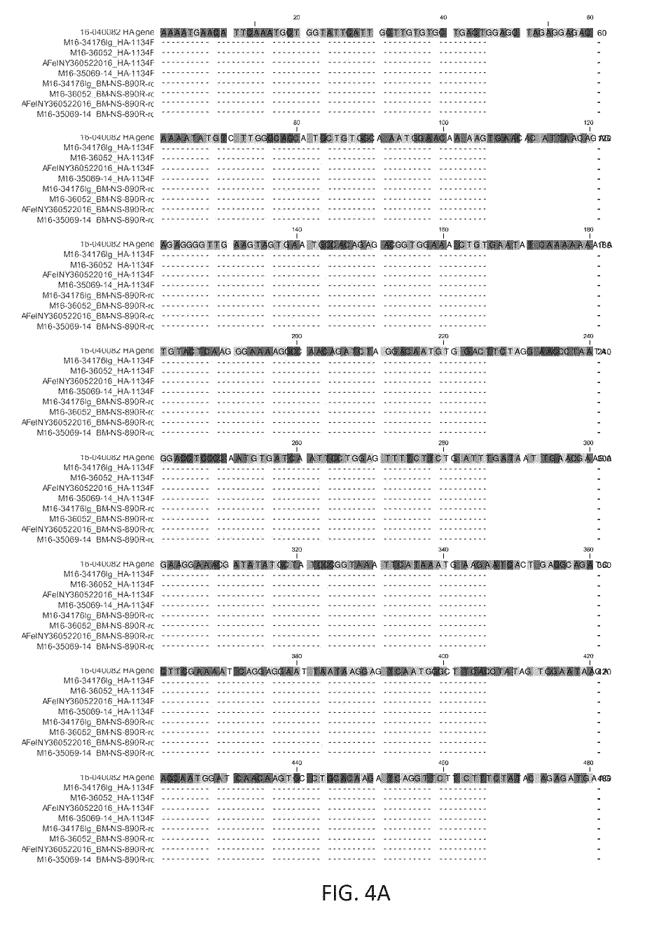

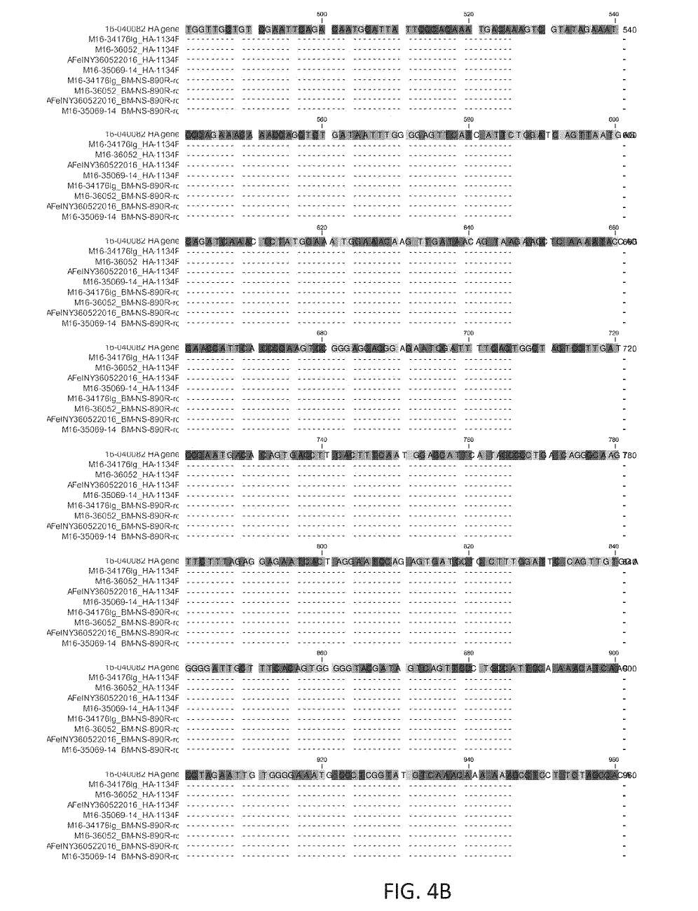

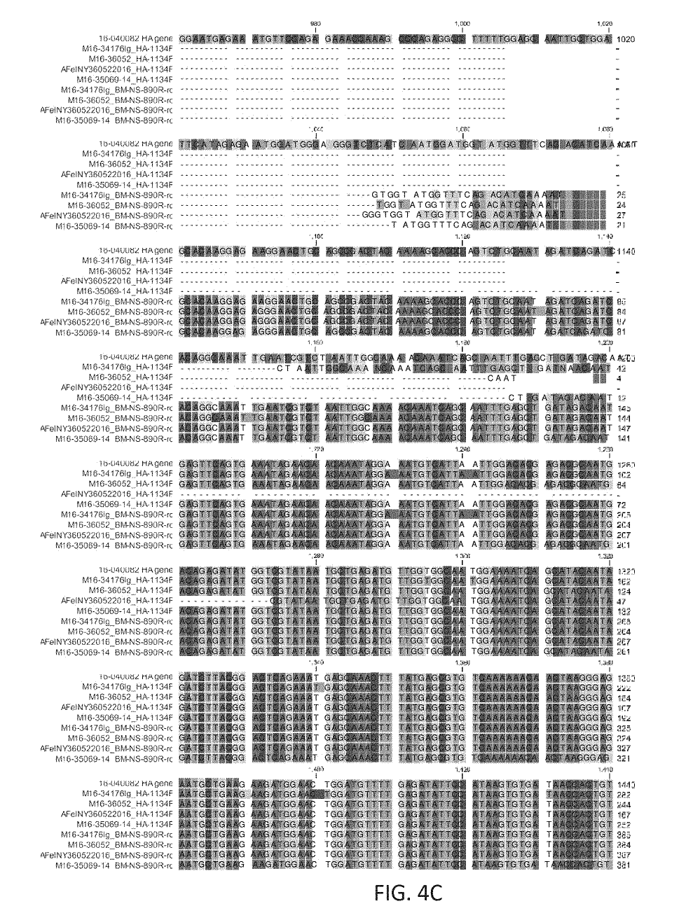

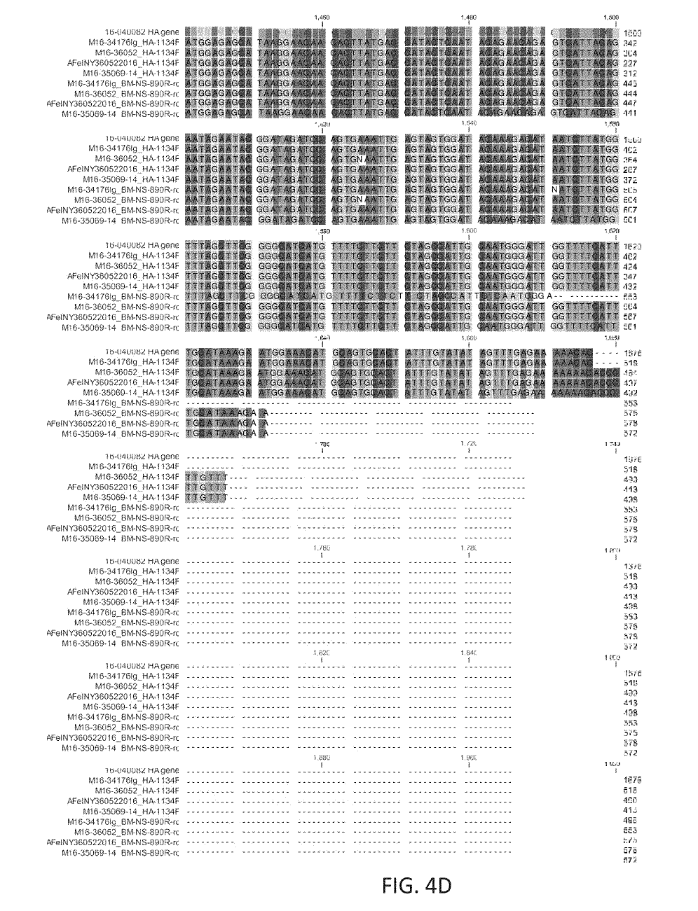

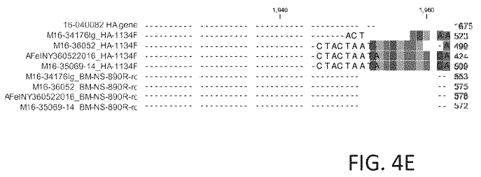

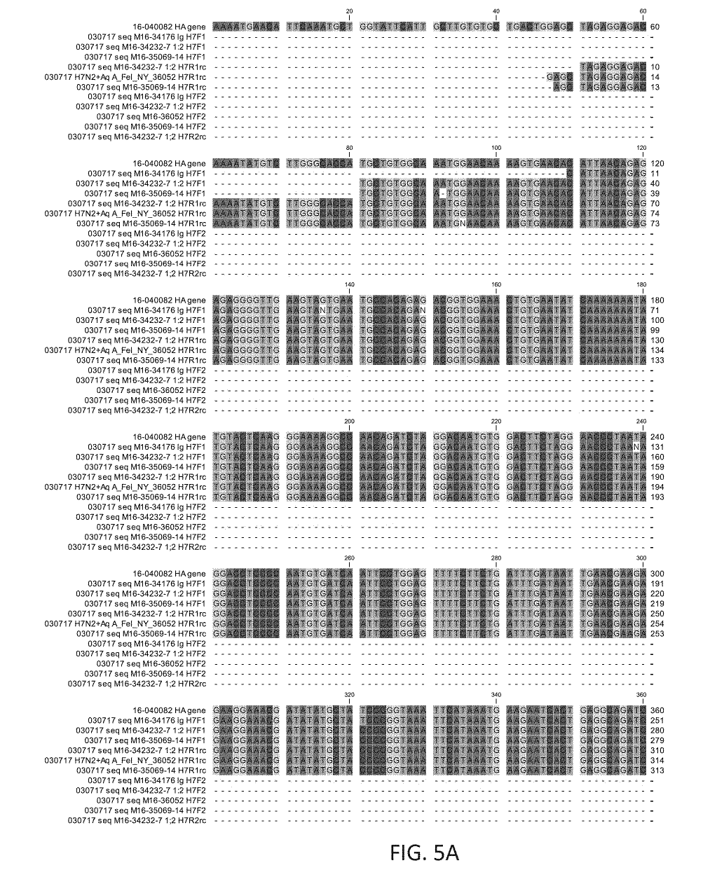

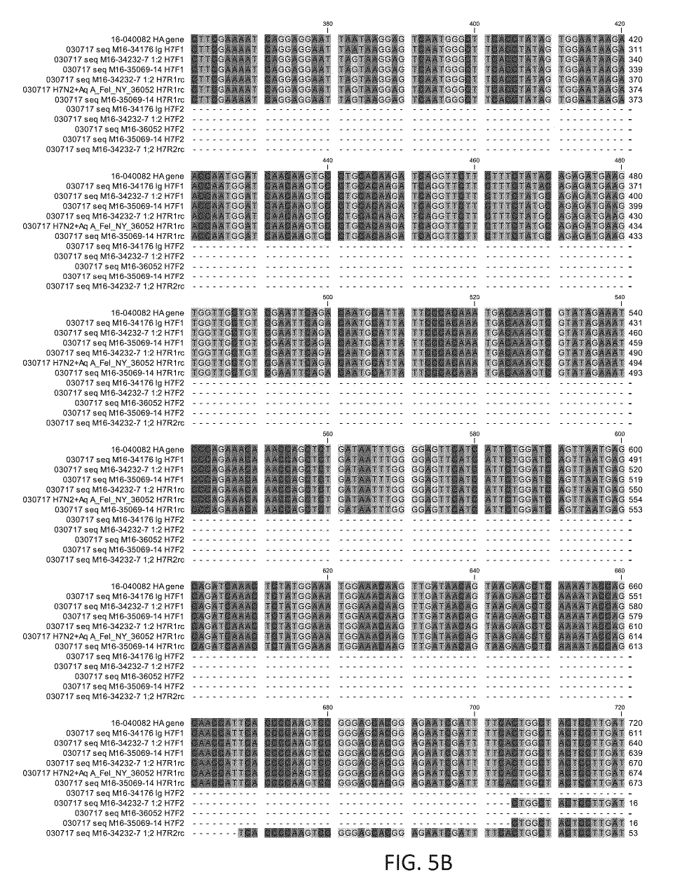

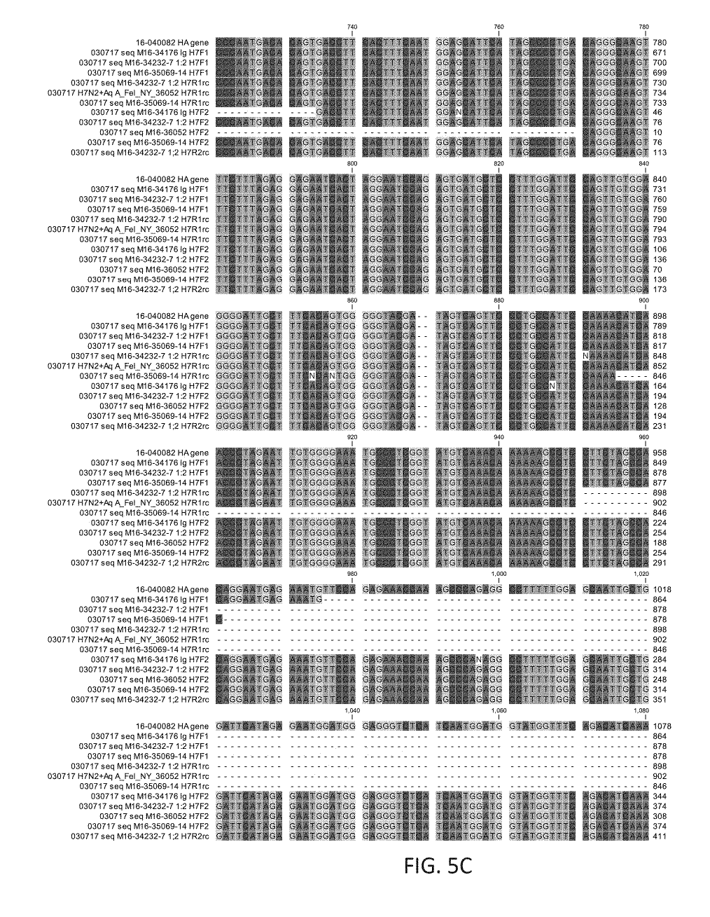

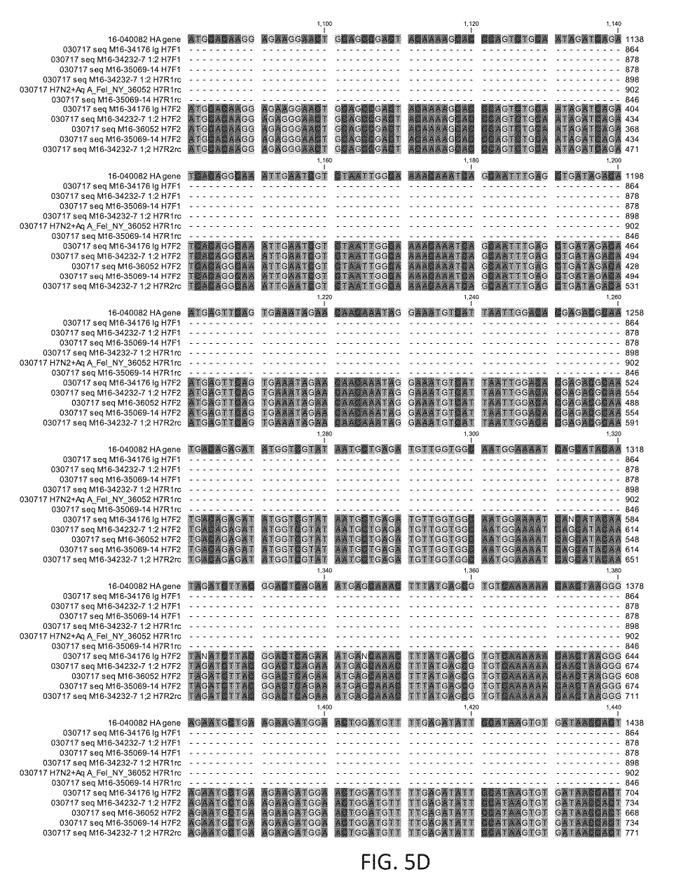

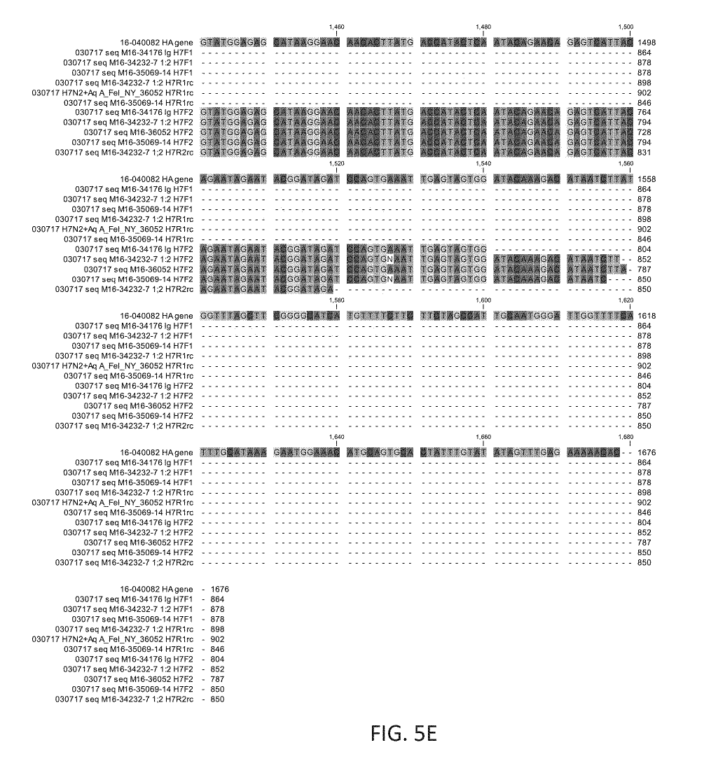

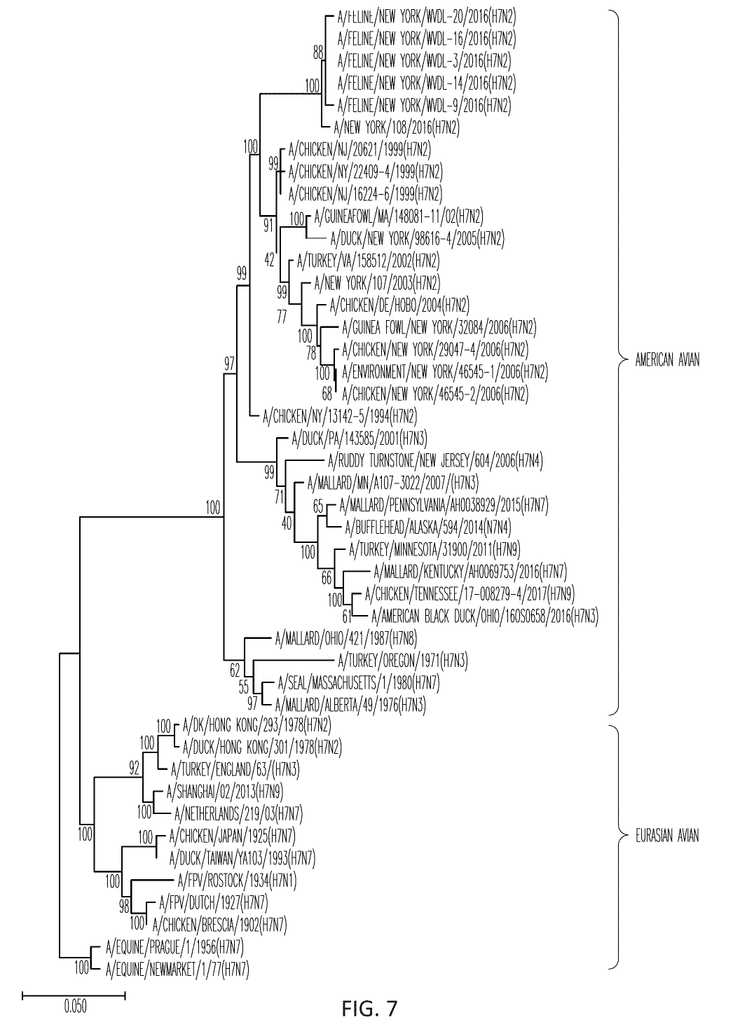

Swabs (collected on the same day) were obtained from 5 cats that experienced influenza-like symptoms during the outbreak at an animal shelter in New York, N.Y., in December 2016. After inoculation of these samples into MDCK cells, we isolated 5 pleomorphic influenza A viruses of the H7N2 subtype (Table 1; and see online Technical Appendix, FIG. 2). The HA consensus sequences of the 5 isolates (established by Sanger sequence analysis) displayed >99.9% similarity at the nucleotide level (Table 1). Phylogenetic analyses demonstrated that the 8 viral RNA segments of the 5 feline H7N2 viruses are most closely related to poultry influenza A(H7N2) viruses detected in the New York area in the late 1990s through early 2000s (FIG. 7; online Technical Appendix FIGS. 3-9), suggesting that the 2016 feline H7N2 virus isolates descended from viruses that circulated more than a decade ago in the northeastern United States.

TABLE 1
Characterization of a Feline Influenza A(H7N2) Virus
Amino acid differences among feline influenza A(H7N2) virus isolates,
New York, NY, USA*

| | Amino acid positions | | | | | |
|---|---|---|---|---|---|---|
| | in the viral proteins | | | | | |
| | PB2 | PB1-F2 | PA | NA | | NS2 |
| Virus | 448 | 42 | 57 | 40 | 62 | 74 |
| A/feline/New York/WVDL-3/2016 | S | C | Q | Y | C | D |
| A/feline/New York/WVDL-9/2016 | N | Y | R | H | C | E |
| A/feline/New York/WVDL-14/ | S | C | Q | Y | C | E |
| 2016 | | | | | | |
| A/feline/New York/WVDL-16/ | S | C | Q | Y | F | E |
| 2016 | | | | | | |
| A/feline/New York/WVDL-20/ | S | C | Q | Y | C | D |
| 2016 | | | | | | |

*Consensus sequences among the 5 H7N2 subtype viruses are snown in bold.
Amino acids: C, cysteine; D, aspartic acid; E, glutamic acid; F, phenylalanine; H, histidine; L, leucine; N, asparagine; Q, glutamine; R, arginine; S, serine; Y, tyrosine.
Viral proteins: NA, neuraminidase; NS, nonstructural protein; PA polymerase acidic; PB, polymerase basic.

The HA protein of the 2016 feline H7N2 subtype virus encodes a single arginine residue at the hemagglutinin cleavage site (PEKPKPR↓G; the arrow indicates the cleavage site that creates the HA1 and HA2 subunits), indicative of low pathogenicity in chickens. Antigenically, A/feline/New York/WVDL-14/2016 (A/feline/NY/16) differs from other, closely related H7 viruses (online Technical Appendix Table 1); for example, its HA deviates by 27 aa from the closely related A/chicken/NY/22409-4/1999 H A. The neuraminidase (NA) and ion channel (M2) proteins of the H7N2 viruses do not encode amino acids that confer resistance to neuraminidase or ion channel inhibitors. Inspection of the remaining feline H7N2 viral proteins revealed an absence of the most prominent amino acid changes known to facilitate adaptation to mammals, such as PB2-627K, These data thus suggest the 2016 feline 117N2 subtype viruses are avian-derived influenza viruses of low pathogenicity in avian and mammalian species.

### Replication of Feline and Avian H7N2 Subtype Viruses in Cultured Cells

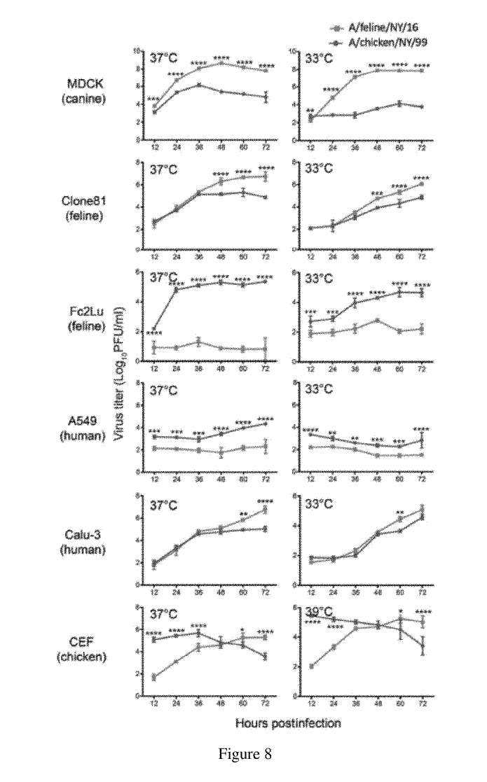

To characterize the replicative ability of the 2016 feline H7N2 viruses in cultured cells, A/feline/NY/16 (which encodes the consensus amino acid sequence of the 5 isolates) was compared with a closely related 1999 avian influenza virus, A/chicken/NY/22409-4/1999 (H7N2, A/chicken/NY/99), which was isolated from a chicken in a live-bird market in New York state in 1999 (Spackman et al., 2003). There are a total of 97 aa differences between A/feline/NY/16 and A/chicken/NY/99 viruses (12 aa differences in polymerase basic 2 (PB2), 7 in polymerase basic 1 (PB1), 12 in polymerase acidic (PA), 27 in hemagglutinin (HA), 8 in nucleoprotein (NP), 11 in neuraminidase (NA), 7 in matrix protein 1 (M1), 4 in matrix protein 2 (M2), and 9 in nonstructural protein 1 (NS1). Canine, human, feline, and chicken cells were infected at a multiplicity of infection of 0.005 at temperatures mimicking those of the upper and lower respiratory tract of the respective species (i.e., 37° C. and 39° C. for chicken cells; 33° C. and 37° C. for the remaining cells) (FIG. 8). In canine MDCK, feline Clone81, and human Calu-3 cells, A/feline/NY/16 replicated at least as efficiently as A/chicken/NY/99 virus, while both viruses replicated to low titers in human A549 cells. Of note, A/feline/NY/16 virus replicated less efficiently than A/chicken/NY/99 virus in feline lung Fc2Lu cells. In chicken embryo fibroblast cells, A/feline/NY/16 virus replicated more slowly than A/chicken/NY/99 virus at early time points and reached its highest titers at later time points. When virus growth at the 2 temperatures tested (i.e., 37° C. and 39° C. for chicken cells; 33° C. and 37° C. for the remaining cells) was compared, similar trends (for example, in MDCK cells A/feline/NY/16 replicated more efficiently than A/chicken/NY/99 at both temperatures tested) were observed.

### Replication and Pathogenicity of Feline and Avian H7N2 Subtype Viruses in Mice

To assess the replication of A/feline/NY/16 and A/chicken/NY/99 viruses in mice, 3 mice per group were inoculated intranasally with 10-fold dilutions of viruses, and their bodyweight and morbidity and mortality were monitored daily for 14 days. Mice infected with A/feline/NY/16 virus did not experience weight loss or signs of disease, whereas infection with 10⁶ PFU of A/chicken/NY/99 virus caused severe weight loss and required euthanasia (online Technical Appendix FIG. 10).

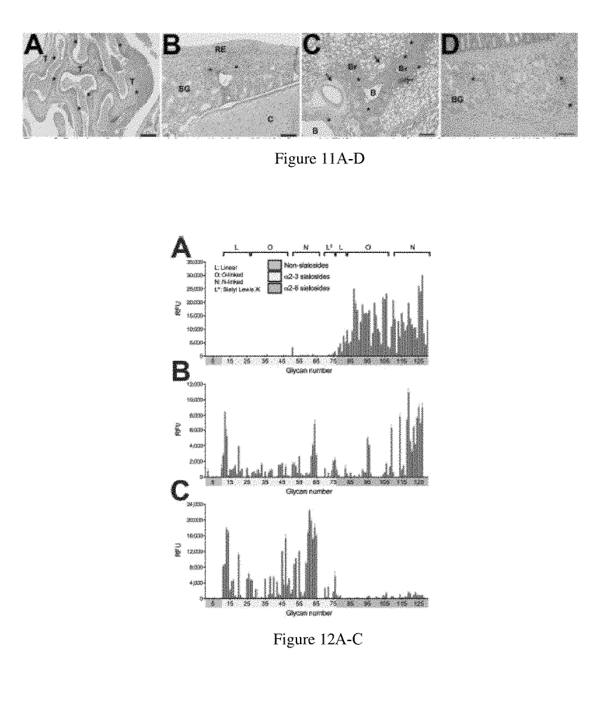

A/feline/NY/16 replicated efficiently in the nasal turbinates and less efficiently in the lungs of infected animals (online Technical Appendix FIG. 11); no virus was isolated from the other organs tested (i.e., brains, kidneys, livers, and spleens; data not shown). A/chicken/NY/99 replicated more efficiently in the lungs than in the nasal turbinates, consistent with immunohistochemistry analyses that detected A/feline/NY/16 virus antigens mainly in the upper respiratory organs of infected mice, whereas A/chicken/NY/99 virus antigens were detected more frequently in the lower respiratory organs (online Technical Appendix FIG. 12).

### Replication and Pathogenicity of Feline and Avian H7N2 Subtype Viruses in Ferrets

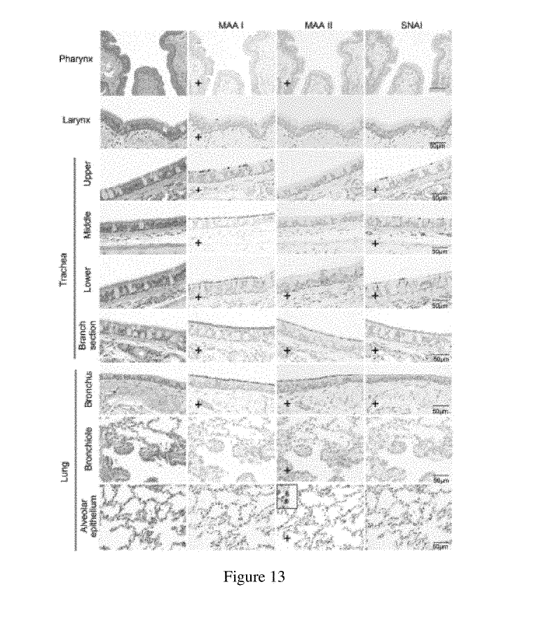

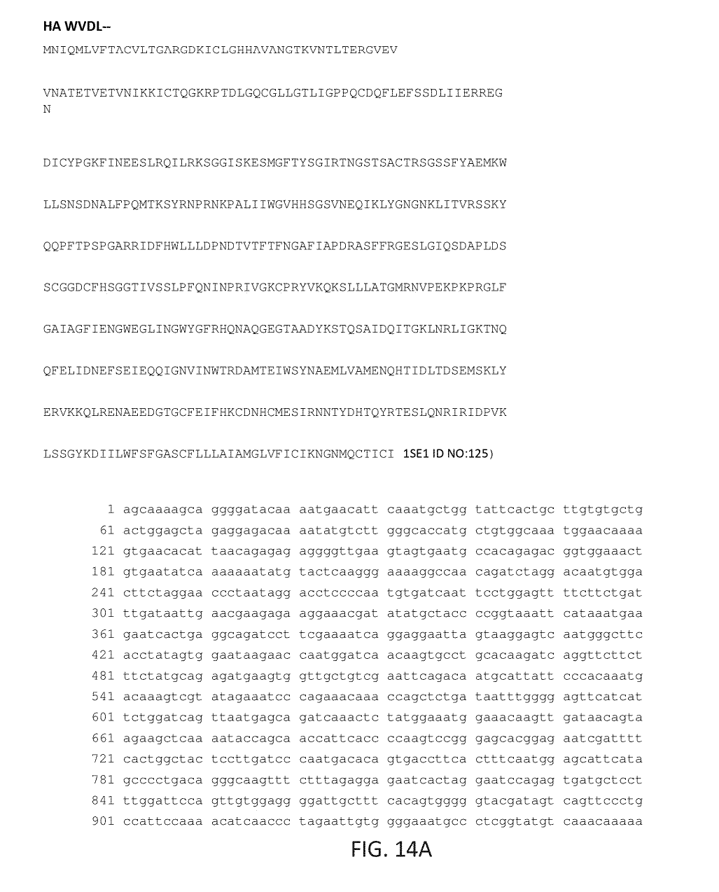

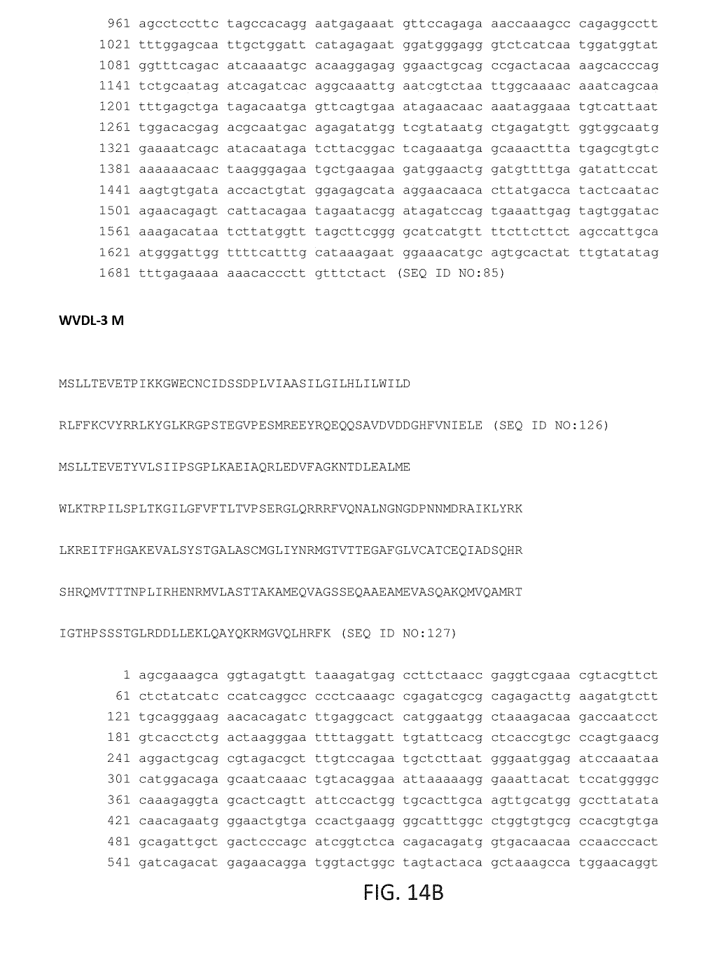

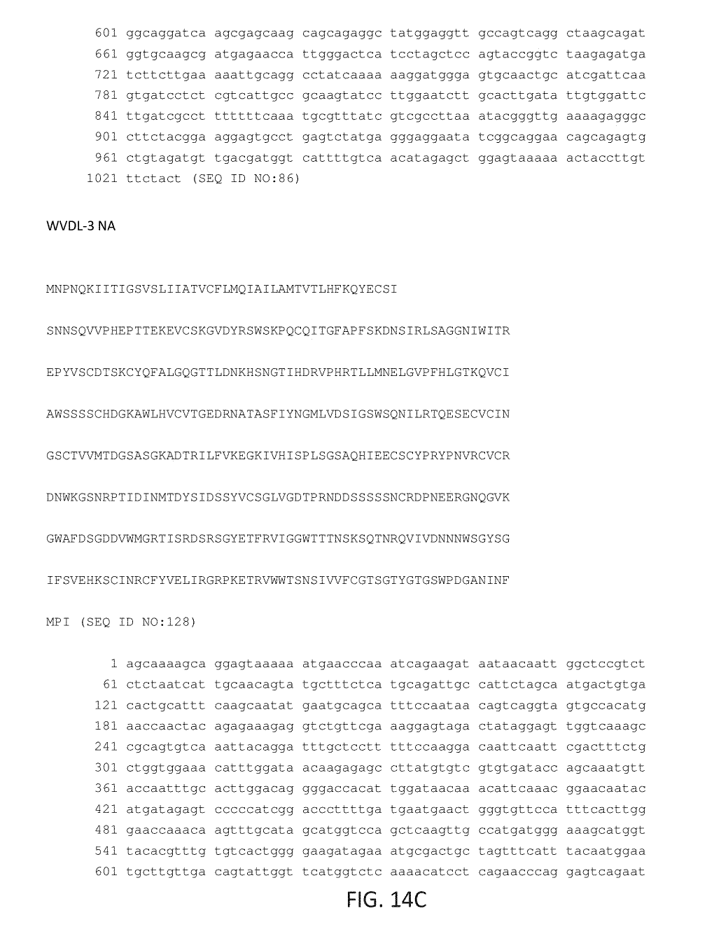

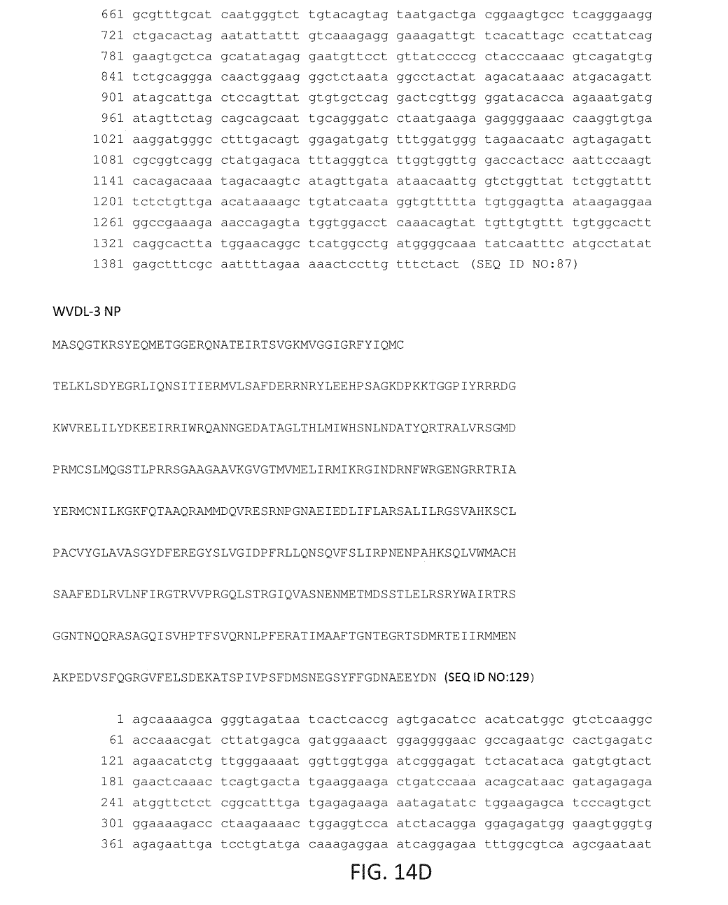

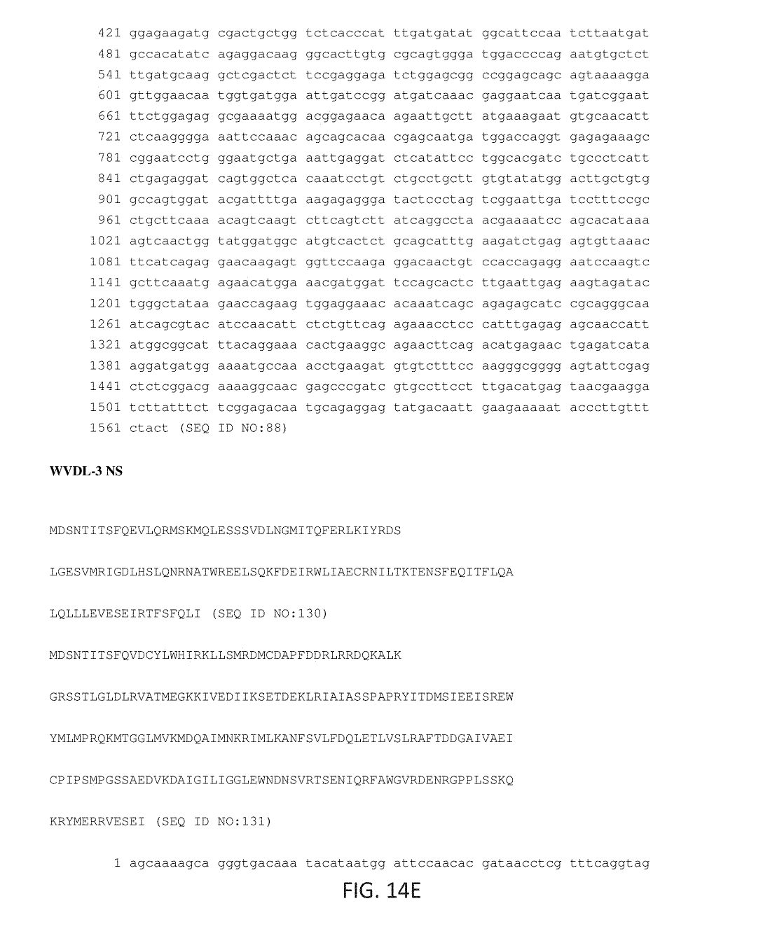

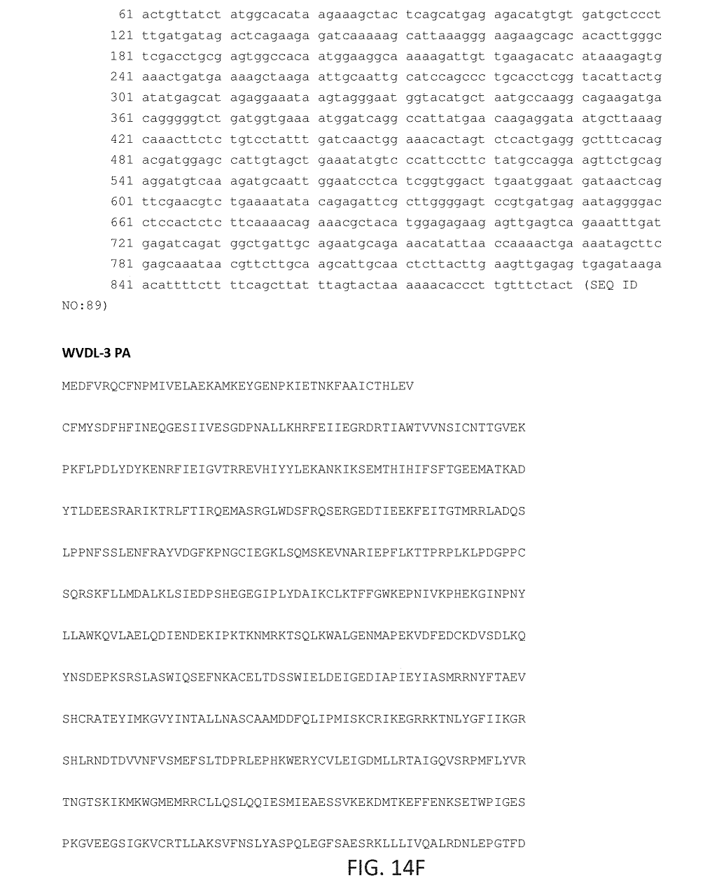

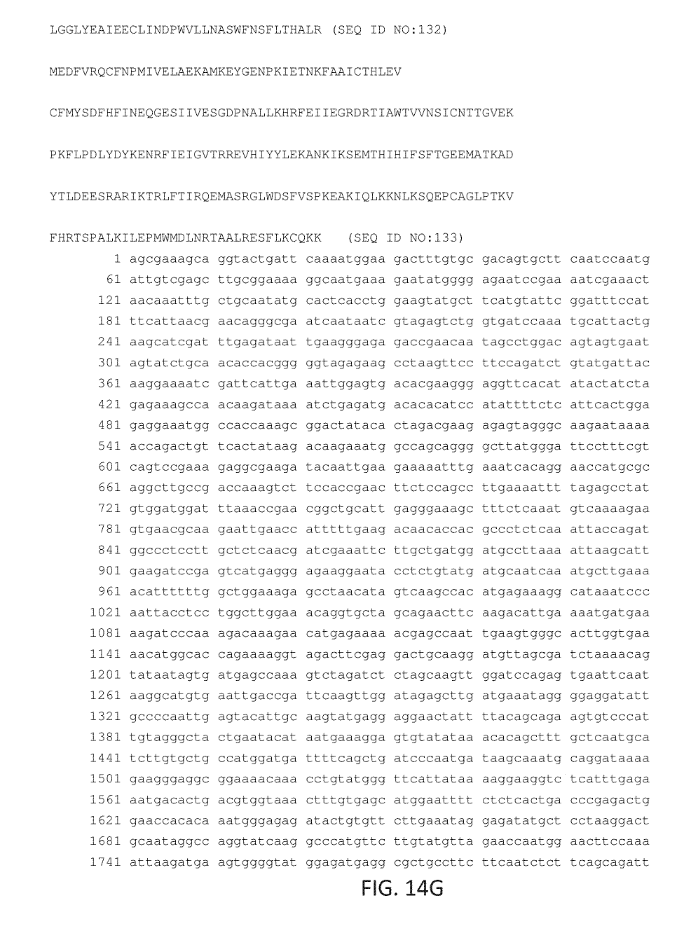

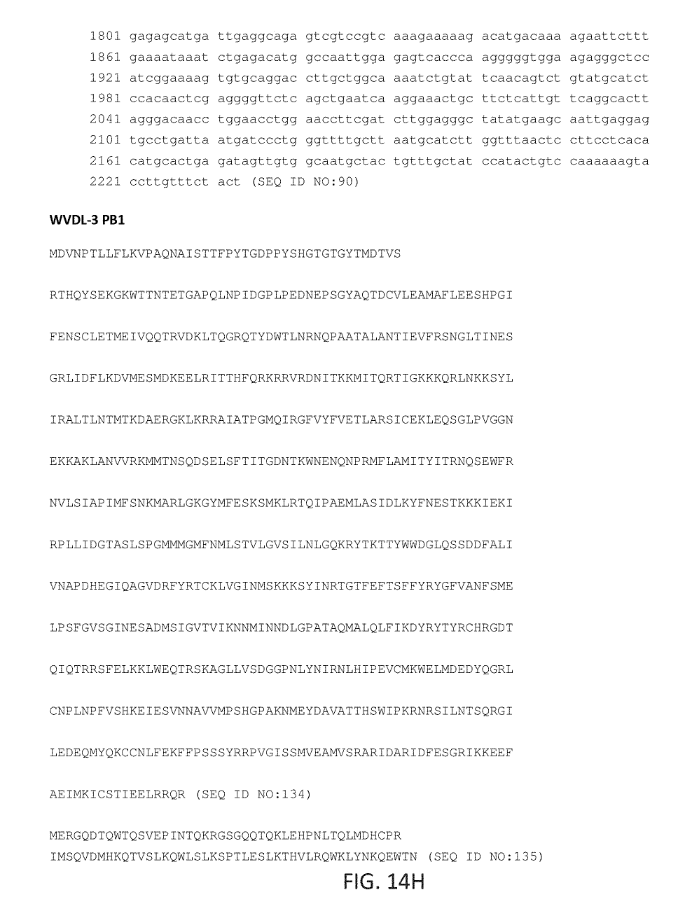

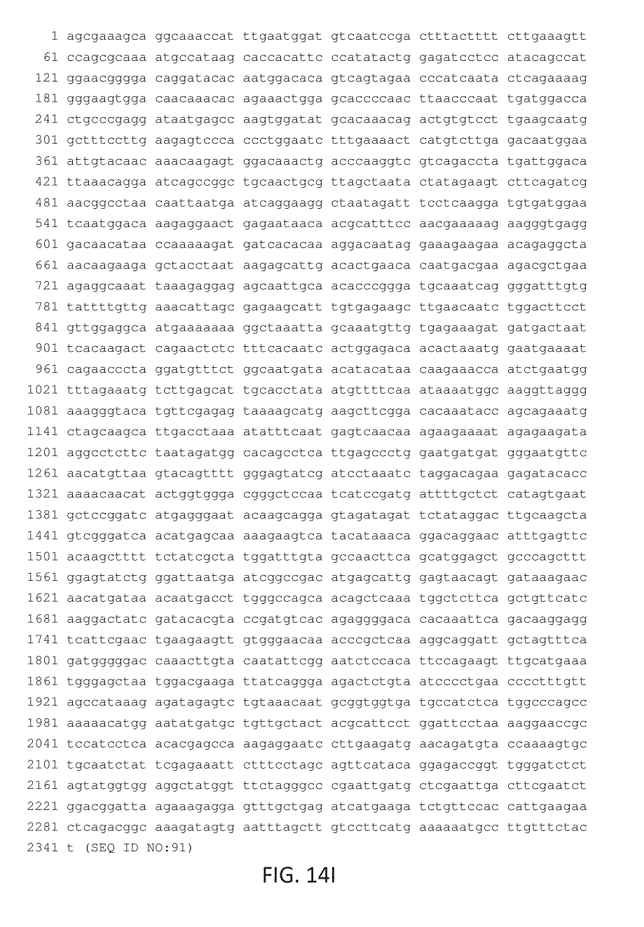

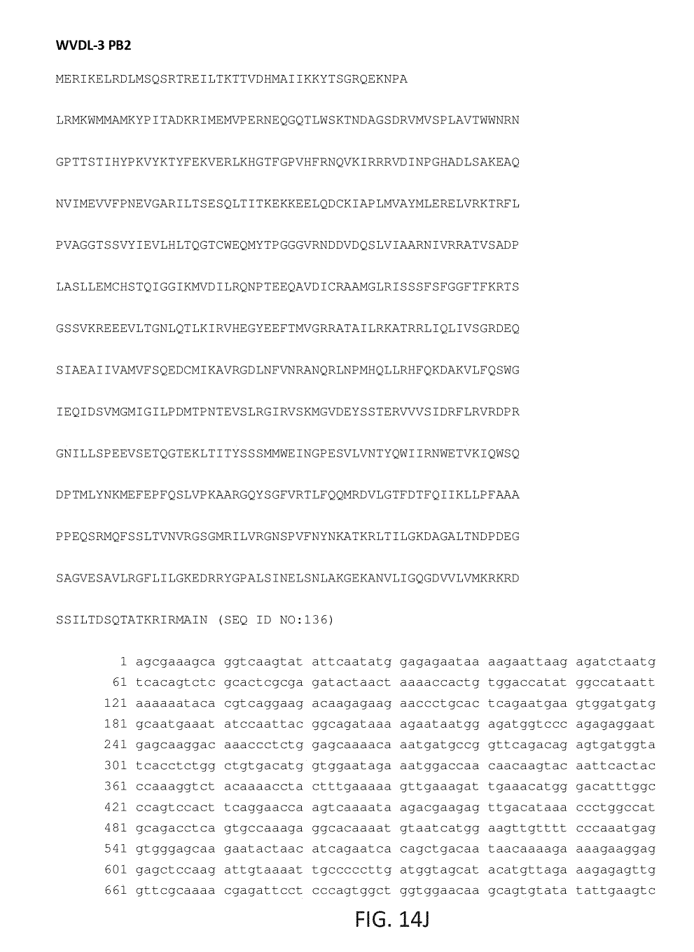

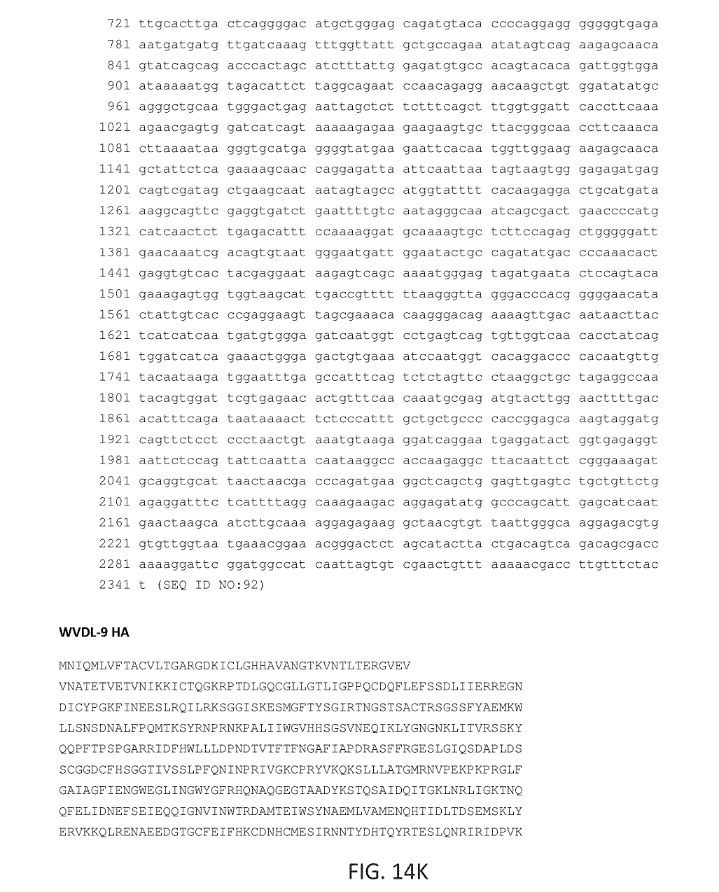

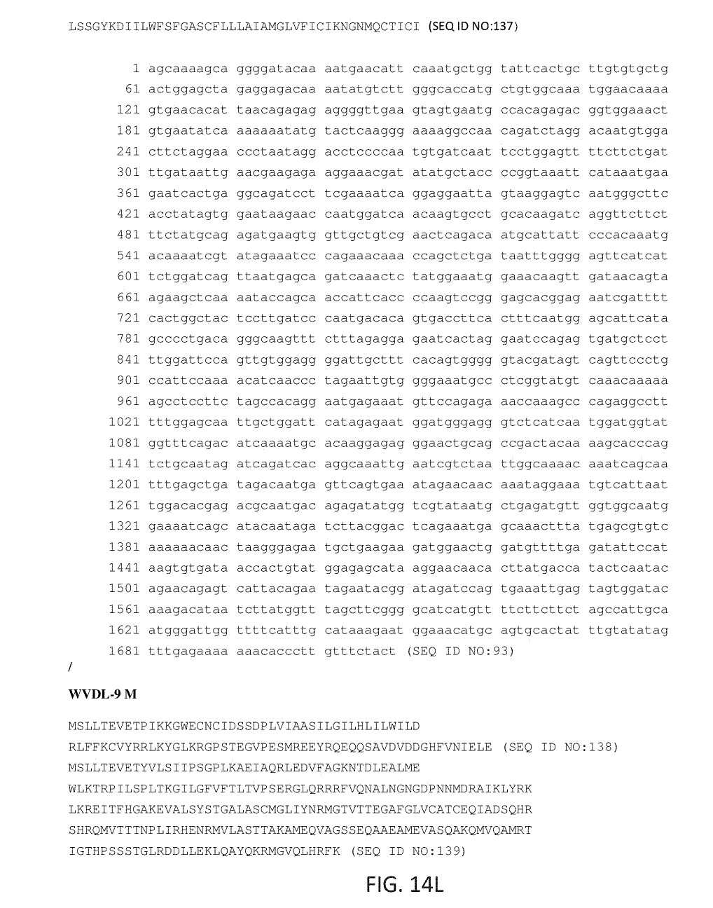

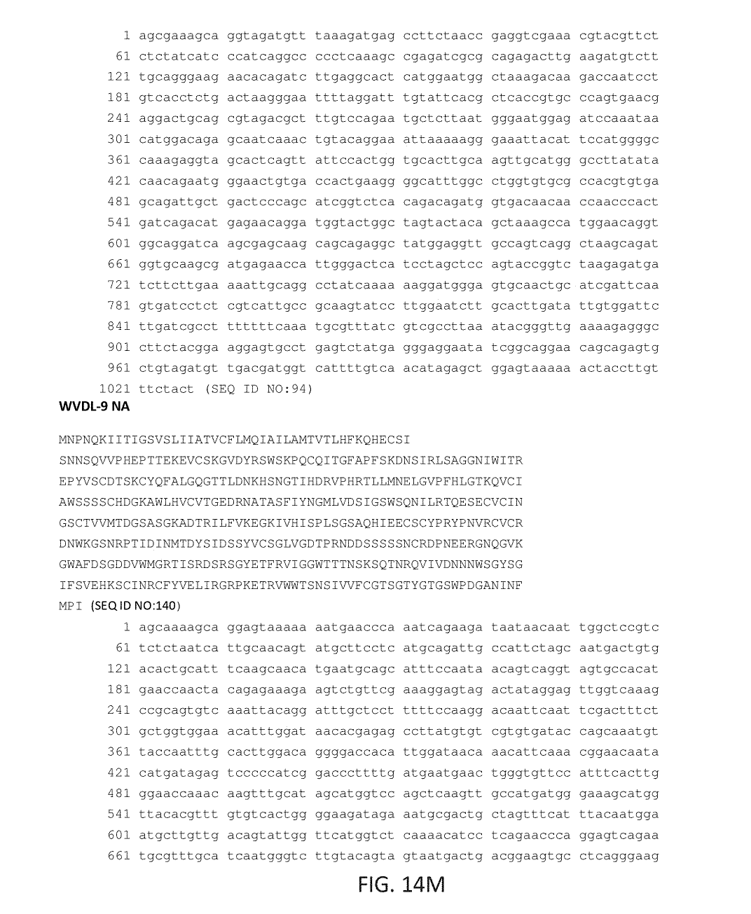

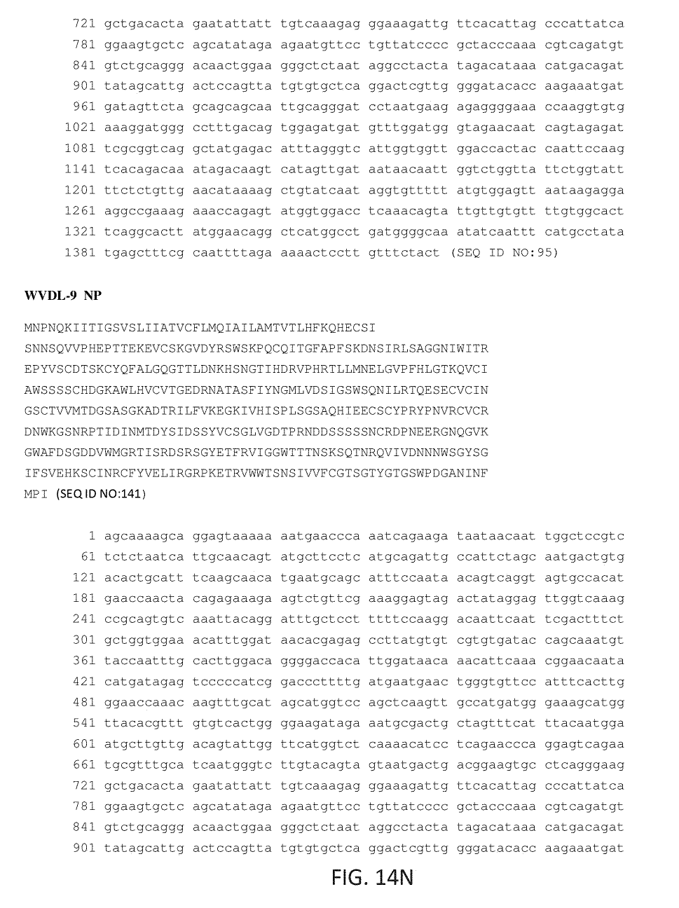

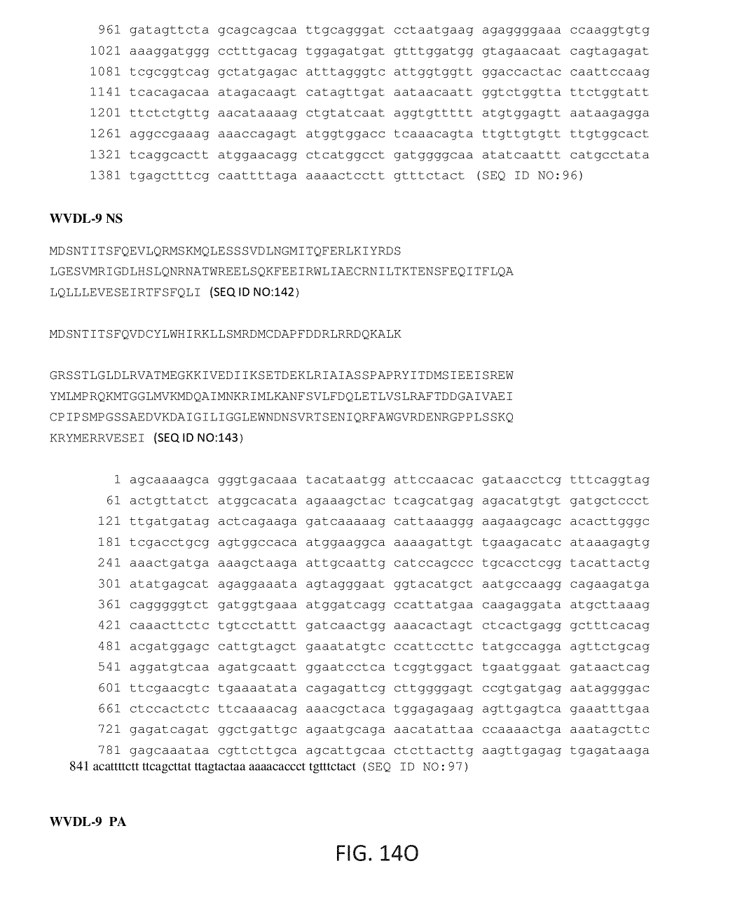

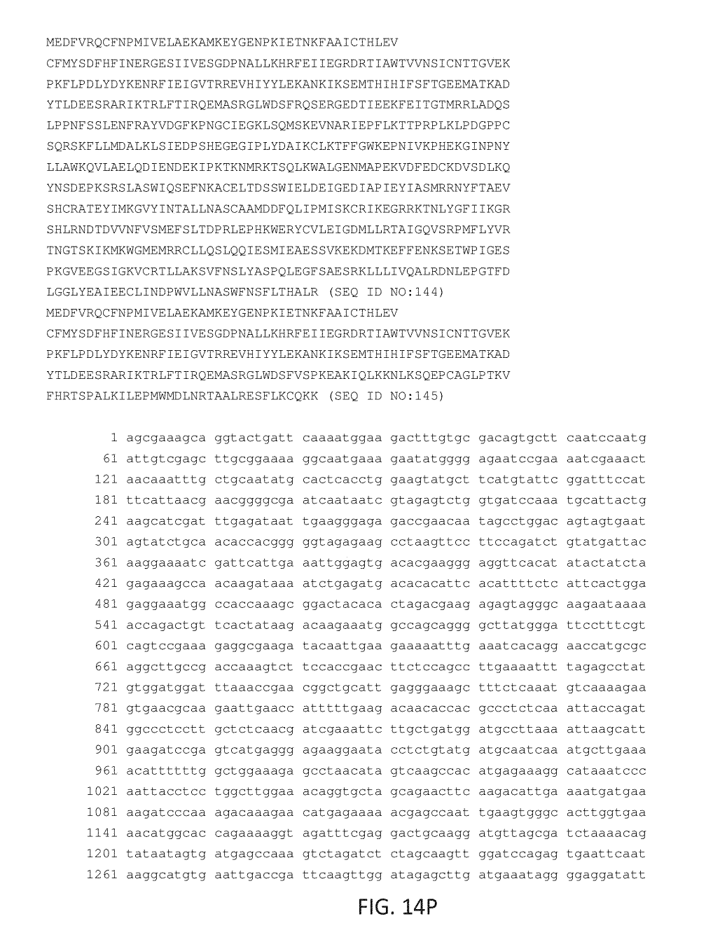

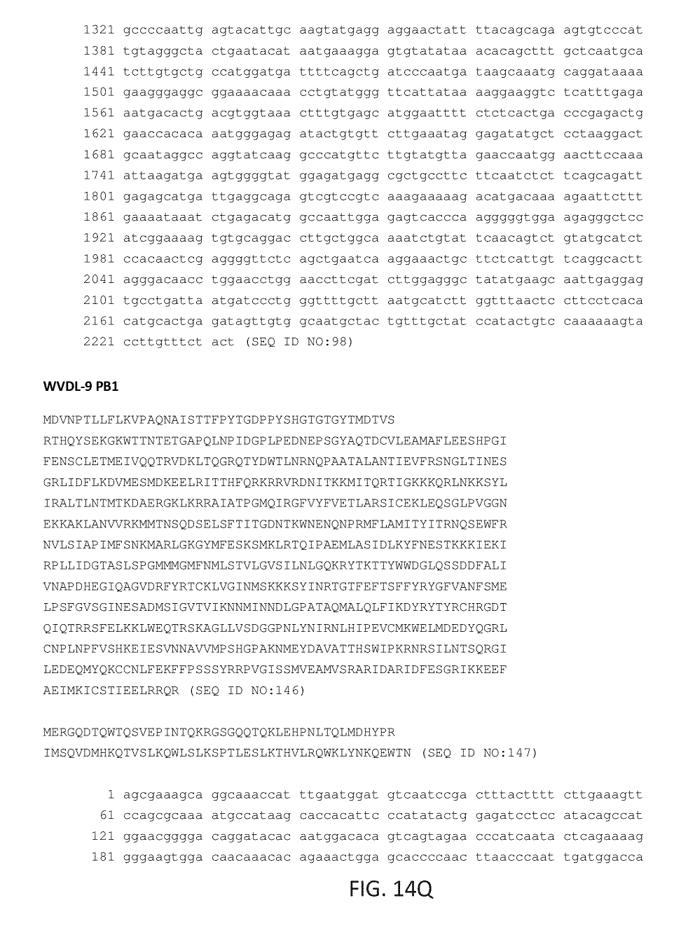

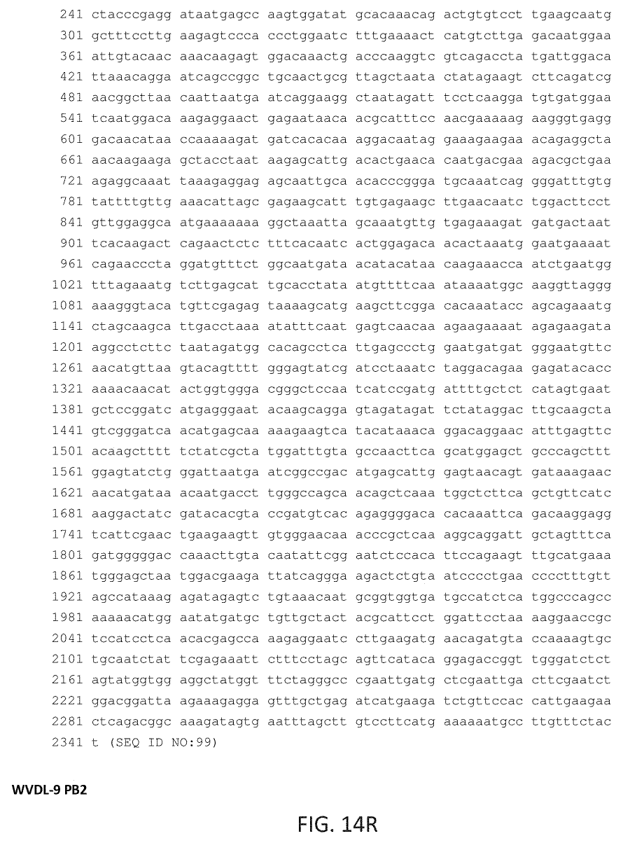

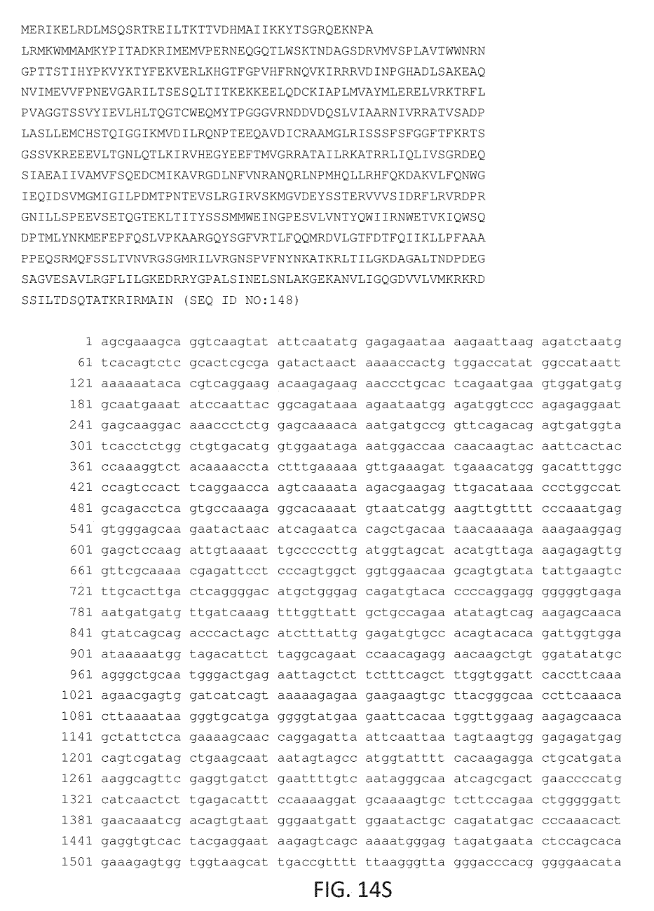

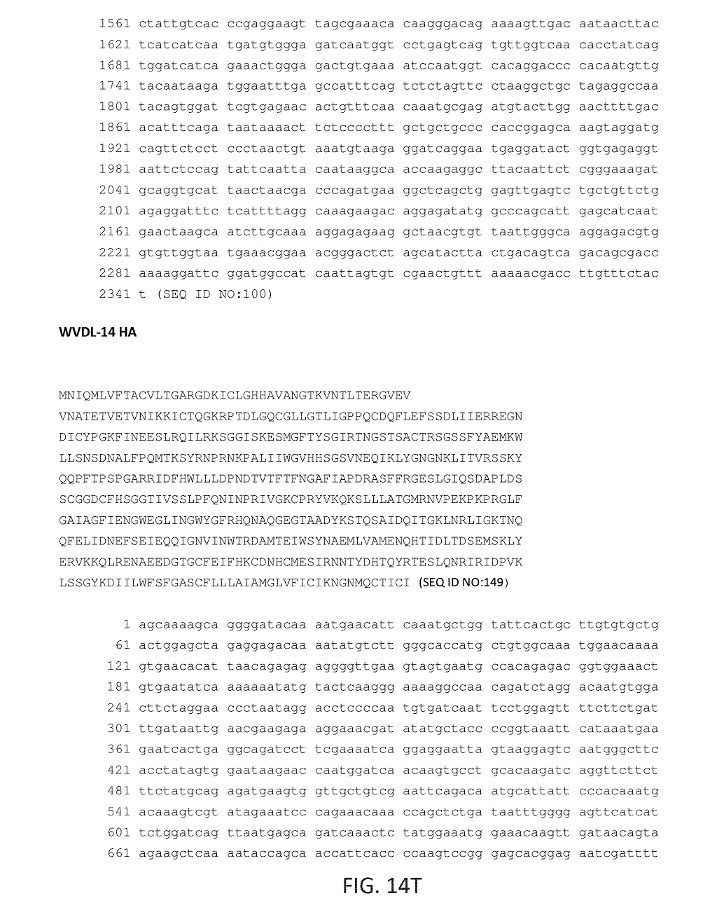

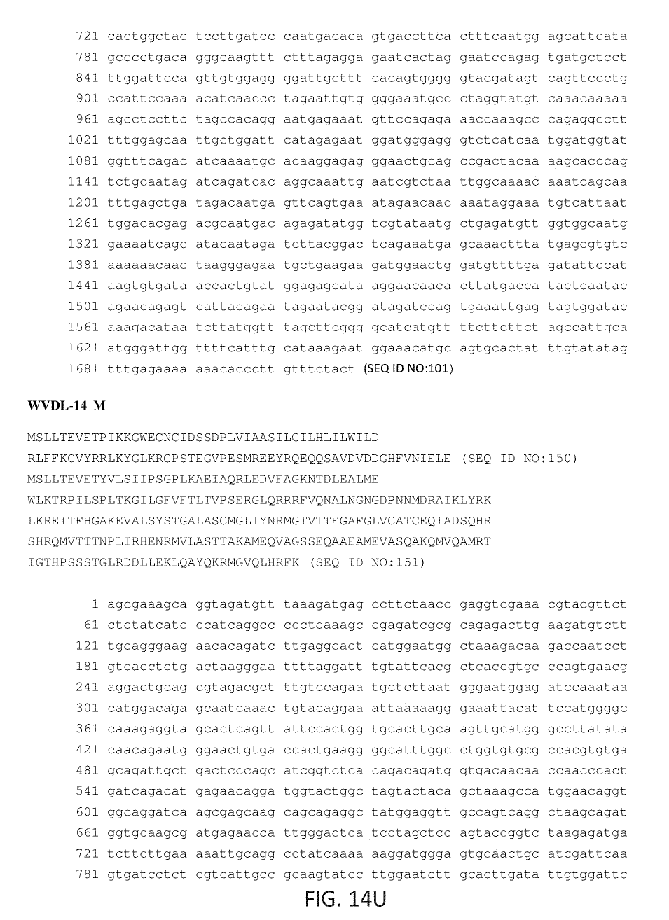

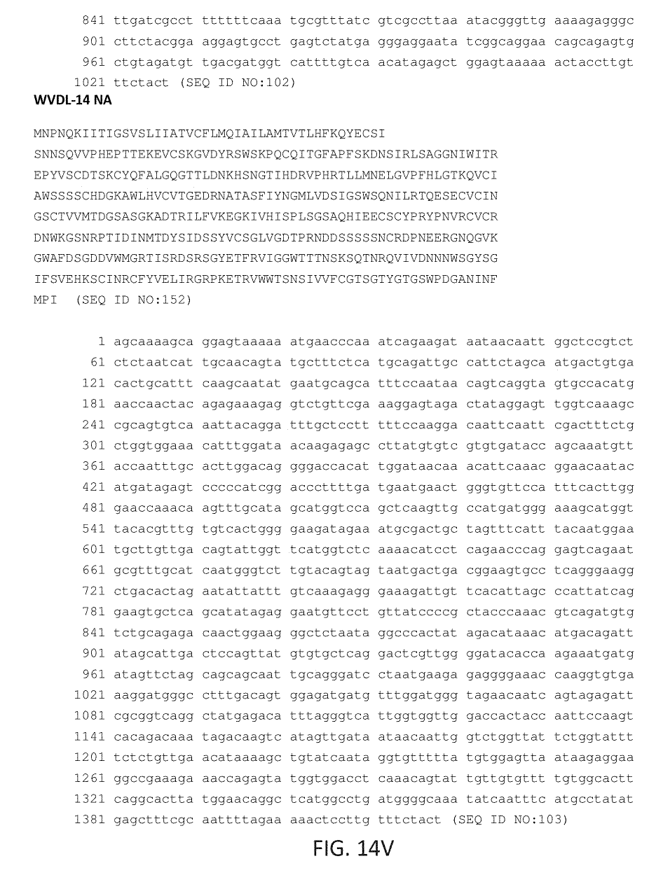

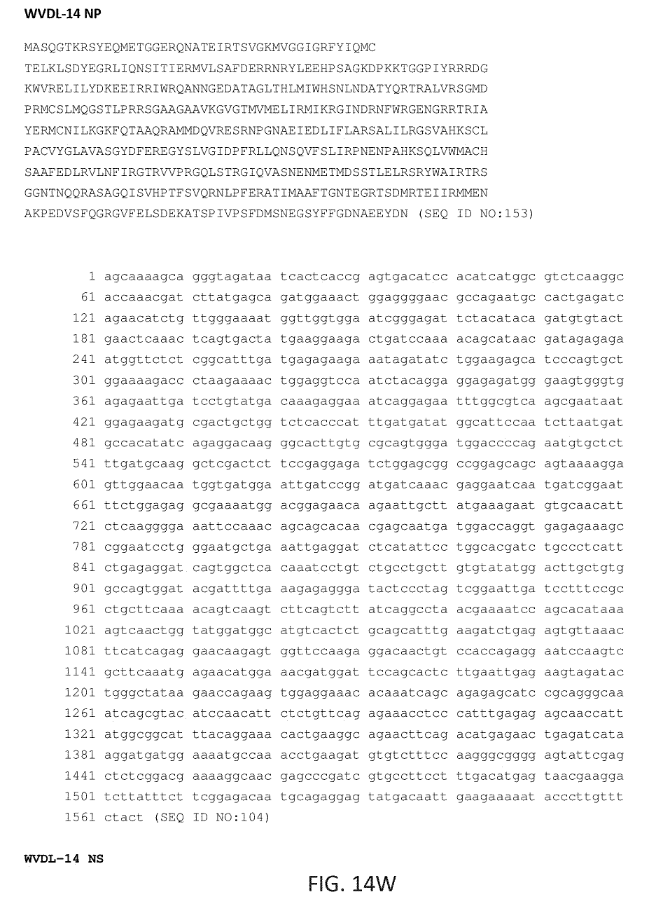

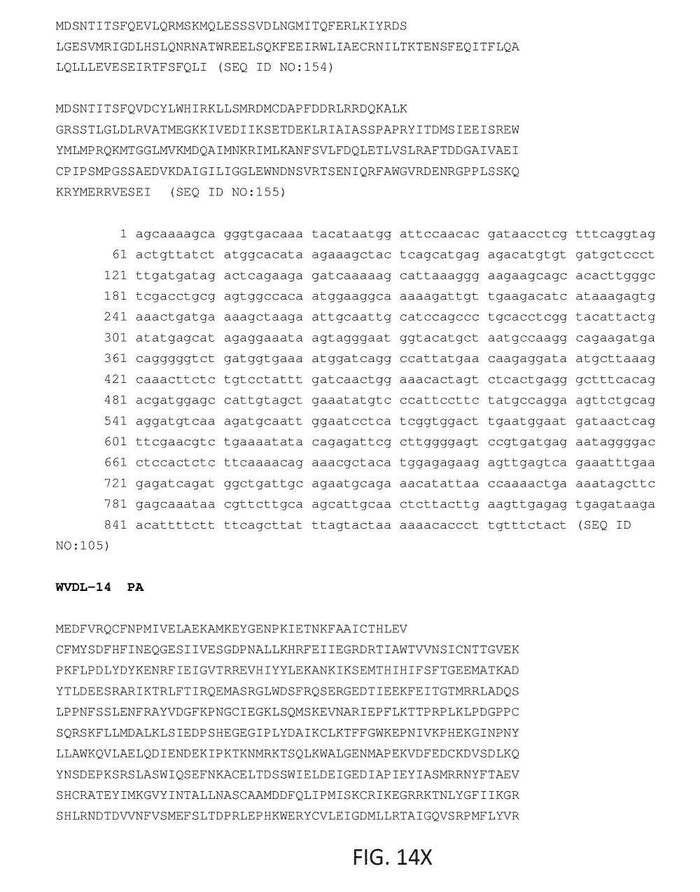

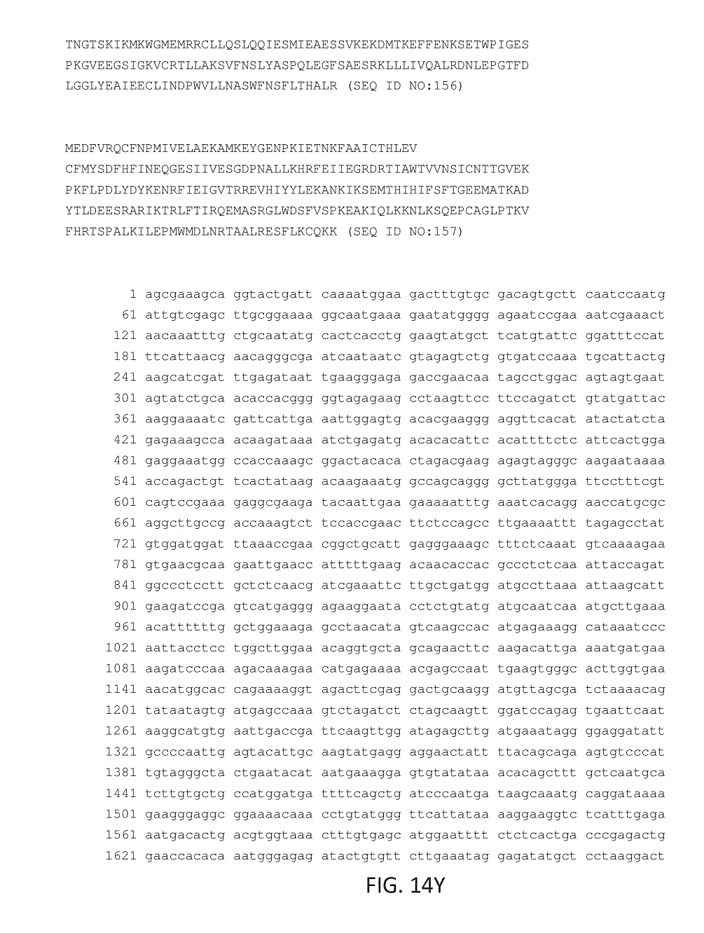

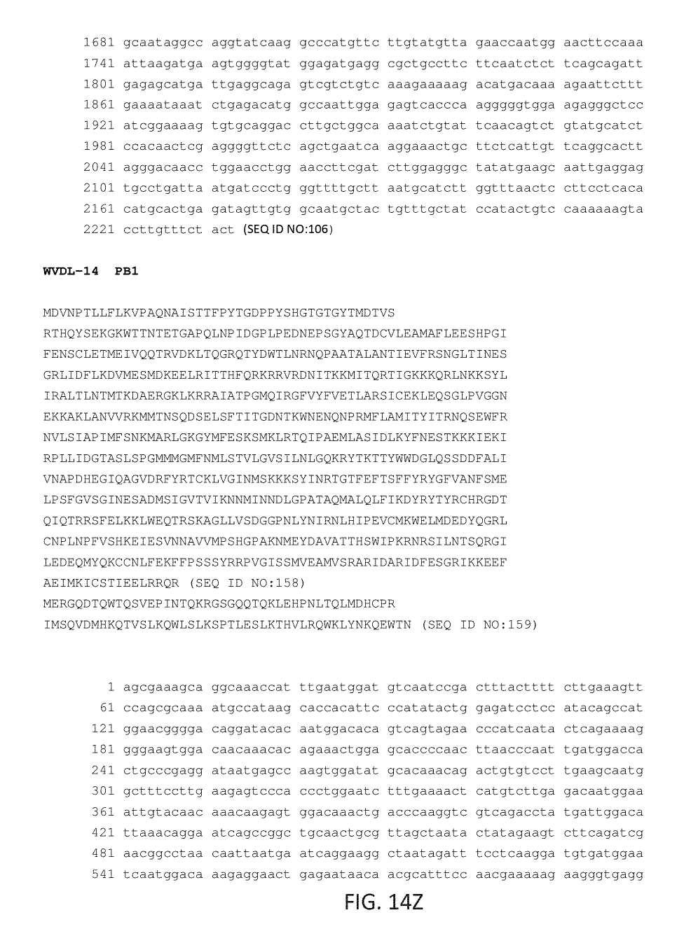

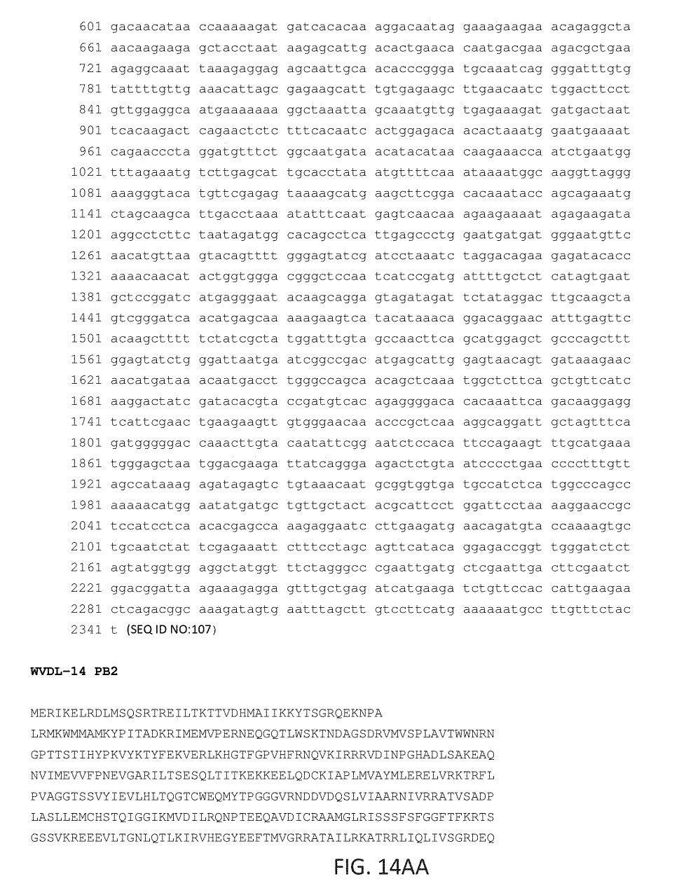

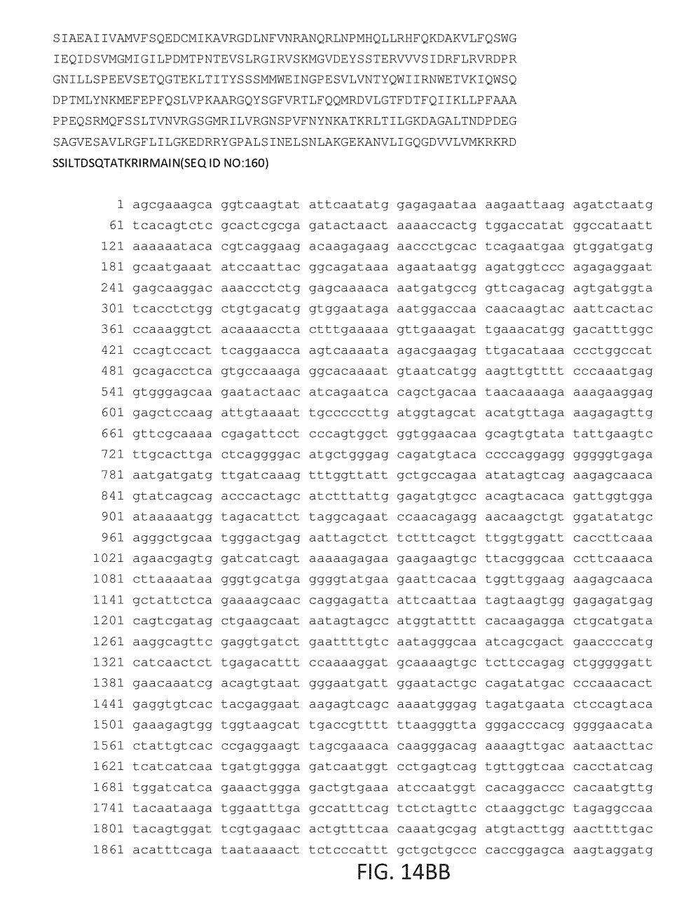

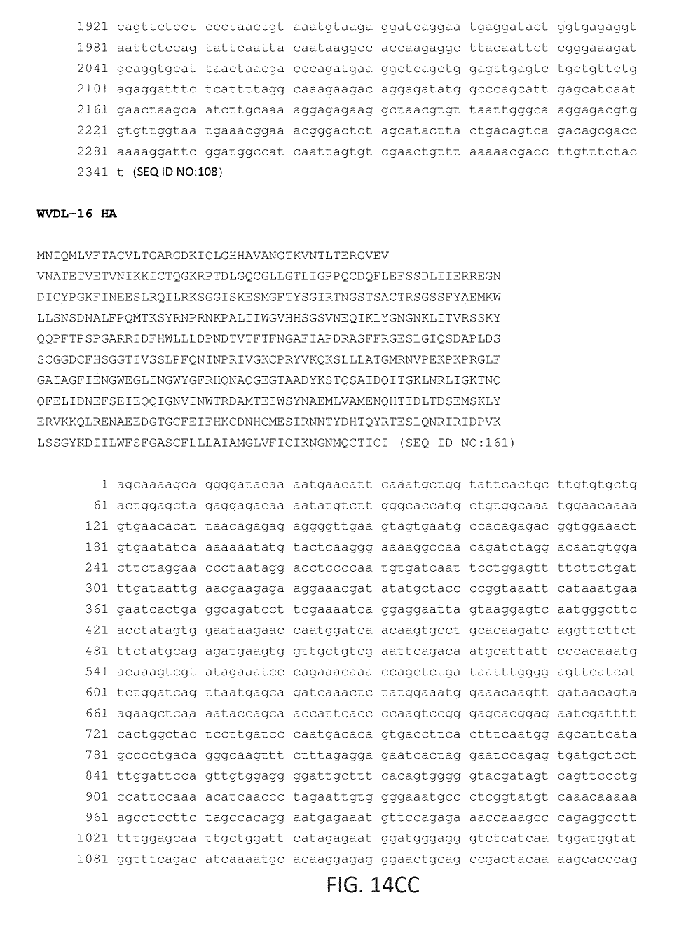

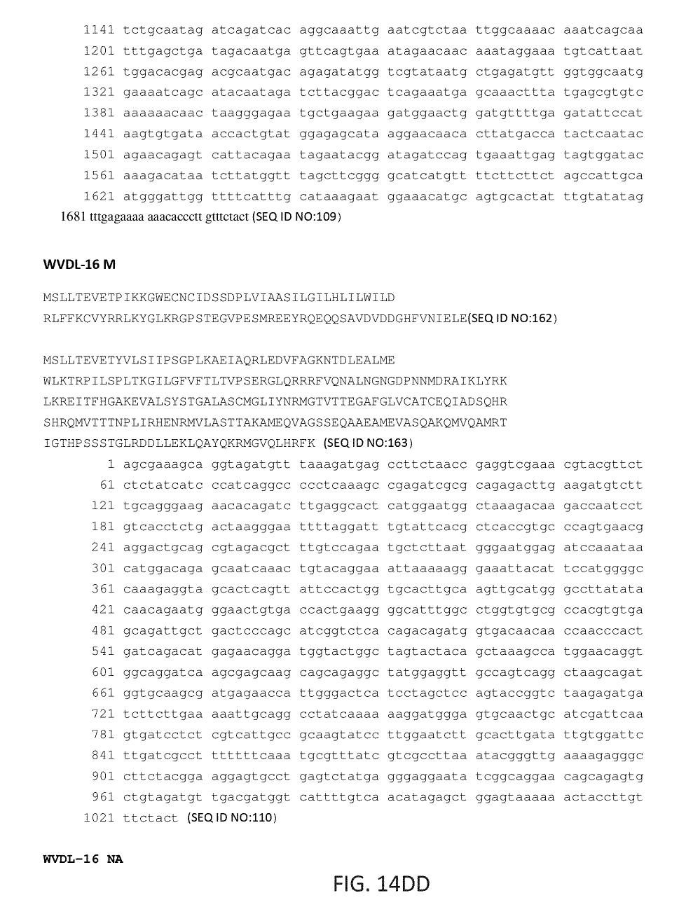

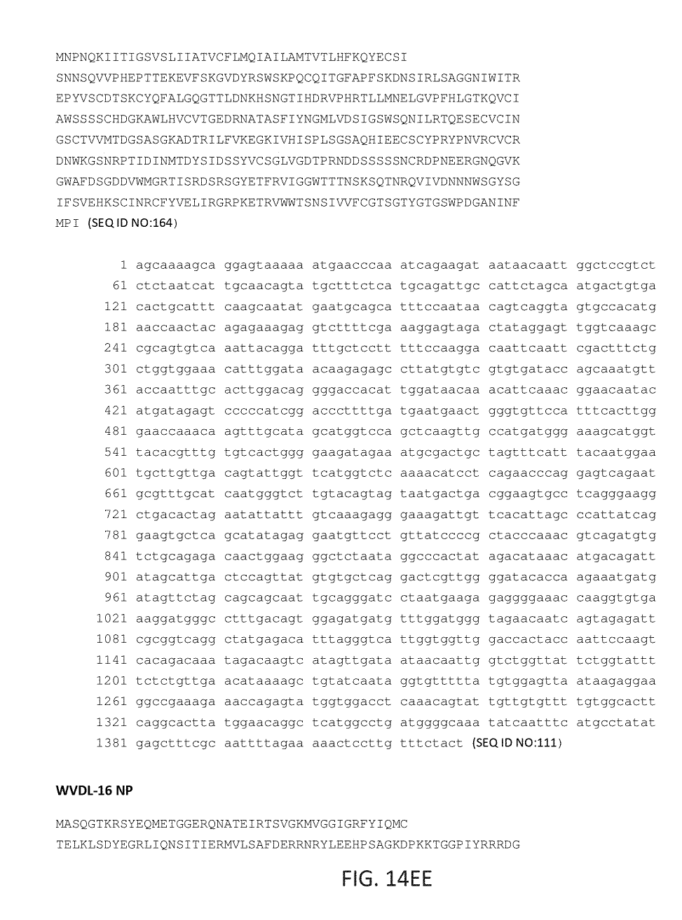

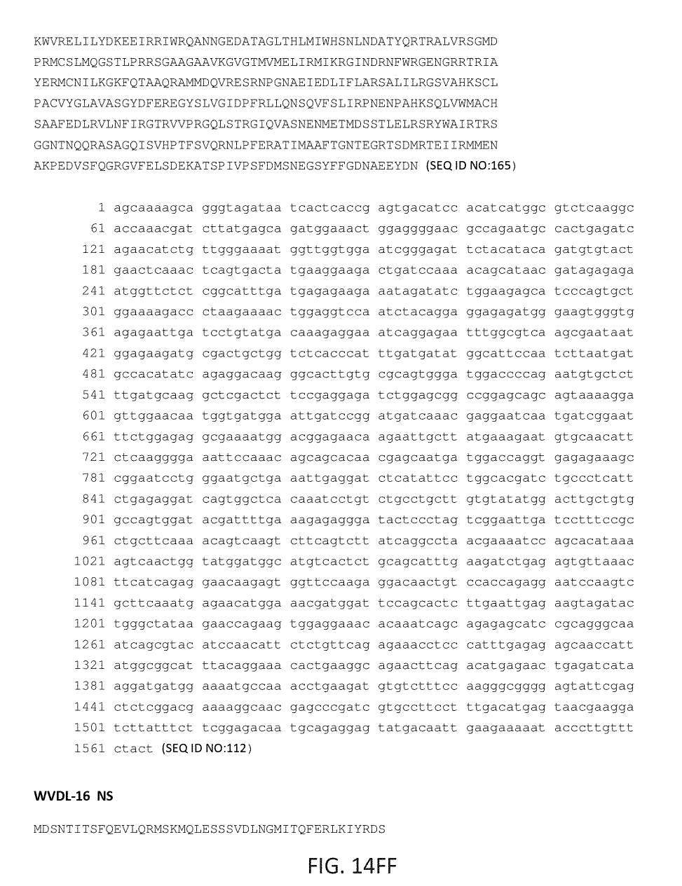

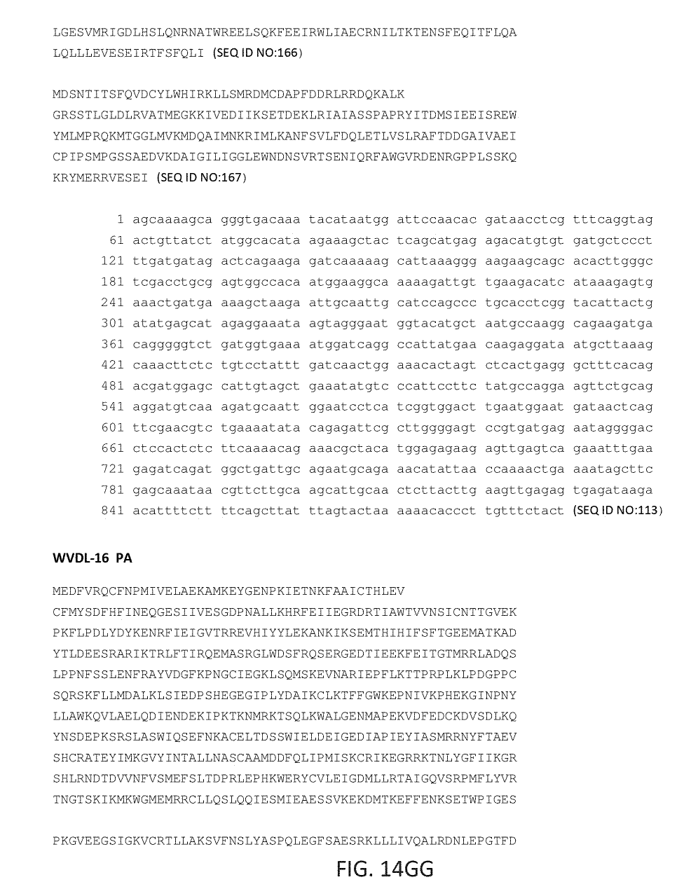

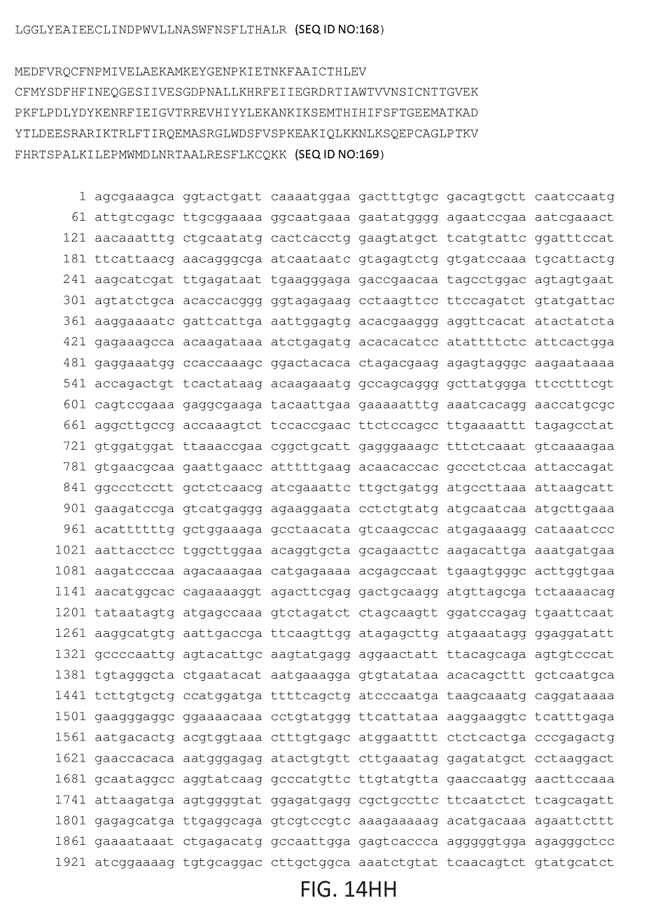

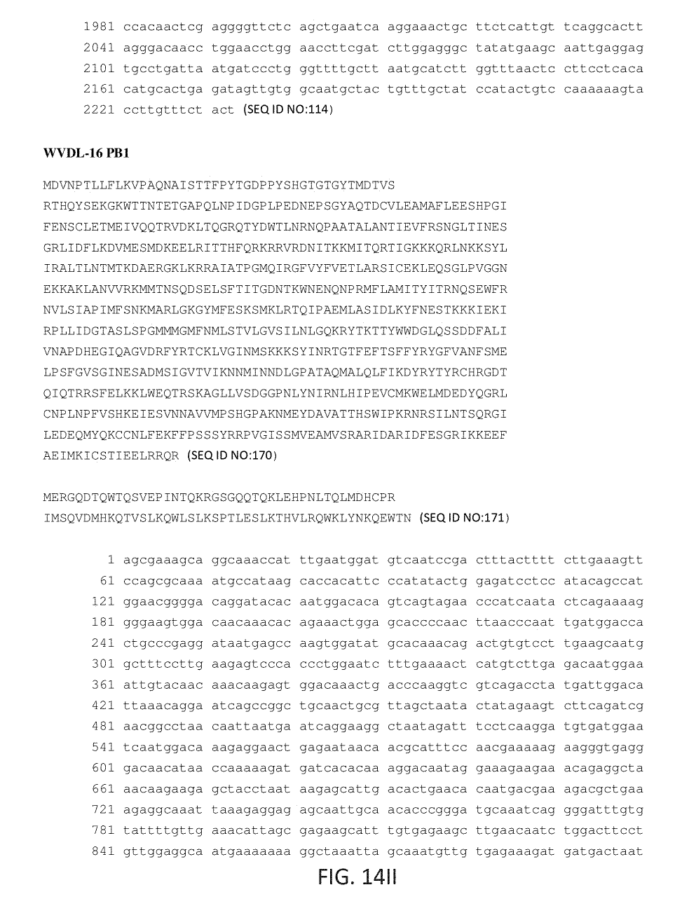

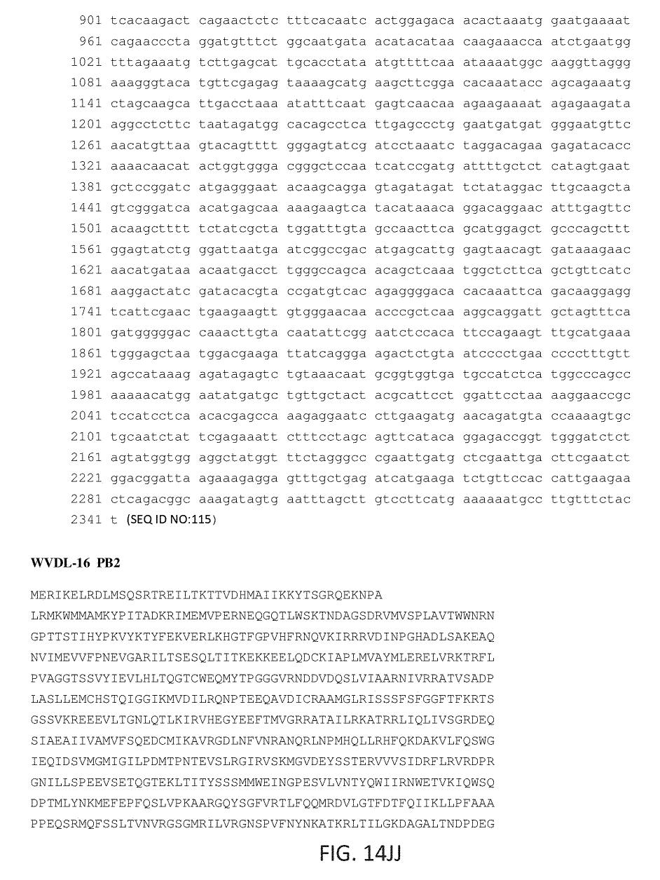

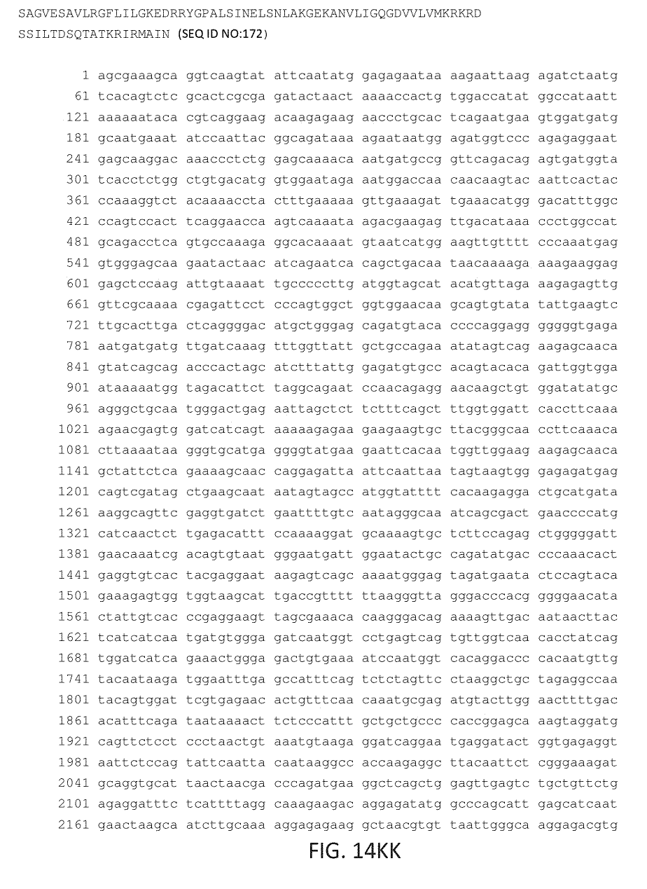

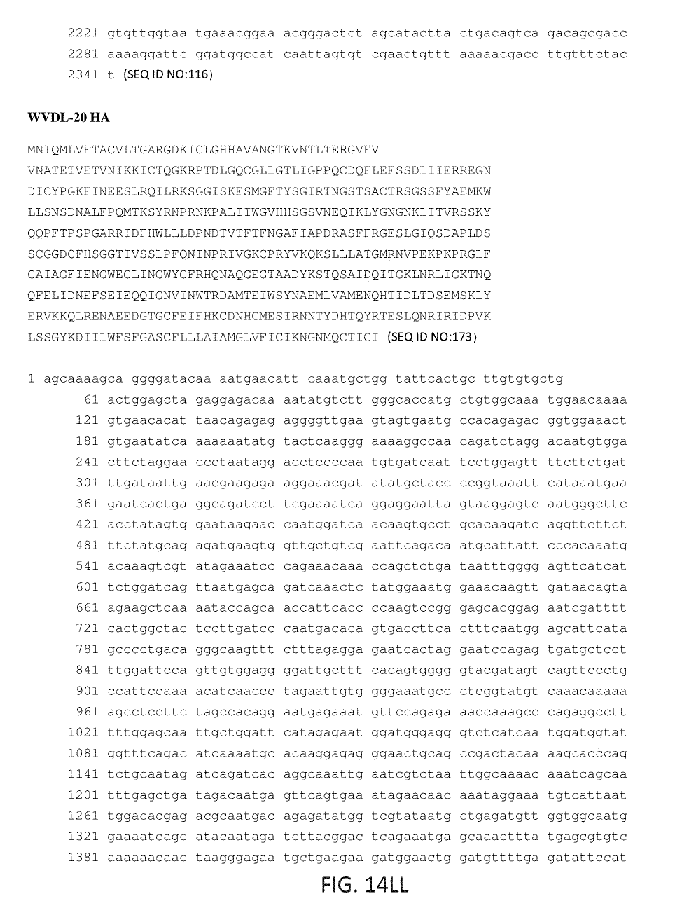

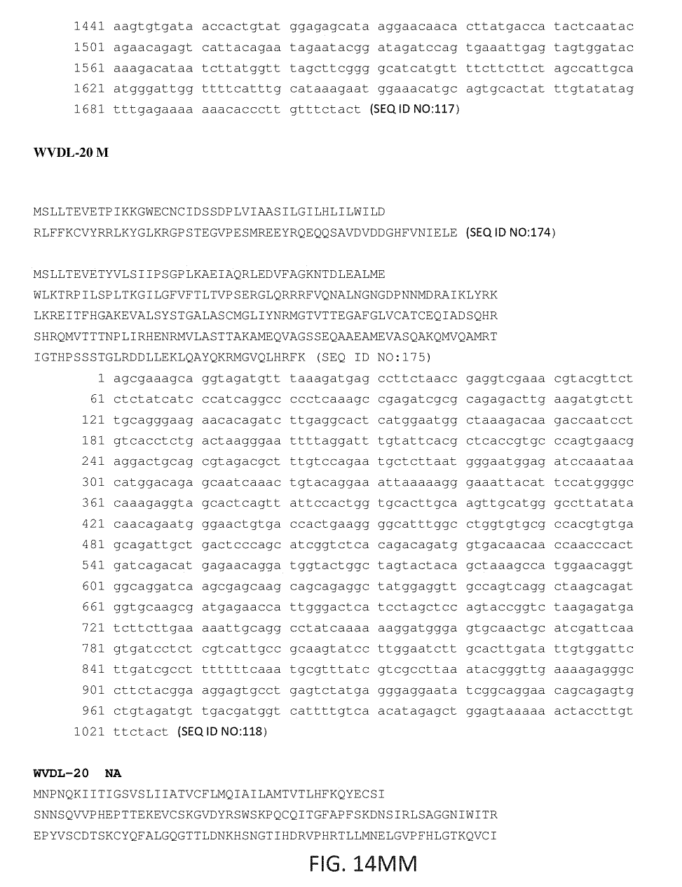

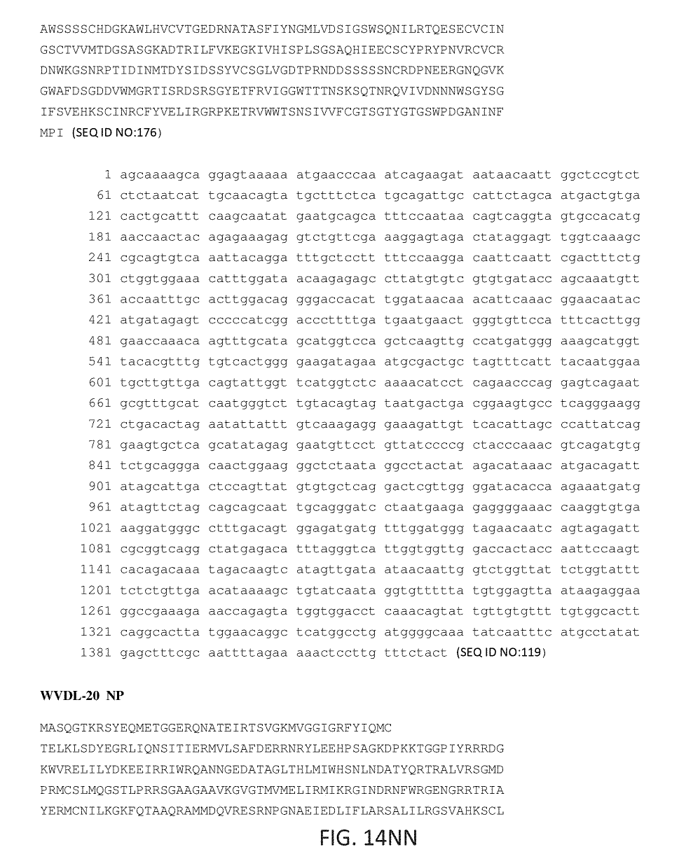

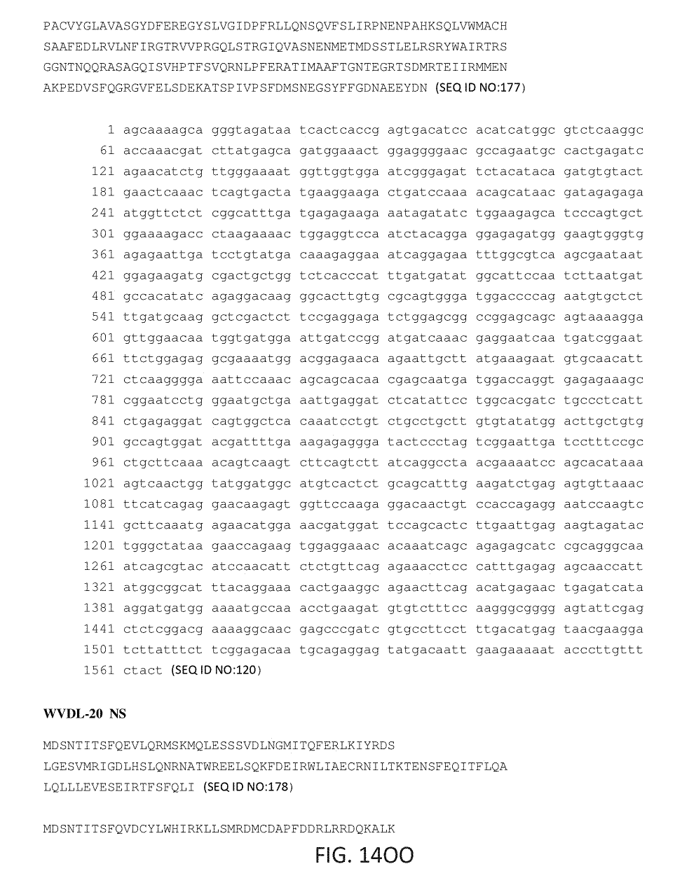

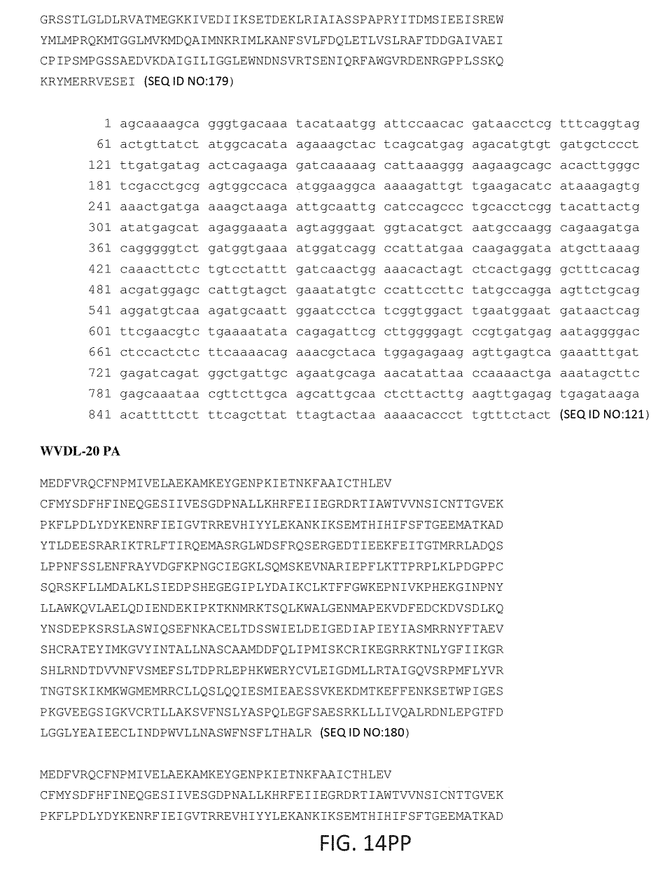

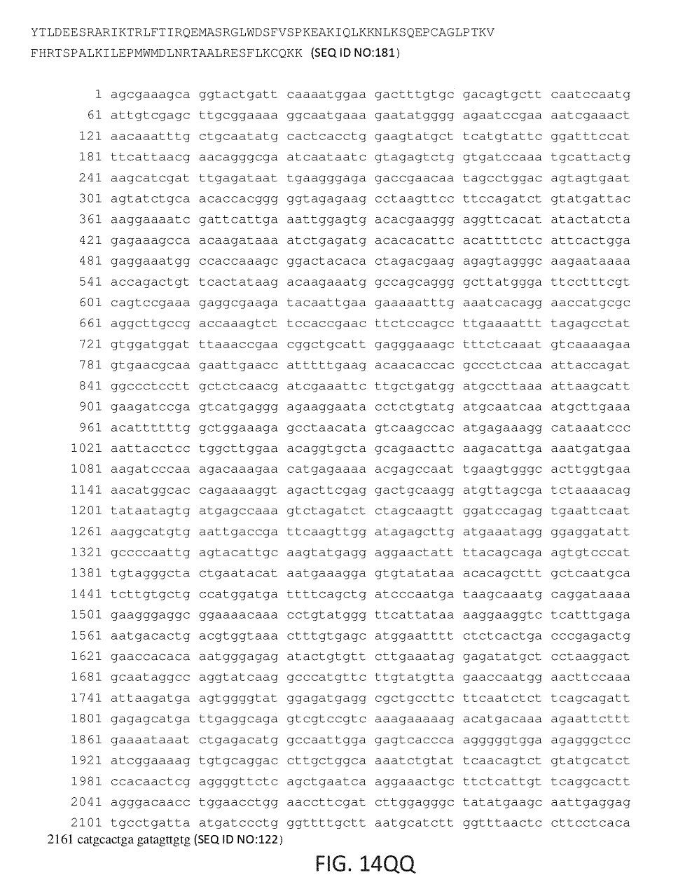

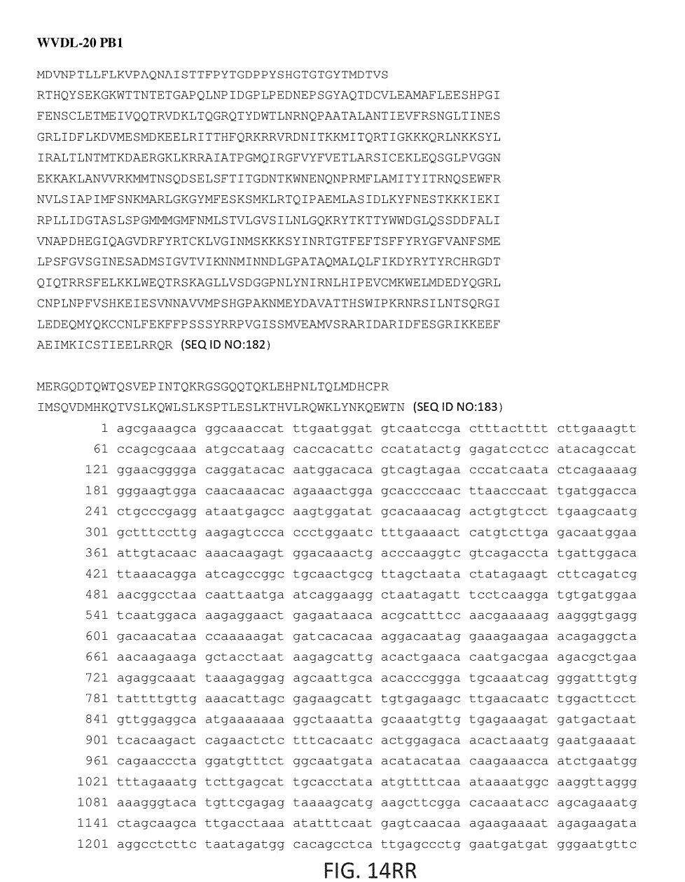

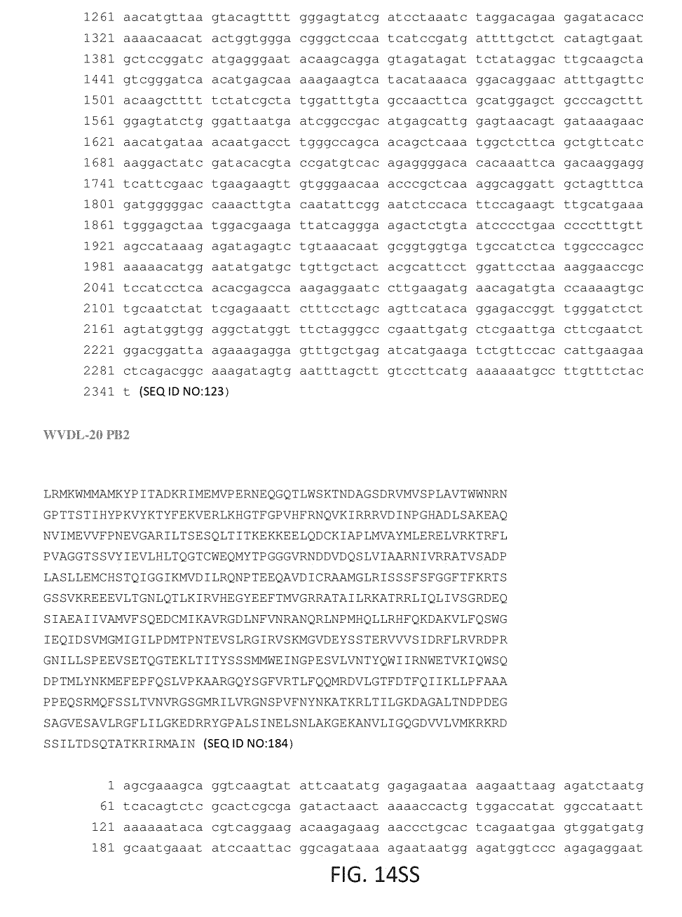

Ferrets intranasally infected with 10⁶ PFU of A/feline/NY/16 or A/chicken/NY/99 virus did not lose bodyweight (online Technical Appendix FIG. 13) but 2 of the ferrets infected with A/chicken/NY/99 virus had high fevers on day 1 postinfection. Both viruses replicated efficiently in the nasal turbinates and were also isolated from the trachea and lungs of some animals (Table 2), consistent with similar antigen distributions for both viruses (online Technical Appendix FIG. 14). No viruses were isolated from any of the other organs tested.

### Replication and Pathogenicity of Feline and Avian H7N2 Subtype Viruses in Cats

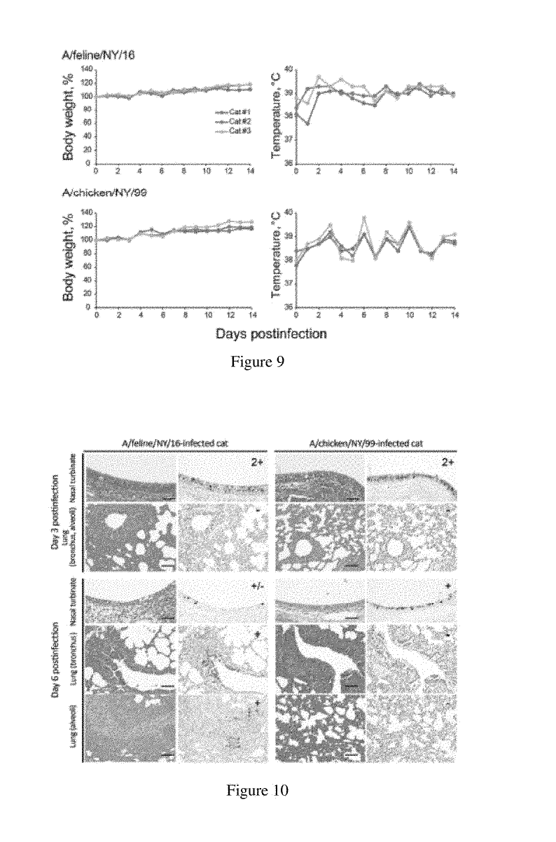

The infection of about 500 cats with H7N2 subtype viruses in animal shelters in New York in December 2016 suggested efficient replication of these viruses in felines. However, it was unclear whether these viruses were restricted to the respiratory organs or caused systemic infection. Cats intranasally infected with 10⁶ PFU of A/feline/NY/16 or A/chicken/NY/99 did not lose bodyweight (FIG. 9); however, fever was detected in 1 animal infected with A/feline/NY/16, and 1 infected with A/chicken/NY/99 virus; and a different animal infected with A/feline/NY/16 sneezed intensely on day 3 postinfection, but recovered fully.

A/feline/NY/16 virus replicated efficiently in the nasal turbinates, trachea, and lungs of infected cats (with the exception of 1 cat with a virus-negative lung sample on day 3 postinfection; Table 2). We isolated A/chicken/NY/99 virus mostly from nasal turbinates, with limited replication in the trachea and lung. These findings are consistent with the detection of A/feline/NY/16 antigen in both the upper and lower respiratory organs of infected cats, whereas A/chicken/NY/99 antigen was detected mainly in the nasal turbinates (FIG. 10). A/feline/NY/16 and A/chicken/NY 799 viruses were also isolated from the jejunum or colon of some of the infected animals (Table 2), although viral antigen was not detected in the intestines of cats infected with A/chicken/NY/99 or A/feline/NY/16 virus. These results demonstrate that the feline H7N2 virus replicates efficiently in the upper and lower respiratory tract of cats, reflecting adaptation of the virus to its new host.

TABLE 2
Virus titers in organs of ferrets and cals infected with A/feline/NY/16 or A/chicken/NY/99 influenza
A(H7N2) viruses. New York, NY, USA*

| | | | Virus titers in organs of infected | | | | | |
|---|---|---|---|---|---|---|---|---|
| | | | animals, log₁₀ PFU/g | | | | | |
| | Days | Animal ID | Nasal | | | Small | | Other |
| Species and virus | postinfection | no. | turbinates | Trachea | Lung | Intestine | Colon | organs† |
| Ferret | | | | | | | | |
| A/feline/NY/16 | 3 | 1 | 4.4 | 3.3 | 4.7 | — | — | — |
| | | 2 | 5.2 | — | 2.4 | — | — | — |
| | | 3 | 5.4 | — | — | — | — | — |
| | 6 | 4 | 3.1 | — | — | — | — | — |
| | | 5 | 5.8 | — | — | — | — | — |
| | | 6 | 6.0 | — | — | — | — | — |
| A/chicken/NY/99 | 3 | 7 | 5.9 | 3.3 | — | — | — | — |
| | | 8 | 6.0 | — | — | — | — | — |
| | | 9 | 6.6 | 3.4 | — | — | — | — |
| | 6 | 10 | 4.2 | — | — | — | — | — |
| | | 11 | 4.5 | — | 5.7 | — | — | — |
| | | 12 | 4.4 | — | — | — | — | — |
| Cat | | | | | | | | |
| A/feline/NY/16 | 3 | 1 | 3.9 | 4.8 | — | — | — | — |
| | | 2 | 4.1 | 6.6 | 5.8 | — | — | — |
| | | 3 | 6.9 | 7.0 | 5.8 | — | — | — |
| | 6 | 4 | 6.3 | 6.2 | 6.1 | — | 2.3 | — |
| | | 5 | 7.7 | 7.8 | 4.7 | — | — | — |
| | | 6 | 5.9 | 6.2 | 6.7 | 3.8 | — | — |
| A/chicken/NY/99 | 3 | 7 | 6.4 | 5.8 | 3.9 | — | — | — |
| | | 8 | 2.0 | — | — | — | — | — |
| | | 9 | 6.1 | — | — | 4.9 | — | — |
| | 6 | 10 | 6.4 | — | 5.0 | — | — | — |
| | | 11 | 4.6 | — | — | — | — | — |
| | | 12 | 6.7 | 4.0 | — | — | — | — |

*no virus detected.
†Brain, spleen, Kidneys, liver, and pancreas.

All cats infected with the A/feline/NY/16 virus exhibited histologic lesions in their nasal turbinates, tracheas, and lungs. Nasal turbinate pathology was moderate to severe in 5 of 6 cats with multifocal to diffuse distribution of lesions (FIG. 11, panel A). The tracheas of these cats exhibited mild to moderate histopathology (FIG. 11, panel B), whereas the lungs exhibited multifocal to coalescing histopathology centered mostly on the bronchioles, with 3 of 6 cats possessing moderately severe lesions (FIG. 11, panel C). Similar histopathological changes were found in cats infected with A/chicken/NY/99 virus. Appreciable histopathology was also noted in the small intestine (duodenum) of cats infected with A/feline/NY/16 and A/chicken/NY/99 viruses (FIG. 11, panel D; cat ID nos. 1, 2, 4, 8, 10, and 12 in Table 2), although virus was detected in the intestines of only 3 cats (cat ID nos. 4, 6, and 9 in Table 2). The correlation between virus replication and histologic lesions in cat intestines is currently unknown.

### Transmission of Feline and Avian H7N2 Subtype Viruses in Ferrets and Cats

The fulminant spread of the feline H7N2 subtype viruses among cats, and the confirmed H7N2 virus infection of a veterinarian who treated the animals, indicate that these originally avian influenza viruses have the ability to transmit among mammals. To test the transmissibility of feline and avian H7N2 subtype viruses in ferrets, 3 animals per group (each placed in a separate cage) were infected intranasally with 10⁶ PFU (500 μL) of A/feline/NY/16 or A/chicken/NY/99 virus. One day later, 1 naive ferret was housed with each of the infected ferrets (direct contact transmission experiment), or placed naive ferrets in wireframe cages (within transmission isolators) ≈5 cm from the cages containing the infected ferrets as a respiratory droplet transmission experiment. Nasal wash samples from infected, contact, and exposed animals were collected on day 1 after infection, contact, or exposure, and then every other day; we determined virus titers in nasal wash samples by use of plaque assays in MDCK cells (Table 3). In respiratory droplet transmission experiments, ferrets infected with A/feline/NY/16 or A/chicken/NY/99 virus secreted virus, but exposed animals were virus negative and did not seroconvert (Table 3). Among the direct contact animals, we detected virus in 1 ferret from the A/feline/NY/16-inoculated group and 2 from the A/chicken/NY/99-inoculated group; these 3 animals seroconverted, although the HI titer of 1 of the animals was low,

TABLE 3
Influenza A(H7N2) virus titers in nasal wash samples from ferret transmission studies,
New York, NY, USA*

| | | | Virus titers in nasal | | | | | | |
|---|---|---|---|---|---|---|---|---|---|
| | | | wash samples by days | | | | | | |
| | | | after infection, | | | | | | |
| | | | exposure, or contact, | | | | | | |
| Virus and transmission | | | log₁₀ PFU/mL | | | | | | Seroconversion, |
| mode | Pair | Action | 1 | 3 | 5 | 7 | 9 | 11 | HI titer† |
| A/feline/NY16 | | | | | | | | | |
| Respiratory droplets | 1 | Infection | 4.2 | 5.6 | 5.0 | — | — | — | 320 |
| | | Exposure | — | — | — | — | — | — | <10 |
| | 2 | Infection | 4.6 | 4.3 | 5.4 | — | — | — | 320 |
| | | Exposure | — | — | — | — | — | — | <10 |
| | 3 | Infection | 5.3 | 5.0 | 4.8 | 2.8 | — | — | 640 |
| | | Exposure | — | — | — | — | — | — | <10 |
| Direct contact | 1 | Infection | 3.6 | 4.1 | 5.0 | 2.0 | — | — | 640 |
| | | Contact | — | — | — | — | — | — | <10 |
| | 2 | Infection | 5.5 | 5.1 | 4.3 | 1.3 | — | — | 320 |
| | | Contact | — | — | — | — | — | — | <10 |
| | 3 | Infection | 5.0 | 5.2 | 5.2 | 2.9 | — | — | 640 |
| | | Contact | — | — | 4.2 | 5.3 | 4.6 | — | 320 |
| A/chicken/NY/99 | | | | | | | | | |
| Respiratory droplets | 1 | Infection | 5.8 | 4.0 | 4.3 | — | — | — | 160 |
| | | Exposure | — | — | — | — | — | — | <10 |
| | 2 | Infection | 5.6 | 4.2 | 3.5 | — | — | — | 160 |
| | | Exposure | — | — | — | — | — | — | <10 |
| | 3 | Infection | 5.1 | 3.7 | 3.5 | — | — | — | 320 |
| | | Exposure | — | — | — | — | — | — | <10 |
| Direct contact | 1 | Infection | 4.3 | 4.3 | 3.0 | — | — | — | 160 |
| | | Contact | — | — | 3.8 | 4.3 | 3.4 | — | 160 |
| | 2 | Infection | 4.2 | 3.8 | 3.8 | — | — | — | 160 |
| | | Contact | — | — | 2.1 | — | — | — | 10 |
| | 3 | Infection | 4.9 | 3.9 | 4.3 | — | — | — | 320 |
| | | Contact | — | — | — | — | — | — | 10 |

*HI, hemagglutination inhibition;
—, no virus detected.
†Serum specimens were collected on day 18 after infection, exposure, or contact, and examined using an HI assay. The HI fiter is the inverse of the highest dilution of serum that completely inhibited hemagglutination.

The transmission study in cats was conducted in the same way as the study in ferrets; cages were spaced 35 cm apart to prevent direct contact between the inoculated and exposed animals (online Technical Appendix FIG. 1, panel A). All infected cats secreted viruses for 5-7 days after infection and seroconverted, except for 1 cat infected with A/chicken/NY/99 virus, which seroconverted but did not shed virus (Table 4). A/chicken/NY/99 virus was not isolated from contact or exposed animals, although these animals seroconverted (Table 4), Direct contact transmission of A/feline/NY/16 virus was detected in all 3 pairs of cats, with both seroconversion and virus isolation in 2 pairs (Table 4). Respiratory droplet transmission of A/feline/NY/16 occurred in 2 pairs of animals, with high virus titers detected in the nasal secretions of the exposed animals on days 9 and 11 postexposure, respectively; both of the exposed animals also seroconverted (Table 4), in the third transmission pair, the exposed animal did not shed virus or seroconvert. Taken together, we demonstrated that A/feline/NY/16 virus has the ability to transmit among cats via contact and respiratory droplets; the relative contribution of these modes of transmission to the H7N2 subtype virus outbreaks in cat shelters in New York is unknown.

TABLE 4
Influenza A(H7N2) virus titers in nasal swab samples from cat transmission studies,
New York, NY, USA*

| | | | Virus titers in nasal | | | | | | | |
|---|---|---|---|---|---|---|---|---|---|---|
| | | | swab samples by days | | | | | | | |
| | | | after infection, | | | | | | | |
| | | | exposure, or contact, | | | | | | | |
| Virus and transmission | | | log₁₀ PFU/mL | | | | | | | Seroconversion, |
| mode | Pair | Action | 1 | 3 | 5 | 7 | 9 | 11 | 13 | HI titer† |
| A/feline/NY/16 | | | | | | | | | | |
| Respiratory droplets | 1 | Infection | 5.6 | 4.7 | 4.3 | 3.0 | — | — | — | 320 |
| | | Exposure | — | — | — | — | 5.2 | — | — | 160 |
| | 2 | Infection | 4.5 | 2.7 | 5.0 | 4.6 | — | — | — | 320 |
| | | Exposure | — | — | — | — | — | 5.4 | — | 80 |
| | 3 | Infection | 4.8 | 3.2 | 5.3 | 3.6 | — | — | — | 320 |
| | | Exposure | — | — | — | — | — | — | — | <10 |
| Direct contact | 1 | Infection | 5.9 | 4.6 | 3.4 | — | — | — | — | 320 |
| | | Contact | — | — | 2.0 | 5.4 | — | — | — | 320 |
| | 2 | Infection | 6.0 | 5.0 | 4.6 | — | — | — | — | 640 |
| | | Contact | — | — | — | — | — | — | — | 80 |
| | 3 | Infection | 4.9 | 4.9 | 4.4 | 4.2 | — | — | — | 640 |
| | | Contact | — | 5.2 | 5.4 | 5.2 | — | — | — | 160 |
| A/chicken/NY/99 | | | | | | | | | | |
| Respiratory droplets | 1 | Infection | 4.0 | 3.7 | 4.7 | — | — | — | — | 320 |
| | | Exposure | — | — | — | — | — | — | — | 20 |
| | 2 | Infection | — | — | — | — | — | — | — | 320 |
| | | Exposure | — | — | — | — | — | — | — | 20 |
| | 3 | Infection | 2.6 | 1.6 | 2.3 | — | — | — | — | 80 |
| | | Exposure | — | — | — | — | — | — | — | 160 |
| Direct contact | 1 | Infection | 4.5 | 2.4 | 4.5 | — | — | — | — | 80 |
| | | Contact | — | — | — | — | — | — | — | 160 |
| | 2 | Infection | 3.4 | 4.8 | 4.1 | 3.6 | — | — | — | 160 |
| | | Contact | — | — | — | — | — | — | — | 40 |
| | 3 | Infection | 3.4 | 3.5 | 3.2 | 3.3 | — | — | — | 160 |
| | | Contact | — | — | — | — | — | — | — | 20 |

*HI, hemagglutination inhibition;
—, no virus detected.
†Serum samples were collected on day 18 after infection, exposure, or contact, and examined by use of an HI assay. The HI fiter is the inverse of the highest dilution of serum that completely inhibited hemagglutination.

### Receptor-Binding Specificity of Feline and Avian H7N2 Subtype Viruses

Avian influenza viruses isolated from their natural reservoir (i.e., wild aquatic birds) are often restricted in their ability to infect mammalian cells because of their preferential binding to α2,3-linked sialic acids, whereas most human influenza viruses preferentially bind to α2,6-linked sialic acids (Connor et at, 1994; Gambaryan et al., 1997; Matrosovich et al., 1997). Glycan array analysis was performed with A/feline/NY/16, A/chicken/NY/99, and Kawasaki/173-PR8, a control virus possessing the HA and NA genes of the seasonal human A/Kawasaki/173/2001 (H1N1) virus and the remaining genes from A/PR/8/34 (H1N1) virus. As expected, Kawasaki/173-PR8 virus bound to α2,6-linked sialosides (FIG. 12; online Technical Appendix Table 2). A/chicken/NY/99 virus bound to both α2,6- and α2,3-linked sialosides, consistent with the dual avian/human receptor-binding specificity of influenza viruses isolated from land-based poultry (Wright et al. 2013). Of note, A/feline/NY/16 virus bound strongly to α2,3-linked sialosides (i.e., avian-type receptors) with negligible binding to human-type receptors.

Next, the prevalence of α2,3- and α2,6-linked sialosides in the feline airway and intestines of an immunologically naive cat was examined by using lectins that detect α2,3-linked (i.e., MAA I and MAA II) and α2,6-linked sialosides (i.e., SNAI). MAA I and MAIA II bound to epithelial cells throughout the feline airway, whereas SNA binding was detected only in the trachea and bronchus (FIG. 13), consistent with the findings of other research groups (Wang et al., 2013; Said et al., 2011; Thongratsakul et at, 2010). Sialosides were not detected in the cat intestine. The predominance of avian-type receptors in the upper respiratory tract of felines may have led to the selection of feline H7N2 virus HA proteins with preferential binding to α2,3-linked sialosides.

### Sensitivity to Neuraminidase Inhibitors

To test whether infections with the feline H7N2 viruses could be treated with neuraminidase (NA) inhibitors, the sensitivity of A/feline/NY/16 and A/chicken/NY/99 to several-NA inhibitors (i.e., oseltamivir, zanamivir, and laninamivir) was assessed by determining the 50% inhibitory concentration (IC₅₀) of the NA enzymatic activity. A/Anhui/1/2013 (H7N9) virus was used as an NA inhibitor-sensitive control and its NA inhibitor-resistant variant, A/Anhui/1/2013-NA-R294K, as an NA inhibitor-resistant control (online Technical Appendix Table 3). A/feline/NY/16 and A/chicken/NY/99 were sensitive to all of the NA inhibitors tested (Technical Appendix Table 3), consistent with the absence of amino acid residues in the NA protein that are known to confer resistance to NA inhibitors. Hence, NA inhibitors could be used to treat persons infected with feline H7N2 subtype viruses.

### Discussion

In the present study, it was demonstrated that a feline H7N2 subtype virus isolated during an outbreak in an animal shelter in New York in December 2016 replicated well in the respiratory organs of mice and ferrets but did not cause severe symptoms. The efficient replication of the feline H7N2 subtype viruses in the respiratory organs of several mammals, combined with the ability of these viruses to transmit among cats (albeit inefficiently) and to infect 1 person, suggest that these viruses could pose a risk to human health. Close contacts between humans and their pets could lead to the transmission of the feline viruses to humans. To protect public health, shelter animals (where stress and limited space may facilitate virus spread) should be monitored closely for potential outbreaks of influenza viruses.

The present findings of mild disease in mice and ferrets are consistent with the recent report by Belser et al. (2017) who studied the H7N2 subtype virus isolated from an infected veterinarian, Feline H7N2 virulence in cats was assessed and efficient virus replication was detected in both the upper and lower respiratory organs of infected animals, whereas an avian H7N2 subtype virus was detected mainly in the nasal turbinates.

Belser et al. (2017) reported that intranasal or aerosol infection of ferrets with the H7N2 virus isolated from the infected veterinarian did not result in the seroconversion of co-housed or exposed animals, although nasal wash samples from some of the co-housed ferrets contained low titers of virus; these findings may, suggest limited virus transmission that was insufficient to establish a productive infection. In contrast, we detected feline H7N2 virus transmission to co-housed ferrets in 1 of 3 pairs tested; this difference may be explained by the amino acid differences in the PA, HA, and NA proteins of the feline and human H7N2 isolates (online Technical Appendix Table 4) or by the small number of animals used in these studies. Transmission studies were performed in cats and detected feline H7N2 subtype virus transmission via direct contact and respiratory droplets. However, the group size used is a potential limitation of our study.

Cats are not a major reservoir of influenza A viruses, but can be infected naturally or experimentally with influenza viruses of different subtypes (Harder et al., 2009). Serologic surveys suggest high and low rates of seroconversion to seasonal human and highly pathogenic avian influenza viruses, respectively. Natural infections most likely result from close contact with infected humans or animals, and most of these infections appear to be self-limiting.

Few cases of human infections with influenza viruses of the H7 subtype were reported until 2013, and they typically caused mild illness; however, infection of a veterinarian with a highly pathogenic avian H7N7 virus had fatal consequences (Fouchier et al., 2004; Koopmans et al., 2004). Since 2013, influenza viruses of the H7N9 subtype have caused more than 1,300 laboratory-confirmed infections in humans, with a case-fatality rate of ≈30%. Although the current H7N9 and feline H7N2 subtype viruses do not exclusively bind to human-type receptors and do not transmit efficiently among humans, the spread and biologic properties of these viruses should be monitored carefully.

### Example 4

Avian influenza viruses occasionally cross the species barrier, infecting humans and other mammals after exposure to infected birds and contaminated environments. Unique among the avian influenza A subtypes, both low pathogencity and highly pathogenic H7 viruses have demonstrated the ability to infect and cause disease in humans (Belser et al., 2009; World Health Org., 2016). In the eastern and northeastern United States, low pathogenicity avian influenza (LPA) A(H7N2) viruses circulated in live bird markets periodically during 1994-2006 (Senne et al., 2003(a)) and caused poultry outbreaks in Virginia, West Virginia, and North Carolina in 2002 (Senne 2003(b)). During an outbreak in Virginia in 2002, human infection with H7N2 virus was serologically confirmed in a culler with respiratory, symptoms (CDC, 2004). In 2003, another human case of H7N2 infection was reported in a New York resident (Ostrowsky et al., 2003); although the source of exposure remains unknown, the isolated virus was closely related to viruses detected in live bird markets in the region. Because of the sporadic nature of these and other zoonotic infections with influenza H7 viruses throughout the world, the World Health Organization (WHO) recommended development of several candidate vaccine viruses for pandemic preparedness purposes, including 2 vaccines derived from North American lineage LPAI viruses, A/turkey/Virginia/4529/2002 and A/New York/107/2003 (Pappas et al., 2007).

An outbreak of influenza A (H7N2) virus in cats in a shelter in New York, N.Y., USA, resulted in zoonotic transmission. Virus isolated from the infected human was closely related to virus isolated from a cat; both were related to low pathogenicity avian influenza A (H7N2) viruses detected in the United States during the early 2000s.

The Study

On Dec. 19, 2016, the New York City Department of Health and Mental Hygiene collected a respiratory specimen from a veterinarian experiencing influenza-like illness after exposure to sick domestic cats at an animal shelter in New York, N.Y., USA. The specimen tested positive for influenza A but could not be subtyped. Specimen aliquots were shipped to the Wadsworth Center, New York State Department of Health (Albany, N.Y., USA), and to the Centers for Disease Control and Prevention (CDC; Atlanta, Ga., USA). Next-generation sequencing performed at the New York State Department of Health generated a partial genomic sequence (6 of 8 influenza A virus gene segments) that aligned most closely with North American lineage LPAI A (H7N2) viruses. North American lineage H7 real-time reverse transcription PCR (rRT-PCR) testing and diagnostic sequence analysis performed at CDC confirmed the sample to be positive for influenza A (H7N2) virus. Virus isolation was attempted by inoculating the sample in 10-day-old embryonated chicken eggs and MDCK CCL-34 and CRFK (Crandell-Rees Feline Kidney) cell lines (American Type Culture Collection). A/New York/108/2016 was successfully isolated from eggs but not from MDCK or CRFK cells. Codon complete sequencing of the egg-isolated virus (GISAID accession nos. EPI944622-9; http://www.gisaid.org) showed no nucleotide changes compared with the hemagglutinin (HA) and neuraminidase (NA) gene segments sequenced directly from the clinical specimen. The virus was nearly identical (99.9%) to a virus isolated from a cat, A/feline/New York/16-040082-1/2016, from a New York shelter where the veterinarian had worked; the cat died of its illness. Phylogenetic analysis of the cat and human viruses showed that their genomes were closely related to LPAI A(H7N2) viruses that were circulating in the northeastern United States in the early 2000s (online Technical Appendix Figure, https://wwwnc.cdc.gov/EID/article/23/12/17-0798-Techapp1.pdf).

Analysis of the HA gene segments revealed that A/New York/108/2016 and A/feline/New York/16-040082-1/2016 were phylogenetically related to H7N2 viruses isolated from poultry in the eastern United States (New York, Virginia, Pennsylvania, North Carolina, Massachusetts) during 1996-2005, including 2 influenza A(H7N2) WHO-recommended candidate vaccine viruses. Although the internal protein coding gene segments (polybasic 1 and 2, polyacidic, nucleoprotein, matrix, nonstructural) were distant to sequences available in databases (average nucleotide identity to the closest genetic relative was 97.6%), analysis indicated that they were of LPAI virus origin and lacked known mammalian adaptive substitutions. The longer branch lengths of the internal protein coding gene segments highlighted the scarcity of sequence data available for contemporary H7N2 viruses in the United States.

Similar to well-characterized H7N2 viruses, such as A/turkey/Virginia/4529/2002 and A/New York/107/2003, A/New York/108/2016 had deletion of amino acids 212-219 in the mature HA protein (H7 numbering), known as the 220-loop of the HA receptor binding domain (Yang et al., 2010). Such deletion has been previously shown to enhance binding and infectivity of H7 viruses to the mammalian respiratory tract and increase direct contact transmission between mammals (Belser et al., 2009). Glycan microarray analysis showed that A/New York/108/2016 bound preferentially to α-2,3 avian-like receptors but also showed binding to the α-2,6 glycan with internal sialoside (LSTb, glycan #60), as well as to glycans with mixed α-2,3/α-2,6 receptors (FIG. 14). Strong binding to the LSTb glycan has been previously reported for North America H7N2 viruses of avian origin (Yang et al., 2010; Belser et al., 2008) and 2013 human H7N9 viruses (Yang et al., 2013). The role of the LSTb glycan binding remains unknown; it has been identified only in human milk (Weinstein et al., 1982).

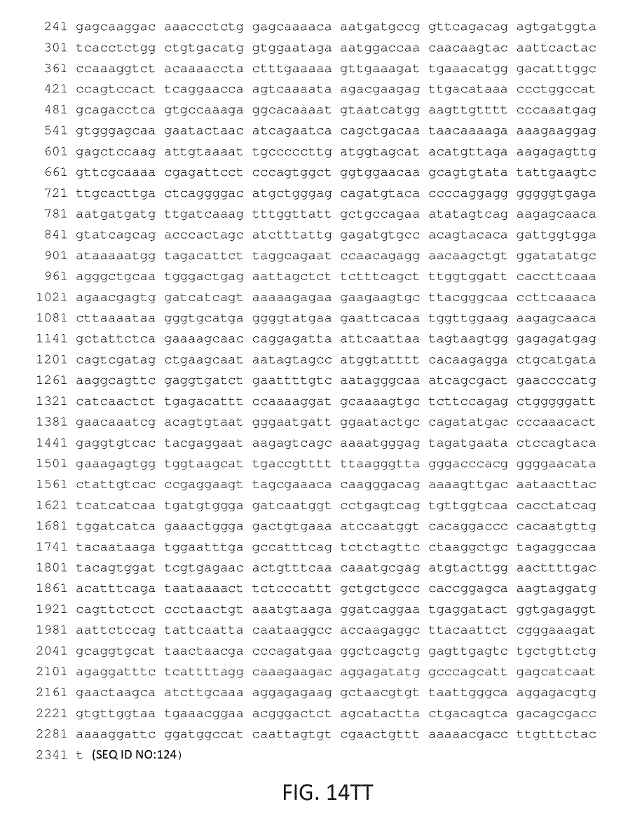

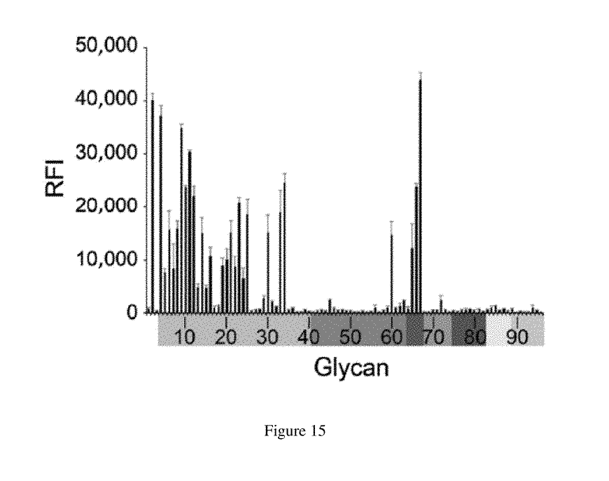

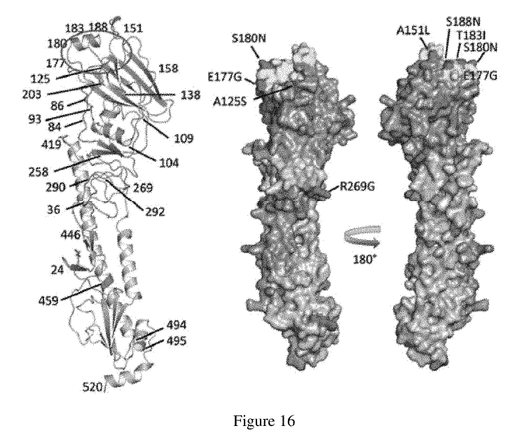

Additional molecular characterization of the HA1 protein showed 20 aa differences between A/New York/108/2016 and A/turkey/Virginia/4529/2002 (26 aa in both HA1 and HA2; FIG. 15). The substitution A1255 resulted in a gain of glycosylation in the HA protein of A/New York/108/2016, previously correlated with increased replication efficiency and wider tissue distribution of A/Netherlands/219/2003 (H7N7) (Fouchier et al., 2004). The substitution of T183I was shown in other avian influenza viruses (e.g., H5N1) to enhance binding to mammalian sialic acid receptors (Yang et al., 2007). Four of the 20 aa changes were in residues associated with antibody recognition at antigenic site B (E177G, S180N, T183I, and S188N) and antigenic site C (R269G).

TABLE 5
Table. Hemagglutination inhibition testing of influenza A(H7) virus isolated from cat
and human in New York, NY, USA, 2016, and reference viruses

| | Ferret antisera | | | | | | |
|---|---|---|---|---|---|---|---|
| | | α— | α— | α— | α— | | Normal ferret |
| Antigens | Subtype | Gs/NE | Tk/MN | Tk/VA | NY/107 | α-NY/108 | serum |
| Reference | | | | | | | |
| A/goose/Nebraska/17097-4/11 | H7/N9 | 160* | 80 | 160 | 80 | <10 | <10 |
| A/turkey/Minnesota/0141354/09 | H7/N9 | 20 | 80* | 20 | 20 | <10 | <10 |
| A/turkey/Virginia/4529/02 | H7/N2 | 40 | 10 | 160* | 640 | 10 | <10 |
| A/New York/107/03 | H7/N2 | 40 | 20 | 160 | 640* | 10 | <10 |
| A/New York/108/16† | H7/N2 | 40 | 10 | 80 | 80 | 320* | <10 |
| Test | | | | | | | |
| A/feline/New York/16-040082- | H7/N2 | 40 | 10 | 80 | 80 | 320 | <10 |
| 1/16 | | | | | | | |

*Indicates homologous titers.
α-, reference antiserum.
GS/NE, A/goose/Nebraska/17097/-4/11;
Tk/MN, A/turkey/Minnesota/0141354/09;
Tk/VA, A/turkey/Virginia/4529/02;
NY/107, A/New York/107/03;
NY/108, A/New York/108/16.
†Virus isolated from human (veterinarian who experienced influenza-like illness after exposure to sick domestic cats at an animal shelter).

To determine the effect of these differences on antigenicity, we assessed the relationships in a 2-way hemagglutination inhibition assay, using a panel of ferret antisera raised to related H7 viruses (Table 5). The results showed that A/New York/108/2016 and A/feline/New York/16-040082-1/2016 reacted with α-A/turkey/Virginia/4529/2002 postinfection ferret antiserum (2-fold reduction of the hemagglutination inhibition titer compared with the A/turkey/Virginia/4529/2002 homologous titer) and α-A/New York/107/2003 antiserum (8-fold reduction compared with the A/New York/107/2003 homologous titer). These data suggest that the A/turkey/Virginia/4529/2002 candidate vaccine virus would provide cross protection if vaccination against the 2016 H7N2 viruses was needed. Both A/turkey/Virginia/4529/2002 and A/New York/107/2003, however, reacted poorly with the antiserum raised against A/New York/108/2016.

A 20-aa deletion in the NA stalk region, considered a genetic marker of poultry-adapted viruses (Matrosovich et al., 1999), was also identified in the human and feline H7N2 viruses. No genetic markers known to reduce susceptibility to the NA inhibitor class of antiviral drugs were identified in the NA gene. Results of the NA inhibition assay indicated that the H7N2 viruses were susceptible to 4 NA inhibitors: oseltamivir, zanamivir, peramivir, and laninamivir (data not shown).

### CONCLUSIONS

The circulation of an influenza A(H7N2) virus at the animal-human interface, especially among common companion animals such as domestic cats, is of public health concern. Moreover, from an epidemiologic perspective, it is essential to understand the current distribution of LPAI A(H7N2) viruses in both avian and feline hosts. The US Department of Agriculture and state departments of agriculture have conducted routine avian influenza surveillance in live bird markets; 132,000-212,000 tests for avian influenza were performed annually during 2007-2014 (Myers, 2015), but LPAI A(H7N2) viruses were not detected. The acquisition of many genetic changes throughout the genome of the human and cat H7N2 viruses we report, however, suggests onward evolution of the virus since it was last detected in poultry and wild birds. The human virus bound to α-2,6-linked sialic acid receptors, which are more common in mammals, yet retained α-2,3-linked sialic acid binding, indicating that it has dual receptor specificity; this information can be used in pandemic risk assessment of zoonotic viruses.

### REFERENCES

Arafa et al., Sci. Rep., 6:38388 (2016).
Avery's Drug Treatment: Principles and Practice of Clinical Pharmacology and Therapeutics, 3rd edition, ADIS Press, Ltd., Williams and Wilkins, Baltimore, Md. (1987).
Aymard-Henry et al., Virology: A Practical Approach, Oxford IRL Press, Oxford, 119-150 (1985).
Bachmeyer, Intervirology. 5:260 (1975).
Belser et al., Emerg. Infect. Dis., 15:859 (2009).
Belser et al., J. Virol., 91:e00672 (2017).
Belser et al., Proc. Natl. Acad. Sci. USA, 105:7558 (2008).
Berkow et al., eds., The Merck Manual, 16th edition, Merck & Co., Rahway, N.J. (1992).
CDC, MMWR. Morb. Mortal Wkly. Rep., 53:284 (2004).
Centers for Disease Control and Prevention, H5N1 genetic changes inventory:a tool for influenza surveillance and preparedness. 2012 [cited 2017 Jul. 27]. https://www.cdc.gov/flu/pdf/avianflu/h5n1-inventory.pdf
Connor et al., Virology, 205:17 (1994).
Crawford et al., Science, 310:482 (2005).
Driskell et al., Vet. Pathol., 50:39 (2013).
Enami et al., Proc. Natl. Acad. Sci. U.S.A., 87:3802 (1990).
Fan et al., (2009) Virology, 384:28 (2009).
Felsenstein et al., Evolution, 39:783 (1985).
Fiorentini et al., Zoonoses Public Health, 58:573 (2011).
Fouchier et al., Proc. Natl. Acad. Sci, USA, 101:1356 (2004).
Gambaryan et al., Virology. 232:345 (1997).
Grand and Skehel, Nature, New Biology, 238:145 (1972).
Harder et al., Vet. Immunol. Immunopathol., 134:54 (2010).
Imai et al., Nature, 486:420 (2012).
Kilbourne, Bull. M2 World Health Org., 41: 653 (1969).
Koopmans et at, Lancet, 363:587 (2004).
Kuiken et al., Science, 306:241 (2004).
Kumar et al., Mol. Biol. Evol., 33:1870 (2016).
Laver & Webster, Virology, 69:511 (1976).
Lee et al., 2016. Emerg. Infect. Dis., 23:365 (3016)-367.
Lee et al., Vaccine, 29:4003 (2011).
Lee et al., Vaccine, 31:3268 (2013).
Lee et al., Vet. Rec., 171:477 (2012).
Li et al., J. Virol., 79:12058 (2005).
Matrosovich et al., J. Virol., 73:1146 (1999).
Matrosovich et al., Virology. 233:224 (1997),
Murphy, Infect. Dis. Clin. Pract., 2: 174 (1993).
Muster et al., Proc. Natl. Acad. Sci. USA, 88: 5177 (1991).
Myers, Overview of avian influenza surveillance in the United States. US Department of Agriculture, Animal and Plant Health inspection Service. 2015 [cited 2017 Feb. 12]. https://www.aphis. usda.gov/animal_health/animal_dis_spec/poultry/downloads/mtg/presentations/we 3_myers_ai_surveillance_overview.pdf
Osol (ed.), Remington's Pharmaceutical Sciences, Mack Publishing Co., Easton, Pa. 1324-1341 (1980).
Ostrowsky et al., Emerg. Infect. 18:1128 (2012).
Pappas et al., Clin. Vaccine Immunol., 14:1425 (2007).
Park et al., Proc. R. Soc. London B., 271:1547 (2004).
Robertson et al., Biologicals, 20:213 (1992).
Robertson et al., Giornale di Igiene e Medicina Preventiva, 29:4 (1988).
Said et al., J. Vet. Med. Sci., 73:541 (2011).
Saitou et al., Mol. Biol. Evol., 4:406 (1987).
Schmolke et al., PLoS Pathog., 7:e1002186 (2011).
Serene et al., In: Schrijver R S and Koch G, editors. Proceedings of the Frontis Workshop on Avian Influenza: Prevention and Control. Wageningen (the Netherlands); Springer; p. 19 (2003(a)).
Senne et al., In: Schrijver R S and Koch G, editors. Proceedings of the Frontis Workshop on Avian influenza: Prevention and Control. Wageningen (the Netherlands): Springer; p. 41 (2003(b)).
Song et al., Emerg. Infect. Dis., 14:741 (2008).
Spackman et al., J. Virol., 77:13399 (2003).
Subbarao et al., J. Virol., 67:7223 (1993).
Tamura, Mol. Biol. Evol., 9:678 (1992).
Thongratsakul et al., Asian Pac. J. Allergy Immunol., 28:294 (2010).
van Riel et al., Am. J. Pathol., 177:2185 (2010).
Wang et al., Acta Vet. Hung., 61:537 (2013).
Watanabe et al., Nature, 501:551 (2013).
Weinstein et al., J. Biol. Chem., 257:13845 (1982).
World Health Organization, Influenza at the human-animal interface. 2016 [cited 2017 Feb. 12]. http://www.who.int/inflenza/human_animal_interface/Influenza_Summary_IRA_HA_interface_12_19_2016.pdf?ua=1
Wright et al., editors. Fields Virology. Sixth ed. Philadelphia, Pa., USA: Lippincott Williams & Wilkins, p. 1186-243 (2013).
Xie et al., Acta Vet. Hung., 64:125 (2016).
Yang et al., J. Virol., 87:12433 (2013).
Yang et al., PLoS Pathog., 6:e1001081 (2010).
Yang et al., Science, 317:825 (2007).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A vaccine comprising an effective amount of an isolated influenza virus comprising a viral HA segment with sequences for a HA-1 having greater than 92% amino acid sequence identity to HA-1 encoded by a nucleotide sequence having one of SEQ ID Nos. 45-52, 54-64, 85, 93, 101, 109, or 117, or over 99% amino acid sequence identity to HA-1 sequences in one of SEQ ID Nos. 125, 137, 149, 161 or 173.

2. The vaccine of claim 1 wherein the influenza virus has a residue in the HA-1 at position 84 is not T (threonine), at position 104 is not G (glycine) or R (arginine), at position 109 is not G, D (aspartic acid) or S (serine), at position 125 is not A (alanine) or T, at position 180 is not S or T, at position 183 is not T, at position 188 is not S, at position 203 is not S, at position 292 is not T, or any combination thereof.

3. The vaccine of claim 1 wherein the influenza virus has a residue in HA-1 at position 84 that is N (asparagine) or Q (glutamine), at position 104 that is K (lysine), R or H (histidine), at position 109 that is N or E (glutamic acid), at position 125 that is S, at position 180 that is N or Q, at position 183 is I (isoleucine), L (leucine) or G, at position 188 is N or Q, at position 203 that is P (proline), at position 292 that is I, L or G, or any combination thereof.

4. The vaccine of claim 1 wherein the influenza virus has a residue at position 127 that is not N, or a residue at position 156 that is not T, or both.

5. The vaccine of claim 1 wherein the influenza virus has a residue at position 4, 36, 86, 93, 138, 151, 158, 177, 258, 269, 292, or any combination that is serine, alanine, valine, isoleucine, glycine or threonine, and/or a residue at position 290 that is proline, serine, alanine, valine, isoleucine, glycine or threonine.

6. The vaccine of claim 1 further comprising a different isolated influenza virus or antigen of a non-influenza microbial pathogen.

7. The vaccine of claim 1 wherein the isolated influenza virus is an attenuated virus.

8. The vaccine of claim 1 wherein the isolated influenza virus is a reassortant virus.

9. The vaccine of claim 1 which is modified by chemical, physical or molecular means.

10. The vaccine of claim 1 further comprising an adjuvant.

11. The vaccine of claim 1 further comprising a pharmaceutically acceptable carrier.

12. The vaccine of claim 11 wherein the carrier is suitable for intranasal or intramuscular administration.

13. The vaccine of claim 1 which is in freeze-dried form.

14. A method to prepare influenza virus, comprising: contacting an avian or mammalian cell with an isolated influenza virus comprising a viral HA segment with sequences for a HA-1 having greater than 92% amino acid sequence identity to a polypeptide encoded by a nucleotide sequence having one of SEQ ID Nos. 45-52 or 54-64 or over 99% amino acid sequence identity to HA-1 sequences in one of SEQ ID Nos. 125, 137, 149, 161 or 173.

15. The method of claim 14 further comprising isolating the virus.

16. The method of claim 14 wherein the cell is in an embryonated egg, or is a feline cell or a MDCK cell.

17. A method to immunize a mammal against influenza, comprising administering to the mammal a composition comprising an effective amount of isolated influenza virus comprising a viral HA segment with sequences for a HA-1 having greater than 92% amino acid sequence identity to HA-1 encoded by a nucleotide sequence having one of SEQ ID Nos. 45-52, 54-64, 85, 93, 101, 109, or 117, or over 99% amino acid sequence identity to HA-1 sequences in one of SEQ ID Nos. 125, 137, 149, 161 or 173.

18. The method of claim 17 wherein the mammal is a human or a feline.

19. The method of claim 17 wherein the administration is intranasal, intramuscular, subcutaneous, ocular or oral.

20. The method of claim 17 wherein the viral segment has sequences for a HA-1 having over 99% amino acid sequence identity to HA-1 sequences in one of SEQ ID Nos. 125, 137, 149, 161 or 173.


SEQUENCE LISTING (184 sequences)

SEQ ID NO: 1 - DNA, 252 bp, Inflluenza Virus
  misc_feature (1)...(252): n = A,T,C or G
acagaggtgcatgcatggtcagctcnagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgannnnnnnnnnnnnnnnnnnnnnnnnnnnnnnnnnnnnnnnnnnnnnnnnnnnnnnnnnnnnnnnnnnnnnnnn

SEQ ID NO: 2 - DNA, 230 bp, Inflluenza Virus
  misc_feature (1)...(230): n = A,T,C or G
gacaaggtcaatggattggacannnctcagttgccatgatgggaaagcatggttacacgtttgtgtcactggggaagatagaaatgcgactgctagtttcatttacaatggaatgcttgttgacagcattggttcatggtctcaaaacatcctcagaacccaggagtcagaatgcgtttgagnnnnnnnnnnnnnnnnnnnnnnnnnnnnnnnnnnnnnnnnnnnnnnnn

SEQ ID NO: 3 - DNA, 204 bp, Inflluenza Virus
  misc_feature (1)...(204): n = A,T,C or G
cccgaaaagggcagcgatagatcannctcannnttgccatgatgnnaaancatgnttacacnntttgtgtcactggggaagatagaaatgcgactgctagtttcatttacaatggaatgcttgttgacagcattggttcatggtctcaaaacatcctcagaacccaggagtcagaatgcgtttgagnnnnnnnnnnnnnnnnnn

SEQ ID NO: 4 - DNA, 203 bp, Inflluenza Virus
  misc_feature (1)...(203): n = A,T,C or G
tgaagagggcatgcgttgatcanctcannttnncctgatgnnaaancatgnttacacntttgtgtcactgnnnaaaatanaaatgcnactgctantttcatttacaatggaatgcttgttgacagtattggttcatggtctcaaaacatcctcagaacccaggagtcagaatgcgtttgagnnnnnnnnnnnnnnnnnnnnnn

SEQ ID NO: 5 - DNA, 202 bp, Inflluenza Virus
  misc_feature (1)...(202): n = A,T,C or G
gccaaagggcaagcgattgatcannctcannttgcccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgagnnnnnnnnnnnnnnnnnn

SEQ ID NO: 6 - DNA, 245 bp, Inflluenza Virus
  misc_feature (1)...(245): n = A,T,C or G
gcgccaaggtcaagccattgatcagctcagttgccatgatgggaaagcatggttacacgtttgtgtcactggggaagatagaaatgcgactgctagtttcatttacaatggaatgcttgttgacagcattggttcatggtctcaaaacatcctcagaacccaggagtcagaatgcgtttgagnnnnnnnnnnnnnnnnnnnnnnnnnnnnnnnnnnnnnnnnnnnnnnnnnnnnnnnnnnnnnnn

SEQ ID NO: 7 - DNA, 149 bp, Inflluenza Virus
  misc_feature (1)...(149): n = A,T,C or G
tnccntgatggnnnnnnntggttacacgtttgtgtcactggggaagatanaaatgcgactgctagtttcatttacaatggaatgcttgttgacagtattggttcatggtctcaaaacatcctcagaacccaggagtcagaatgcgtttg

SEQ ID NO: 8 - DNA, 186 bp, Inflluenza Virus
  misc_feature (1)...(186): n = A,T,C or G
gaaaagtgacatccattggatcagctcagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgannnnnn

SEQ ID NO: 9 - DNA, 242 bp, Inflluenza Virus
  misc_feature (1)...(242): n = A,T,C or G
ccttccaagggcagcgtttggacnnctcacnttnccatnatcncaaancatgcttacacctttgtctcactgnaaaatatataaatgctactgctaatttcatttacaatgaaatgcttgttgacagcattggttcatggtctcaaaacatcctcagaacccaggagtcagaatgcgtttgagnntnnntttngnnnnnnnnnnnnnnnnnnntnnnnnnnntnnnnnnnnnnnntngnnnn

SEQ ID NO: 10 - DNA, 202 bp, Inflluenza Virus
  misc_feature (1)...(202): n = A,T,C or G
gaaagctaggttttgagacatgaccaatacntgtcacaagcattccattgtaaatgaaactagcagtcgcatttctatcttccccagtgacacaaacgtgtaaccatgctttcccatcatggcaacttgagctggaccatgctatgcaaacttgtttggttcccaagtgaaacggaaaannnnnnnnnnnnnnnnnnnnnnn

SEQ ID NO: 11 - DNA, 202 bp, Inflluenza Virus
  misc_feature (1)...(202): n = A,T,C or G
actaacgtatgntaagagacatgaccaatgctgtcaacagcattccattgtaaatgaaactagcagtcgcatttctatcttccccagtgacacaaacgtgtaaccatgctttcccatcatggcaacttgagctggaccatgctatgcaaacttgtttggttcccaaatgaaacggaaaannnnnnnnnnnnnnnnnnnnnnn

SEQ ID NO: 12 - DNA, 202 bp, Inflluenza Virus
  misc_feature (1)...(202): n = A,T,C or G
gaagacggtacggtaagagacatgnaccaatgctgntcacaagcattccattgtaaatgaaactagcagtcgcatttctatcttccccagtgacacaaacgtgtaaccatgctttcccatcatggcaacttgagctggaccatgctatgcaaacttgtttggttcccaaatgaaacggaaaannnnnnnnnnnnnnnnnnnn

SEQ ID NO: 13 - DNA, 202 bp, Inflluenza Virus
  misc_feature (1)...(202): n = A,T,C or G
ctctaaaacgtacggtaaggacatgnaccaatacntgtcacaagcattccattgtaaatgaaactagcagtcgcatttctatcttccccagtgacacaaacgtgtaaccatgctttcccatcatggcaacttgagctggaccatgctatgcaaacttgtttggttcccaaatgaaacggaaaannnnnnnnnnnnnnnnnnn

SEQ ID NO: 14 - DNA, 202 bp, Inflluenza Virus
  misc_feature (1)...(202): n = A,T,C or G
gcagacgtacggttaggaacatgnnaccatagctgtcacaagcattccattgtaaatgaaactagcagtcgcatttctatcttccccagtgacacaaacgtgtaaccatgctttcccatcatggcaacttgagctggaccatgctatgcaaacttgtttggttcccaaatgaaacggaaaannnnnnnnnnnnnnnnnnnnn

SEQ ID NO: 15 - DNA, 202 bp, Inflluenza Virus
  misc_feature (1)...(202): n = A,T,C or G
gcggaggtatgttaagagacatgnaccaatgcntgtcacaagcattccattgtaaatgaaactagcagtcgcatttctatcttccccagtgacacaaacgtgtaaccatgctttcccatcatggcaacttgagctggaccatgctatgcaaacttgtttggttcccaaatgaaacggaaaannnnnnnnnnnnnnnnnnnnn

SEQ ID NO: 16 - skipped

SEQ ID NO: 17 - DNA, 205 bp, Inflluenza Virus
  misc_feature (1)...(205): n = A,T,C or G
gcaaagttacggtaagagacatgnaccaatacntgtcacaagcattccattgtaaatgaaactagcagtcgcatttctatcttccccagtgacacaaacgtgtaaccatgctttcccatcatggcaacttgagctggaccatgctatgcaaacttgtttggttcccaaatgaaacggaaaannnnnnnnnnnnnnnnnnnnnnnn

SEQ ID NO: 18 - DNA, 204 bp, Inflluenza Virus
  misc_feature (1)...(204): n = A,T,C or G
cccttaacgtaatggaaggacatgnnaccatgctgtcacaagcattccattgtaaatgaaactagcagtcgcatttctatcttccccagtgacacaaacgtgtaaccatgctttcccatcatggcaacttgagctggaccatgctatgcaaacttgtttggttcccaaatgaaacggaaaannnnnnnnnnnnnnnnnnnnnnn

SEQ ID NO: 19 - DNA, 153 bp, Inflluenza Virus
  misc_feature (1)...(153): n = A,T,C or G
tcgcgcgtcntctcgttactgggcacggtgagcgtgaaacaaatcctaaaattcccttagtcagaggtgacaggattggtcttgtctttagccattccatgaannnnnnnnnnnnnnnnnnnnnnnnnnnnnnnnnnnnnnnnnnnnnnnnnn

SEQ ID NO: 20 - DNA, 78 bp, Inflluenza Virus
tcatggaatggctaaagacaagaccaatcctgtcacctctgactaagggaattttaggatttgtattcacgctcaccg

SEQ ID NO: 21 - DNA, 152 bp, Inflluenza Virus
  misc_feature (1)...(152): n = A,T,C or G
tttcgctgtcntctcgttactgggcacggtgagcgtgaatcaaatcctaaaattcccttagtcagaggtgacaggattggtcttgtctttagccactccatgannnnnnnnnnnnnnnnnnnnnnnnnnnnnnnnnnnnnnnnnnnnnnnnn

SEQ ID NO: 22 - DNA, 103 bp, Inflluenza Virus
  misc_feature (1)...(103): n = A,T,C or G
ctcgcagtcntctcgttactgggcacggtgagcgtgaataaaatcctaaaattcccttagtcagaggtgacaggattggtcttgtctttagccactccatgnn

SEQ ID NO: 23 - DNA, 107 bp, Inflluenza Virus
  misc_feature (1)...(107): n = A,T,C or G
tccgccgtcntctcgttactgggcacggtgagcgtgaatcaaatcctaaaattcccttagtcagaggtgacaggattggtcttgtctttagccactccatgannnnn

SEQ ID NO: 24 - DNA, 152 bp, Inflluenza Virus
  misc_feature (1)...(152): n = A,T,C or G
ctacccgtcantctcgttactgggcacggtgagcgtgaatcaaatcctaaaattcccttagtcagaggtgacaggattggtcttgtctttagccactccatgaannnnnnnnnnnnnnnnnnnnnnnnnnnnnnnnnnnnnnnnnnnnnnnn

SEQ ID NO: 25 - DNA, 113 bp, Inflluenza Virus
  misc_feature (1)...(113): n = A,T,C or G
tcgccgtcantctcgttcctgggcacggtgagcgtgaaacaaatcctaaaattcccttagtcagaggtgacaggattggtcttgtctttagccactccatgannnnnnnnnnn

SEQ ID NO: 26 - DNA, 37 bp, Inflluenza Virus
  misc_feature (1)...(37): n = A,T,C or G
catggantggctaaagacaagaccaatccngtcacct

SEQ ID NO: 27-39 - skipped (13 entries)

SEQ ID NO: 40 - DNA, 1385 bp, Inlfuenza virus
agtaaaaatgaacccaaatcagaagataataacaattggctccgtctctctaatcattgcaacagtatgcttcctcatgcagattgccattctagcaatgactgtgacactgcatttcaagcaacatgaatgcagcatttcctaacagtcaggtagtgccacatgaaccaactacagagaaagaggtctgttcgaaaggagtagactataggagttggtcaaagccgcagtgtcaaattacaggatttgctcctttttccaaggacaattcaattcgactttctgctggtggaaacatttggataacacgagagccttatgtgtcgtgtgataccagcaaatgttaccaatttgcattggacaggggaccacattggataacaaacattcaaacggaacaatacatgatagtcccccatcggacccttttgatgaatgaactgggtgttccatttcacttgggaaccaaacaagtttgcatagcatggtccagctcaagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgcatcaatgggtcttgtacagtagtaatgactgacggaagtgcctcagggaaggctgacactagaatattatttgtcaaagagggaaagattgttcacattagcccattatcaggaagtgctcagcatatagaagaatgttcctgttatccccgctacccaaacgtcagatgtgtctgcagggacaactggaagggctctaataggcctactatagacataaacatgacagattatagcattgactccagttatgtgtgctcaggactcgttggggatacaccaagaaatgatgatagttctagcagcagcaattgcagggatcctaatgaagagaggggaaaccaaggtgtgaaaggatgggcctttgacagtggagatgatgtttggatgggtagaacaatcagtagagattcgcggtcaggctatgagacatttagggtcattggtggttggaccactaccaattccaagtcacagacaaatagacaagtcatagttgataataacaattggtctggttattctggtattttctctgttgaacataaaagctgtatcaataggtgtttttatgtggagttaataagaggaaggccgaaagaaaccagagtatggtggacctcaaacagtattgttgtgttttgtggcacttcaggcacttatggaacaggctcatggcctgatggggcaaatatcaatttcatgcctatatgagctttcgcaattttagaaaaa

SEQ ID NO: 41 - DNA, 1414 bp, Inlfuenza virus
agcaaaagcaggagtgaaaatgaatccaaatcagaagataataacaattggctccgtctctctaatcattgcaacagtatgtttcctcatgcagattgccattctagcaatgactgtaacactgcatttcaagcaacatgaatgcagcatttcctaacagtcaggtagtgccatgtgaaccaactacagagaaagaggtctgttcgaaagtagtagacatagaagctggtcaaagccgcagtgtcaaattacaggatttgctcctttttccaaggacaactcaattcgactttctgctggtggagacatttggataacaagagagccttatgtgtcgtgtgacaccagcaaatgttaccaatttgcacttgggcaggggaccacactggataacaaacattcaaacggaacaatacatgatagtcccccatcggacccttttgatgaatgaactgggtgttccatttcacttgggaaccaaacaagtttgcatagcatggtccagctcaagttgccatgatgggaaagcatggttgcacgtttgtgtcactggggatgatagaaatgcaactgctagtttcatttacaatgggatgcttgttgacagtattggttcatggtctcaaaatatcctcaggacccaggagtcagaatgcgtttgcatcaatgggtcttgtacagtagtaatgactgatggaagtgcctcagggaaggccgacactagaatattattcgtcaaagagggaaagattgttcacattagcccattgtcaggaagtgctcagcatatagaggaatgttcctgttatccccgatacccaaacgtcagatgtgtctgcagggacaactggaagggctctaataggcctgttatagacataaacatgacagattatagcatcgactccagttatgtgtgctcaggactcgttggggacacaccaaggaatgaggatagttctagcagcagcaactgcagggatcctaatgaagagaggggaaacccaggagtgaaaggatgggcctttgacagtggagatgatgtttggatgggtagaacaatcagtagggattcgcggtcaggctatgagacatttagggtcattggtggttggaccactgccaattccaagtcacagaccagcagacaagtcatagttgataataacaattggtctggttattctggtattttctctgttgaacacaaaagctgtatcaataggtgtttttatgtggagttaataagaggaaggccgaaagaaactagagtatggtggacctcaaacagtattgtcgtgttttgtggcacttcaggcacttatggaacaggctcatggcctgatggggcgaacatcaatttcatgcctatataagctttcgcaattttagaaaaaaactccttgtttctact

SEQ ID NO: 42 - DNA, 1003 bp, Inlfuenza virus
aggtagatgtttaaagatgagccttctaaccgaggtcgaaacgtacgttctctctatcatcccatcaggccccctcaaagccgagatcgcgcagagacttgaagatgtctttgcagggaagaacacagatcttgaggcactcatggaatggctaaagacaagaccaatcctgtcacctctgactaagggaattttaggatttgtattcacgctcaccgtgcccagtgaacgaggactgcagcgtagacgctttgtccagaatgctcttaatgggaatggagatccaaataacatggacagagcaatcaaactgtacaggaaattaaaaagggaaattacattccatggggccaaagaggtagcactcagttattccactggtgcacttgcaagttgcatgggccttatatacaacagaatgggaactgtgaccactgaaggggcatttggcctggtgtgcgccacgtgtgagcagattgctgactcccagcatcggtctcacagacagatggtgacaacaaccaacccactgatcagacatgagaacaggatggtactggctagtactacagctaaagccatggaacaggtggcaggatcaagcgagcaagcagcagaggctatggaggttgccagtcaggctaagcagatggtgcaagcgatgagaaccattgggactcatcctagctccagtaccggtctaagagatgatcttctgaaaaattgcaggcctatcaaaaaaggatgggagtgcaactgcatcgattcaagtgatcctctcgtcattgccgcaagtatccttggaatcttgcacttgatattgtggattcttgatcgcctttttttcaaatgcgtttatcgtcgccttaaatacggttgaaaagagggccttctacggaaggagtgcctgagtctatgagggaggaatatcggcaggaacagcagagtgctgtagatgttgacgatggtcattttgtcaacatagagctggagtaaaaaacta

SEQ ID NO: 43 - DNA, 993 bp, Inlfuenza virus
tagatgtttaaagatgagtcttctaaccgaggtcgaaacgtacgttctctctatatcccatcaggccccctcaaagccgagatcgcgcagagattgaagatgtctttgcagggaagaacacagatcttgaggcactcatggaatggctaaagacaagaccaatcctgtcacctctgactaaggggattttaggatttgtgttcacgctcaccgtgcccagtgagcggggactgcagcgtagcgctttgtccaaaatgctcttaatgggaatggggatccaaacaacatggacagggcagtcaaactgtacaggaaactaaaaagggaaattacattccatggggccaaagaggtagcactcagttattccactggtgcacttgccagttgcatgggccttatatacaacagaatgggaactgtgaccactgaaggggcatttggcctggtgtgcgccacgtgtgagcagattgctgactcccagcatcggtctcacagacagatggtgacgacaaccaacccactgatcagacatgagaacaggatggtactggctagtactactgctaaagccatggaacaggtagcagggtcaagcgaacaagcagcagaggctatggaggttgccagtcaggctaggcagatggtgcaggcaatgaggaccatcgggactcatcctagctccagtgctggtctaaaagatgatcttcttgaaaatttgcaggcctatcagaaacggatgggagtgcaaatgcagcgattcaagtgatcctctcgtcattgccgcaaatatcattgggatcttgcacttgatattgtggattcttgatcgcctttttttcaaatgcgtttatcgtcgccttaaatacgggttgaaaagagggccttctacggaaggggtgcctgagtctatgagggaggaatatcggcaggaacagcagagtgctgtggatgttgacgatggtcattttgtcaacatagagctggagtaaa

SEQ ID NO: 44 - DNA, 1654 bp, Inlfuenza virus
aaaatgaacattcaaatgctggtattcattgcttgtgtgctgactggagctagaggagacaaaatatgtcttgggcaccatgctgtggcaaatggaacaaaagtgaacacattaacagagaggggttgaagtagtgaatgccacagagacggtggaaactgtgaatatcaaaaaaatgtactcaagggaaaaggccaacagatctaggacaatgtggacttctaggaaccctaaggacctccccaatgtgatcaattcctggagttttcttctgatttgataattgaacgaagaaggaaacgatatatgctatcccggtaaattcataaatgaagaatcactgaggcagacttcgaaaatcaggaggaattaataaggagtcaatgggcttcacctatagtggaataaaccaatggatcaacaagtgcctgcacaagatcaggttcttctttctatacagagatgatggttgctgtcgaattcagacaatgcattattcccacaaatgacaaagtcgtatagaaatcccagaaacaaaccagctctgataatttggggagttcatcattctggatcagttaatgcagatcaaactctatggaaatggaaacaagttgataacagtaagaagctcaaaatacccaaccattcaccccaagtccgggagcacggagaatcgattttcactggctactccttgatcccaatgacacagtgaccttcactttcaatggagcattcatagcccctgacagggcaagttctttagaggagaatcactaggaatccagagtgatgctcctttggattccagttgtggggggattgctttcacagtgggggtacgatagtcagttccctgccattccaaaacatcaaccctagaattgtggggaaatgccctcggtatgtcaaacaaaaaagcctccttctagccacggaatgagaaatgttccagagaaaccaaagcccagaggcctttttggagcaattgctggattcatagagaatggatgggagggtctcatcaatggatggtatggtttcagacatcaaaatgcacaaggagaaggaactgcagccgactacaaaagcacccagtctgcaatagatcagatcacaggcaaattgaatcgtctaattggcaaaacaaatcagcaatttgagctgatagacaagagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaacac

SEQ ID NO: 45 - DNA, 523 bp, Inlfuenza virus
  misc_feature (1)...(523): n = A,T,C or G
ctaattggcaaancaaatcagcaatttgagctgatnaacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaacacactaa

SEQ ID NO: 46 - DNA, 501 bp, Inlfuenza virus
  misc_feature (1)...(501): n = A,T,C or G
caataagagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgnaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaaaacacccttgtttctactaata

SEQ ID NO: 47 - DNA, 424 bp, Inlfuenza virus
cgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaaaacacccttgtttctactaataca

SEQ ID NO: 48 - DNA, 509 bp, Inlfuenza virus
ctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaaaacacccttgtttctactaataca

SEQ ID NO: 49 - DNA, 483 bp, Inlfuenza virus
  misc_feature (1)...(483): n = A,T,C or G
gtggtatggtttcagacatcaaaatgtggtatggtttcagacatcaaaatgtggtatggtttcagacatcaaaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacatnatcttatggtttagcttcggggcatcatgttttcttcttctagccattgcaatggga

SEQ ID NO: 50 - DNA, 575 bp, Influenza Virus
  misc_feature (1)...(575): n = A,T,C or G
tggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgnaattgagtagtggatacaaagacataatcttatggtttagcttcggggcatcatgttttcttcttctagccattgcaatgggattggttttcatttgcataaagaa

SEQ ID NO: 51 - DNA, 578 bp, Influenza Virus
gggtggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaa

SEQ ID NO: 52 - DNA, 572 bp, Influenza Virus
tatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaa

SEQ ID NO: 53 - DNA, 1676 bp, Influenza Virus
aaaatgaacattcaaatgctggtattcattgcttgtgtgctgactggagctagaggagacaaaatatgtcttgggcaccatgctgtggcaaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctatcccggtaaattcataaatgaagaatcactgaggcagatccttcgaaaatcaggaggaattaataaggagtcaatgggcttcacctatagtggaataagaaccaatggatcaacaagtgcctgcacaagatcaggttcttctttctatacagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagaaggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaacac

SEQ ID NO: 54 - DNA, 864 bp, Influenza Virus
  misc_feature (1)...(864): n = A,T,C or G
cattaacagagagaggggttgaagtantgaatgccacaganacggtggaaactgtgaatatcaaaaaaatatgtactcaagggaaaaggccaacagatctaggacaatgtggacttctaggaaccctaanaggacctccccaatgtgatcaattcctggagttttcttctgatttgataattgaacgaagagaaggaaacgatatatgctatcccggtaaattcataaatgaagaatcactgaggcagatccttcgaaaatcaggaggaattaataaggagtcaatgggcttcacctatagtggaataagaaccaatggatcaacaagtgcctgcacaagatcaggttcttctttctatacagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatg

SEQ ID NO: 55 - DNA, 878 bp, Influenza Virus
tgctgtggcaaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatccttcgaaaatcaggaggaattagtaaggagtcaatgggcttcacctatagtggaataagaaccaatggatcaacaagtgcctgcacaagatcaggttcttctttctatgcagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagcca

SEQ ID NO: 56 - DNA, 877 bp, Influenza Virus
tgctgtggcaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatcttcgaaaatcaggaggaattagtaaggagtcaatgggcttcacctatagtggaataagaaccaatggatcaacaagtgcctgcacaagatcaggttcttctttctatgcagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccac

SEQ ID NO: 57 - DNA, 898 bp, Influenza Virus
  misc_feature (1)...(898): n = A,T,C or G
tagaggagacaaaatatgtcttgggcaccatgctgtggcaaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatccttcgaaaatcaggaggaattagtaaggagtcaatgggcttcacctatagtggaataagaaccaatggatcaacaagtgcctgcacaagatcaggttcttctttctatgcagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattcnaaaacatcaaccctagaattgtggggaaatgccctcggtatgtcaaacaaaaaagcctc

SEQ ID NO: 58 - DNA, 902 bp, Influenza Virus
gagctagaggagacaaaatatgtcttgggcaccatgctgtggcaaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatccttcgaaaatcaggaggaattagtaaggagtcaatgggcttcacctatagtggaataagaaccaatggatcaacaagtgcctgcacaagatcaggttcttctttctatgcagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctc

SEQ ID NO: 59 - DNA, 846 bp, Influenza Virus
  misc_feature (1)...(846): n = A,T,C or G
agctagaggagacaaaatatgtcttgggcaccatgctgtggcaaatgnaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatccttcgaaaatcaggaggaattagtaaggagtcaatgggcttcacctatagtggaataagaaccaatggatcaacaagtgcctgcacaagatcaggttcttctttctatgcagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcncantgggggtacgatagtcagttccctgccattccaaaa

SEQ ID NO: 60 - DNA, 804 bp, Influenza Virus
  misc_feature (1)...(804): n = A,T,C or G
gaccttcactttcaatggancattcatagcccctgacagggcaagtttctttagaggagaatcactaggaatccagagtgatgctcctttggattccagttgtggaggggattgctttcacagtgggggtacgatagtcagttccctgccnttccaaaacatcaaccctagaattgtggggaaatgccctcggtatgtcaaacaaaaaagcctccttctagccacaggaatgagaaatgttccagagaaaccaaagcccanaggcctttttggagcaattgctggattcatagagaatggatgggagggtctcatcaatggatggtatggtttcagacatcaaaatgcacaaggagaaggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcancatacaatanatcttacggactcagaaatgancaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtgg

SEQ ID NO: 61 - DNA, 852 bp, Influenza Virus
  misc_feature (1)...(852): n = A,T,C or G
ctggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgnaattgagtagtggatacaaagacataatctt

SEQ ID NO: 62 - DNA, 787 bp, Influenza Virus
cagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatctta

SEQ ID NO: 63 - DNA, 850 bp, Influenza Virus
  misc_feature (1)...(850): n = A,T,C or G
ctggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgnaattgagtagtggatacaaagacataatc

SEQ ID NO: 64 - DNA, 850 bp, Influenza Virus
tcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggataga

SEQ ID NO: 65 - DNA, 2337 bp, Influenza Virus
aaagcaggtcaagtatattcaatatggagagaataaaagaattaagagatctaatgtcacagtctcgcactcgcgagatactaactaaaaccactgtggaccatatggccataattaaaaaatacacgtcaggaagacaagagaagaaccctgcactcagaatgaagtggatgatggcaatgaaatatccaattacggcagataaaagaataatggagatggtcccagagaggaatgagcaaggacaaaccctctggagcaaaacaaatgatgccggttcagacagagtgatggtatcacctctggctgtgacatggtggaatagaaatggaccaacaacaagtacaattcactacccaaaggtctacaaaacctactttgaaaaagttgaaagattgaaacatgggacatttggcccagtccacttcaggaaccaagtcaaaataagacgaagagttgacataaaccctggccatgcagacctcagtgccaaagaggcacaaaatgtaatcatggaagttgttttcccaaatgaggtgggagcaagaatactaacatcagaatcacagctgacaataacaaaagaaaagaaggaggagctccaagattgtaaaattgcccccttgatggtagcatacatgttagaaagagagttggttcgcaaaacgagattcctcccagtggctggtggaacaagcagtgtatatattgaagtcttgcacttgactcaggggacatgctgggagcagatgtacaccccaggagggggggtgagaaatgatgatgttgatcaaagtttggttattgctgccagaaatatagtcagaagagcaacagtatcagcagacccactagcatctttattggagatgtgccacagtacacagattggtggaataaaaatggtagacattcttaggcagaatccaacagaggaacaagctgtggatatatgcagggctgcaatgggactgagaattagctcttctttcagctttggtggattcaccttcaaaagaacgagtggatcatcagtaaaaagagaagaagaagtgcttacgggcaaccttcaaacacttaaaataagggtgcatgaggggtatgaagaattcacaatggttggaagaagagcaacagctattctcagaaaagcaaccaggagattaattcaattaatagtaagtgggagagatgagcagtcgatagctgaagcaataatagtagccatggtattttcacaagaggactgcatgataaaggcagttcgaggtgatctgaattttgtcaatagggcaaatcagcgactgaaccccatgcatcaactcttgagacatttccaaaaggatgcaaaagtgctcttccagagctgggggattgaacaaatcgacagtgtaatgggaatgattggaatactgccagatatgaccccaaacactgaggtgtcactacgaggaataagagtcagcaaaatgggagtagatgaatactccagcacagaaagagtggtggtaagcattgaccgttttttaagggttagggacccacgggggaacatactattgtcacccgaggaagttagcgaaacacaagggacagaaaagttgacaataacttactcatcatcaatgatgtgggagatcaatggtcctgagtcagtgttggtcaacacctatcagtggatcatcagaaactgggagactgtgaaaatccaatggtcacaggaccccacaatgttgtacaataagatggaatttgagccatttcagtctctagttcctaaggctgctagaggccaatacagtggattcgtgagaacactgtttcaacaaatgcgagatgtacttggaacttttgacacatttcagataataaaacttctcccctttgctgctgccccaccggagcaaagtaggatgcagttctcctccctaactgtaaatgtaagaggatcaggaatgaggatactggtgagaggtaattctccagtattcaattacaataaggcaaccaagaggcttacaattctcgggaaagatgcaggtgcattaactaacgacccagatgaaggctcagctggagttgagtctgctgttctgagaggatttctcattttaggcaaagaagacaggagatatggcccagcattgagcatcaatgaactaagcaatcttgcaaaaggagagaaggctaacgtgttaattgggcaaggagacgtggtgttggtaatgaaacggaaacgggactctagcatacttactgacagtcagacagcgaccaaaaggattcggatggccatcaattagtgtcgaactgtttaaaaacgaccttgtttctact

SEQ ID NO: 66 - DNA, 2335 bp, Influenza Virus
agcaggcaaaccatttgaatggatgtcaatccgactttacttttcttgaaagttccagcgcaaaatgccataagcaccacattcccatatactggagatcctccatacagccatggaacggggacaggatacacaatggacacagtcagtagaacccatcaatactcagaaaaggggaagtggacaacaaacacagaaactggagcaccccaacttaacccaattgatggaccactgcccgaggataatgagccaagtggatatgcacaaacagactgtgtccttgaagcaatggctttccttgaagagtcccaccctggaatctttgaaaactcatgtcttgagacaatggaaattgtacaacaaacaagagtggacaaactgacccaaggtcgtcagacctatgattggacattaaacaggaatcagccggctgcaactgcgttagctaatactatagaagtcttcagatcgaacggcttaacaattaatgaatcaggaaggctaatagatttcctcaaggatgtgatggaatcaatggacaaagaggaactgagaataacaacgcatttccaacgaaaaagaagggtgagggacaacataaccaaaaagatgatcacacaaaggacaataggaaagaagaaacagaggctaaacaagaagagctacctaataagagcattgacactgaacacaatgacgaaagacgctgaaagaggcaaattaaagaggagagcaattgcaacacccgggatgcaaatcaggggatttgtgtattttgttgaaacattagcgagaagcatttgtgagaagcttgaacaatctggacttcctgttggaggcaatgaaaaaaaggctaaattagcaaatgttgtgagaaagatgatgactaattcacaagactcagaactctctttcacaatcactggagacaacactaaatggaatgaaaatcagaaccctaggatgtttctggcaatgataacatacataacaagaaaccaatctgaatggtttagaaatgtcttgagcattgcacctataatgttttcaaataaaatggcaaggttagggaaagggtacatgttcgagagtaaaagcatgaagcttcggacacaaataccagcagaaatgctagcaagcattgacctaaaatatttcaatgagtcaacaaagaagaaaatagagaagataaggcctcttctaatagatggcacagcctcattgagccctggaatgatgatgggaatgttcaacatgttaagtacagttttgggagtatcgatcctaaatctaggacagaagagatacaccaaaacaacatattggtgggacgggctccaatcatccgatgattttgctctcatagtgaatgctccggatcatgagggaatacaagcaggagtagatagattctataggacttgcaagctagtcgggatcaacatgagcaaaaagaagtcatacataaacaggacaggaacatttgagttcacaagctttttctatcgctatggatttgtagccaacttcagcatggagctgcccagctttggagtatctgggattaatgaatcggccgacatgagcattggagtaacagtgataaagaacaacatgataaacaatgaccttgggccagcaacagctcaaatggctcttcagctgttcatcaaggactatcgatacacgtaccgatgtcacagaggggacacacaaattcagacaaggaggtcattcgaactgaagaagttgtgggaacaaacccgctcaaaggcaggattgctagtttcagatgggggaccaaacttgtacaatattcggaatctccacattccagaagtttgcatgaaatgggagctaatggacgaagattatcagggaagactctgtaatcccctgaacccctttgttagccataaagagatagagtctgtaaacaatgcggtggtgatgccatctcatggcccagccaaaaacatggaatatgatgctgttgctactacgcattcctggattcctaaaaggaaccgctccatcctcaacacgagccaaagaggaatccttgaagatgaacagatgtaccaaaagtgctgcaatctattcgagaaattctttcctagcagttcatacaggagaccggttgggatctctagtatggtggaggctatggtttctagggcccgaattgatgctcgaattgacttcgaatctggacggattaagaaagaggagtttgctgagatcatgaagatctgttccaccattgaagaactcagacggcaaagatagtgaatttagcttgtccttcatgaaaaaatgccttgtttctact

SEQ ID NO: 67 - DNA, 2228 bp, Influenza Virus
agcaaaagcaggtactgattcaaaatggaagactttgtgcgacagtgcttcaatccaatgattgtcgagcttgcggaaaaggcaatgaaagaatatggggagaatccgaaaatcgaaactaacaaatttgctgcaatatgcactcacctggaagtatgcttcatgtattcggatttccatttcattaacgaacggggcgaatcaataatcgtagagtctggtgatccaaatgcattactgaagcatcgatttgagataattgaagggagagaccgaacaatagcctggacagtagtgaatagtatctgcaacaccacgggggtagagaagcctaagttccttccagatctgtatgattacaaggaaaatcgattcattgaaattggagtgacacgaagggaggttcacatatactatctagagaaagccaacaagataaaatctgagatgacacacattcacattttctcattcactggagaggaaatggccaccaaagcggactacacactagacgaagagagtagggcaagaataaaaaccagactgttcactataagacaagaaatggccagcaggggcttatgggattcctttcgtcagtccgaaagaggcgaagatacaattgaagaaaaatttgaaatcacaggaaccatgcgcaggcttgccgaccaaagtcttccaccgaacttctccagccttgaaaattttagagcctatgtggatggatttaaaccgaacggctgcattgagggaaagctttctcaaatgtcaaaagaagtgaacgcaagaattgaaccatttttgaagacaacaccacgccctctcaaattaccagatggccctccttgctctcaacgatcgaaattcttgctgatggatgccttaaaattaagcattgaagatccgagtcatgagggagaaggaatacctctgtatgatgcaatcaaatgcttgaaaacattttttggctggaaagagcctaacatagtcaagccacatgagaaaggcataaatcccaattacctcctggcttggaaacaggtgctagcagaacttcaagacattgaaaatgatgaaaagatcccaaagacaaagaacatgagaaaaacgagccaattgaagtgggcacttggtgaaaacatggcaccagaaaaggtagacttcgaggactgcaaggatgttagcgatctaaaacagtataatagtgatgagccaaagtctagatctctagcaagttggatccagagtgaattcaataaggcatgtgaattgaccgattcaagttggatagagcttgatgaaataggggaggatattgccccaattgagtacattgcaagtatgaggaggaactattttacagcagaagtgtcccattgtagggctactgaatacataatgaaaggagtgtatataaacacagctttgctcaatgcatcttgtgctgccatggatgattttcagctgatcccaatgataagcaaatgcaggataaaagaagggaggcggaaaacaaacctgtatgggttcattataaaaggaaggtctcatttgagaaatgacactgacgtggtaaactttgtgagcatggaattttctctcactgacccgagactggaaccacacaaatgggagagatactgtgttcttgaaataggagatatgctcctaaggactgcaataggccaggtatcaaggcccatgttcttgtatgttagaaccaatggaacttccaaaattaagatgaagtggggtatggagatgaggcgctgccttcttcaatctcttcagcagattgagagcatgattgaggcagagtcgtccgtcaaagaaaaagacatgacaaaagaattctttgaaaataaatctgagacatggccaattggagagtcacccaagggggtggaagagggctccatcggaaaagtgtgcaggaccttgctggcaaaatctgtattcaacagtctgtatgcatctccacaactcgaggggttctcagctgaatcaaggaaactgcttctcattgttcaggcacttagggacaacctggaacctggaaccttcgatcttggagggctatatgaagcaattgaggagtgcctgattaatgatccctgggttttgcttaatgcatcttggtttaactccttcctcacacatgcactgagatagttgtggcaatgctactgtttgctatccatactgtccaaaaaagtaccttgttt

SEQ ID NO: 68 - DNA, 1676 bp, Influenza Virus; identical to SEQ ID NO: 53

SEQ ID NO: 69 - DNA, 1561 bp, Influenza Virus
aaagcagggtagataatcactcaccgagtgacatccacatcatggcgtctcaaggcaccaaacgatcttatgagcagatggaaactggaggggaacgccagaatgccactgagatcagaacatctgttgggaaaatggttggtggaatcgggagattctacatacagatgtgtactgaactcaaactcagtgactatgaaggaagactgatccaaaacagcataacaatagagagaatggttctctcggcatttgatgagagaagaaatagatatctggaagagcatcccagtgctggaaaagaccctaagaaaactggaggtccaatctacaggaggagagatgggaagtgggtgagagaattgatcctgtatgacaaagaggaaatcaggagaatttggcgtcaagcgaataatggagaagatgcgactgctggtctcacccatttgatgatatggcattccaatcttaatgatgccacatatcagaggacaagggcacttgtgcgcagtgggatggaccccagaatgtgctctctgatgcaaggctcgactcttccgaggagatctggagcggccggagcagcagtaaaaggagttggaacaatggtgatggaattgatccggatgatcaaacgaggaatcaatgatcggaatttctggagaggcgaaaatggacggagaacaagaattgcttatgaaagaatgtgcaacattctcaaggggaaattccaaacagcagcacaacgagcaatgatggaccaggtgagagaaagccggaatcctgggaatgctgaaattgaggatctcatattcctggcacgatctgccctcattctgagaggatcagtggctcacaaatcctgtctgcctgcttgtgtatatggacttgctgtggccagtggatacgattttgaaagagagggatactccctagtcggaattgatcctttccgcctgcttcaaaacagtcaagtcttcagtcttatcaggcctaacgaaaatccagcacataaaagtcaactggtatggatggcatgtcactctgcagcatttgaagatctgagagtgttaaacttcatcagaggaacaagagtggttccaagaggacaactgtccaccagaggaatccaagtcgcttcaaatgagaacatggaaacgatggattccagcactctcgaattgagaagtagatactgggctataagaaccagaagtggaggaaacacaaatcagcagagagcatccgcagggcaaatcagcgtacatccaacattctctgttcagagaaacctcccatttgagagagcaaccattatggcggcatttacaggaaacactgaaggcagaacttcagacatgagaactgagatcataaggatgatggaaaatgccaaacctgaagatgtgtctttccaagggcggggagtattcgagctctcggacgaaaaggcaacgagcccgatcgtgccttcctttgacatgagtaacgaaggatcttatttcttcggagacaatgcagaggagtatgacaattgaagaaaaatacccttgtttctact

SEQ ID NO: 70 - DNA, 1404 bp, Influenza Virus
aaaaaaatgaacccaaatcagaagataataacaattggctccgtctctctaatcattgcaacagtatgcttcctcatgcagattgccattctagcaatgactgtgacactgcatttcaagcaacatgaatgcagcatttccaataacagtcaggtagtgccacatgaaccaactacagagaaagaggtctgttcgaaaggagtagactataggagttggtcaaagccgcagtgtcaaattacaggatttgctcctttttccaaggacaattcaattcgactttctgctggtggaaacatttggataacacgagagccttatgtgtcgtgtgataccagcaaatgttaccaatttgcacttggacaggggaccacattggataacaaacattcaaacggaacaatacatgatagagtcccccatcggacccttttgatgaatgaactgggtgttccatttcacttgggaaccaaacaagtttgcatagcatggtccagctcaagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgcatcaatgggtcttgtacagtagtaatgactgacggaagtgcctcagggaaggctgacactagaatattatttgtcaaagagggaaagattgttcacattagcccattatcaggaagtgctcagcatatagaagaatgttcctgttatccccgctacccaaacgtcagatgtgtctgcagggacaactggaagggctctaataggcctactatagacataaacatgacagattatagcattgactccagttatgtgtgctcaggactcgttggggatacaccaagaaatgatgatagttctagcagcagcaattgcagggatcctaatgaagagaggggaaaccaaggtgtgaaaggatgggcctttgacagtggagatgatgtttggatgggtagaacaatcagtagagattcgcggtcaggctatgagacatttagggtcattggtggttggaccactaccaattccaagtcacagacaaatagacaagtcatagttgataataacaattggtctggttattctggtattttctctgttgaacataaaagctgtatcaataggtgtttttatgtggagttaataagaggaaggccgaaagaaaccagagtatggtggacctcaaacagtattgttgtgttttgtggcacttcaggcacttatggaacaggctcatggcctgatggggcaaatatcaatttcatgcctatatgagctttcgcaattttagaaaaactccttgtttctact

SEQ ID NO: 71 - DNA, 1023 bp, Influenza Virus
aaagcaggtagatgtttaaagatgagccttctaaccgaggtcgaaacgtacgttctctctatcatcccatcaggccccctcaaagccgagatcgcgcagagacttgaagatgtctttgcagggaagaacacagatcttgaggcactcatggaatggctaaagacaagaccaatcctgtcacctctgactaagggaattttaggatttgtattcacgctcaccgtgcccagtgaacgaggactgcagcgtagacgctttgtccagaatgctcttaatgggaatggagatccaaataacatggacagagcaatcaaactgtacaggaaattaaaaagggaaattacattccatggggccaaagaggtagcactcagttattccactggtgcacttgcaagttgcatgggccttatatacaacagaatgggaactgtgaccactgaaggggcatttggcctggtgtgcgccacgtgtgagcagattgctgactcccagcatcggtctcacagacagatggtgacaacaaccaacccactgatcagacatgagaacaggatggtactggctagtactacagctaaagccatggaacaggtggcaggatcaagcgagcaagcagcagaggctatggaggttgccagtcaggctaagcagatggtgcaagcgatgagaaccattgggactcatcctagctccagtaccggtctaagagatgatcttcttgaaaaattgcaggcctatcaaaaaaggatgggagtgcaactgcatcgattcaagtgatcctctcgtcattgccgcaagtatccttggaatcttgcacttgatattgtggattcttgatcgcctttttttcaaatgcgtttatcgtcgccttaaatacgggttgaaaagagggccttctacggaaggagtgcctgagtctatgagggaggaatatcggcaggaacagcagagtgctgtagatgttgacgatggtcattttgtcaacatagagctggagtaaaaaactaccttgtttctact

SEQ ID NO: 72 - DNA, 849 bp, Influenza Virus
ataatggattccaacacgataacctcgtttcaggtagactgttatctatggcacataagaaagctactcagcatgagagacatgtgtgatgctccctttgatgatagaatcagaagagatcaaaaagcattaaagggaagaagcagcacacttgggctcgacctgcgagtggccacaatggaaggcaaaaagattgttgaagacatcataaagagtgaaactgatgaaaagctaagaattgcaattgcatccagccctgcacctcggtacattactgatatgagcatagaggaaataagtagggaatggtacatgctaatgccaaggcagaagatgacagggggtctgatggtgaaaatggatcaggccattatgaacaagaggataatgcttaaagcaaacttctctgtcctatttgatcaactggaaacactagtctcactgagggctttcacagacgatggagccattgtagctgaaatatgtcccattccttctatgccaggaagttctgcagaggatgtcaaagatgcaattggaatcctcatcggtggacttgaatggaatgataactcagttcgaacatctgaaaatatacagagattcgcttggggagtccgtgatgagaataggggacctccactctcttcaaaacagaaacgctacatggagagaagagttgagtcagaaatttgaagagatcagatggctgattgcagaatgcagaaacatattaaccaaaactgaaaatagcttcgagcaaataacgttcttgcaagcattgcaactcttacttgaagttgagagtgagataagaacattttcttttcagcttatttagtactaaaa

SEQ ID NO: 73 - protein, 759 aa, Influenza Virus
MERIKELRDLMSQSRTREILTKTTVDHMAIIKKYTSGRQEKNPALRMKWMMAMKYPITADKRIMEMVPERNEQGQTLWSKTNDAGSDRVMVSPLAVTWWNRNGPTTSTIHYPKVYKTYFEKVERLKHGTFGPVHFRNQVKIRRRVDINPGHADLSAKEAQNVIMEVVFPNEVGARILTSESQLTITKEKKEELQDCKIAPLMVAYMLERELVRKTRFLPVAGGTSSVYIEVLHLTQGTCWEQMYTPGGGVRNDDVDQSLVIAARNIVRRATVSADPLASLLEMCHSTQIGGIKMVDILRQNPTEEQAVDICRAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQSWGIEQIDSVMGMIGILPDMTPNTEVSLRGIRVSKMGVDEYSSTERVVVSIDRFLRVRDPRGNILLSPEEVSETQGTEKLTITYSSSMMWEINGPESVLVNTYQWIIRNWETVKIQWSQDPTMLYNKMEFEPFQSLVPKAARGQYSGFVRTLFQQMRDVLGTFDTFQIIKLLPFAAAPPEQSRMQFSSLTVNVRGSGMRILVRGNSPVFNYNKATKRLTILGKDAGALTNDPDEGSAGVESAVLRGFLILGKEDRRYGPALSINELSNLAKGEKANVLIGQGDVVLVMKRKRDSSILTDSQTATKRIRMAIN

SEQ ID NO: 74 - protein, 757 aa, Influenza Virus
MDVNPTLLFLKVPAQNAISTTFPYTGDPPYSHGTGTGYTMDTVSRTHQYSEKGKWTTNTETGAPQLNPIDGPLPEDNEPSGYAQTDCVLEAMAFLEESHPGIFENSCLETMEIVQQTRVDKLTQGRQTYDWTLNRNQPAATALANTIEVFRSNGLTINESGRLIDFLKDVMESMDKEELRITTHFQRKRRVRDNITKKMITQRTIGKKKQRLNKKSYLIRALTLNTMTKDAERGKLKRRAIATPGMQIRGFVYFVETLARSICEKLEQSGLPVGGNEKKAKLANVVRKMMTNSQDSELSFTITGDNTKWNENQNPRMFLAMITYITRNQSEWFRNVLSIAPIMFSNKMARLGKGYMFESKSMKLRTQIPAEMLASIDLKYFNESTKKKIEKIRPLLIDGTASLSPGMMMGMFNMLSTVLGVSILNLGQKRYTKTTYWWDGLQSSDDFALIVNAPDHEGIQAGVDRFYRTCKLVGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFELKKLWEQTRSKAGLLVSDGGPNLYNIRNLHIPEVCMKWELMDEDYQGRLCNPLNPFVSHKEIESVNNAVVMPSHGPAKNMEYDAVATTHSWIPKRNRSILNTSQRGILEDEQMYQKCCNLFEKFFPSSSYRRPVGISSMVEAMVSRARIDARIDFESGRIKKEEFAEIMKICSTIEELRRQR

SEQ ID NO: 75 - protein, 90 aa, Influenza Virus
MERGQDTQWTQSVEPINTQKRGSGQQTQKLEHPNLTQLMDHCPRIMSQVDMHKQTVSLKQWLSLKSPTLESLKTHVLRQWKLYNKQEWTN

SEQ ID NO: 76 - protein, 716 aa, Influenza Virus
MEDFVRQCFNPMIVELAEKAMKEYGENPKIETNKFAAICTHLEVCFMYSDFHFINERGESIIVESGDPNALLKHRFEIIEGRDRTIAWTVVNSICNTTGVEKPKFLPDLYDYKENRFIEIGVTRREVHIYYLEKANKIKSEMTHIHIFSFTGEEMATKADYTLDEESRARIKTRLFTIRQEMASRGLWDSFRQSERGEDTIEEKFEITGTMRRLADQSLPPNFSSLENFRAYVDGFKPNGCIEGKLSQMSKEVNARIEPFLKTTPRPLKLPDGPPCSQRSKFLLMDALKLSIEDPSHEGEGIPLYDAIKCLKTFFGWKEPNIVKPHEKGINPNYLLAWKQVLAELQDIENDEKIPKTKNMRKTSQLKWALGENMAPEKVDFEDCKDVSDLKQYNSDEPKSRSLASWIQSEFNKACELTDSSWIELDEIGEDIAPIEYIASMRRNYFTAEVSHCRATEYIMKGVYINTALLNASCAAMDDFQLIPMISKCRIKEGRRKTNLYGFIIKGRSHLRNDTDVVNFVSMEFSLTDPRLEPHKWERYCVLEIGDMLLRTAIGQVSRPMFLYVRTNGTSKIKMKWGMEMRRCLLQSLQQIESMIEAESSVKEKDMTKEFFENKSETWPIGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 77 - protein, 252 aa, Influenza Virus
MEDFVRQCFNPMIVELAEKAMKEYGENPKIETNKFAAICTHLEVCFMYSDFHFINERGESIIVESGDPNALLKHRFEIIEGRDRTIAWTVVNSICNTTGVEKPKFLPDLYDYKENRFIEIGVTRREVHIYYLEKANKIKSEMTHIHIFSFTGEEMATKADYTLDEESRARIKTRLFTIRQEMASRGLWDSFVSPKEAKIQLKKNLKSQEPCAGLPTKVFHRTSPALKILEPMWMDLNRTAALRESFLKCQKK

SEQ ID NO: 78 - protein, 552 aa, Influenza Virus
MNIQMLVFIACVLTGARGDKICLGHHAVANGTKVNTLTERGVEVVNATETVETVNIKKICTQGKRPTDLGQCGLLGTLIGPPQCDQFLEFSSDLIIERREGNDICYPGKFINEESLRQILRKSGGINKESMGFTYSGIRTNGSTSACTRSGSSFYTEMKWLLSNSDNALFPQMTKSYRNPRNKPALIIWGVHHSGSVNEQIKLYGNGNKLITVRSSKYQQPFTPSPGARRIDFHWLLLDPNDTVTFTFNGAFIAPDRASFFRGESLGIQSDAPLDSSCGGDCFHSGGTIVSSLPFQNINPRIVGKCPRYVKQKSLLLATGMRNVPEKPKPRGLFGAIAGFIENGWEGLINGWYGFRHQNAQGEGTAADYKSTQSAIDQITGKLNRLIGKTNQQFELIDNEFSEIEQQIGNVINWTRDAMTEIWSYNAEMLVAMENQHTIDLTDSEMSKLYERVKKQLRENAEEDGTGCFEIFHKCDNHCMESIRNNTYDHTQYRTESLQNRIRIDPVKLSSGYKDIILWFSFGASCFLLLAIAMGLVFICIKNGNMQCTICI

SEQ ID NO: 79 - protein, 498 aa, Influenza Virus
MASQGTKRSYEQMETGGERQNATEIRTSVGKMVGGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRSGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVLNFIRGTRVVPRGQLSTRGIQVASNENMETMDSSTLELRSRYWAIRTRSGGNTNQQRASAGQISVHPTFSVQRNLPFERATIMAAFTGNTEGRTSDMRTEIIRMMENAKPEDVSFQGRGVFELSDEKATSPIVPSFDMSNEGSYFFGDNAEEYDN

SEQ ID NO: 80 - protein, 453 aa, Influenza Virus
MNPNQKIITIGSVSLIIATVCFLMQIAILAMTVTLHFKQHECSISNNSQVVPHEPTTEKEVCSKGVDYRSWSKPQCQITGFAPFSKDNSIRLSAGGNIWITREPYVSCDTSKCYQFALGQGTTLDNKHSNGTIHDRVPHRTLLMNELGVPFHLGTKQVCIAWSSSSCHDGKAWLHVCVTGEDRNATASFIYNGMLVDSIGSWSQNILRTQESECVCINGSCTVVMTDGSASGKADTRILFVKEGKIVHISPLSGSAQHIEECSCYPRYPNVRCVCRDNWKGSNRPTIDINMTDYSIDSSYVCSGLVGDTPRNDDSSSSSNCRDPNEERGNQGVKGWAFDSGDDVWMGRTISRDSRSGYETFRVIGGWTTTNSKSQTNRQVIVDNNNWSGYSGIFSVEHKSCINRCFYVELIRGRPKETRVWWTSNSIVVFCGTSGTYGTGSWPDGANINFMPI

SEQ ID NO: 81 - protein, 97 aa, Influenza Virus
MSLLTEVETPIKKGWECNCIDSSDPLVIAASILGILHLILWILDRLFFKCVYRRLKYGLKRGPSTEGVPESMREEYRQEQQSAVDVDDGHFVNIELE

SEQ ID NO: 82 - protein, 251 aa, Influenza Virus
MSLLTEVETYVLSIIPSGPLKAEIAQRLEDVFAGKNTDLEALMEWLKTRPILSPLTKGILGFVFTLTVPSERGLQRRRFVQNALNGNGDPNNMDRAIKLYRKLKREITFHGAKEVALSYSTGALASCMGLIYNRMGTVTTEGAFGLVCATCEQIADSQHRSHRQMVTTTNPLIRHENRMVLASTTAKAMEQVAGSSEQAAEAMEVASQAKQMVQAMRTIGTHPSSSTGLRDDLLEKLQAYQKRMGVQLHRF

SEQ ID NO: 83 - protein, 121 aa, Influenza Virus
MDSNTITSFQEVLQRMSKMQLESSSVDLNGMITQFEHLKIYRDSLGESVMRIGDLHSLQNRNATWREELSQKFEEIRWLIAECRNILTKTENSFEQITFLQALQLLLEVESEIRTFSFQLI

SEQ ID NO: 84 - protein, 230 aa, Influenza Virus
MDSNTITSFQVDCYLWHIRKLLSMRDMCDAPFDDRIRRDQKALKGRSSTLGLDLRVATMEGKKIVEDIIKSETDEKLRIAIASSPAPRYITDMSIEEISREWYMLMPRQKMTGGLMVKMDQAIMNKRIMLKANFSVLFDQLETLVSLRAFTDDGAIVAEICPIPSMPGSSAEDVKDAIGILIGGLEWNDNSVRTSENIQRFAWGVRDENRGPPLSSKQKRYMERRVESEI

SEQ ID NO: 85 - DNA, 1709 bp, Influenza virus
agcaaaagcaggggatacaaaatgaacattcaaatgctggtattcactgcttgtgtgctgactggagctagaggagacaaaatatgtcttgggcaccatgctgtggcaaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatccttcgaaaatcaggaggaattagtaaggagtcaatgggcttcacctatagtggaataagaaccaatggatcaacaagtgcctgcacaagatcaggttcttctttctatgcagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaaaacacccttgtttctact

SEQ ID NO: 86 - DNA, 1027 bp, Influenza virus
agcgaaagcaggtagatgtttaaagatgagccttctaaccgaggtcgaaacgtacgttctctctatcatcccatcaggccccctcaaagccgagatcgcgcagagacttgaagatgtctttgcagggaagaacacagatcttgaggcactcatggaatggctaaagacaagaccaatcctgtcacctctgactaagggaattttaggatttgtattcacgctcaccgtgcccagtgaacgaggactgcagcgtagacgctttgtccagaatgctcttaatgggaatggagatccaaataacatggacagagcaatcaaactgtacaggaaattaaaaagggaaattacattccatggggccaaagaggtagcactcagttattccactggtgcacttgcaagttgcatgggccttatatacaacagaatgggaactgtgaccactgaaggggcatttggcctggtgtgcgccacgtgtgagcagattgctgactcccagcatcggtctcacagacagatggtgacaacaaccaacccactgatcagacatgagaacaggatggtactggctagtactacagctaaagccatggaacaggtggcaggatcaagcgagcaagcagcagaggctatggaggttgccagtcaggctaagcagatggtgcaagcgatgagaaccattgggactcatcctagctccagtaccggtctaagagatgatcttcttgaaaaattgcaggcctatcaaaaaaggatgggagtgcaactgcatcgattcaagtgatcctctcgtcattgccgcaagtatccttggaatcttgcacttgatattgtggattcttgatcgcctttttttcaaatgcgtttatcgtcgccttaaatacgggttgaaaagagggccttctacggaaggagtgcctgagtctatgagggaggaatatcggcaggaacagcagagtgctgtagatgttgacgatggtcattttgtcaacatagagctggagtaaaaaactaccttgtttctact

SEQ ID NO: 87 - DNA, 1418 bp, Influenza virus
agcaaaagcaggagtaaaaaatgaacccaaatcagaagataataacaattggctccgtctctctaatcattgcaacagtatgctttctcatgcagattgccattctagcaatgactgtgacactgcatttcaagcaatatgaatgcagcatttccaataacagtcaggtagtgccacatgaaccaactacagagaaagaggtctgttcgaaaggagtagactataggagttggtcaaagccgcagtgtcaaattacaggatttgctcctttttccaaggacaattcaattcgactttctgctggtggaaacatttggataacaagagagccttatgtgtcgtgtgataccagcaaatgttaccaatttgcacttggacaggggaccacattggataacaaacattcaaacggaacaatacatgatagagtcccccatcggacccttttgatgaatgaactgggtgttccatttcacttgggaaccaaacaagtttgcatagcatggtccagctcaagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgcatcaatgggtcttgtacagtagtaatgactgacggaagtgcctcagggaaggctgacactagaatattatttgtcaaagagggaaagattgttcacattagcccattatcaggaagtgctcagcatatagaggaatgttcctgttatccccgctacccaaacgtcagatgtgtctgcagggacaactggaagggctctaataggcctactatagacataaacatgacagattatagcattgactccagttatgtgtgctcaggactcgttggggatacaccaagaaatgatgatagttctagcagcagcaattgcagggatcctaatgaagagaggggaaaccaaggtgtgaaaggatgggcctttgacagtggagatgatgtttggatgggtagaacaatcagtagagattcgcggtcaggctatgagacatttagggtcattggtggttggaccactaccaattccaagtcacagacaaatagacaagtcatagttgataataacaattggtctggttattctggtattttctctgttgaacataaaagctgtatcaataggtgtttttatgtggagttaataagaggaaggccgaaagaaaccagagtatggtggacctcaaacagtattgttgtgttttgtggcacttcaggcacttatggaacaggctcatggcctgatggggcaaatatcaatttcatgcctatatgagctttcgcaattttagaaaaactccttgtttctact

SEQ ID NO: 88 - DNA, 1565 bp, Influenza virus
agcaaaagcagggtagataatcactcaccgagtgacatccacatcatggcgtctcaaggcaccaaacgatcttatgagcagatggaaactggaggggaacgccagaatgccactgagatcagaacatctgttgggaaaatggttggtggaatcgggagattctacatacagatgtgtactgaactcaaactcagtgactatgaaggaagactgatccaaaacagcataacgatagagagaatggttctctcggcatttgatgagagaagaaatagatatctggaagagcatcccagtgctggaaaagaccctaagaaaactggaggtccaatctacaggaggagagatgggaagtgggtgagagaattgatcctgtatgacaaagaggaaatcaggagaatttggcgtcaagcgaataatggagaagatgcgactgctggtctcacccatttgatgatatggcattccaatcttaatgatgccacatatcagaggacaagggcacttgtgcgcagtgggatggaccccagaatgtgctctttgatgcaaggctcgactcttccgaggagatctggagcggccggagcagcagtaaaaggagttggaacaatggtgatggaattgatccggatgatcaaacgaggaatcaatgatcggaatttctggagaggcgaaaatggacggagaacaagaattgcttatgaaagaatgtgcaacattctcaaggggaaattccaaacagcagcacaacgagcaatgatggaccaggtgagagaaagccggaatcctgggaatgctgaaattgaggatctcatattcctggcacgatctgccctcattctgagaggatcagtggctcacaaatcctgtctgcctgcttgtgtatatggacttgctgtggccagtggatacgattttgaaagagagggatactccctagtcggaattgatcctttccgcctgcttcaaaacagtcaagtcttcagtcttatcaggcctaacgaaaatccagcacataaaagtcaactggtatggatggcatgtcactctgcagcatttgaagatctgagagtgttaaacttcatcagaggaacaagagtggttccaagaggacaactgtccaccagaggaatccaagtcgcttcaaatgagaacatggaaacgatggattccagcactcttgaattgagaagtagatactgggctataagaaccagaagtggaggaaacacaaatcagcagagagcatccgcagggcaaatcagcgtacatccaacattctctgttcagagaaacctcccatttgagagagcaaccattatggcggcatttacaggaaacactgaaggcagaacttcagacatgagaactgagatcataaggatgatggaaaatgccaaacctgaagatgtgtctttccaagggcggggagtattcgagctctcggacgaaaaggcaacgagcccgatcgtgccttcctttgacatgagtaacgaaggatcttatttcttcggagacaatgcagaggagtatgacaattgaagaaaaatacccttgtttctact

SEQ ID NO: 89 - DNA, 890 bp, Influenza virus
agcaaaagcagggtgacaaatacataatggattccaacacgataacctcgtttcaggtagactgttatctatggcacataagaaagctactcagcatgagagacatgtgtgatgctccctttgatgatagactcagaagagatcaaaaagcattaaagggaagaagcagcacacttgggctcgacctgcgagtggccacaatggaaggcaaaaagattgttgaagacatcataaagagtgaaactgatgaaaagctaagaattgcaattgcatccagccctgcacctcggtacattactgatatgagcatagaggaaataagtagggaatggtacatgctaatgccaaggcagaagatgacagggggtctgatggtgaaaatggatcaggccattatgaacaagaggataatgcttaaagcaaacttctctgtcctatttgatcaactggaaacactagtctcactgagggctttcacagacgatggagccattgtagctgaaatatgtcccattccttctatgccaggaagttctgcagaggatgtcaaagatgcaattggaatcctcatcggtggacttgaatggaatgataactcagttcgaacgtctgaaaatatacagagattcgcttggggagtccgtgatgagaataggggacctccactctcttcaaaacagaaacgctacatggagagaagagttgagtcagaaatttgatgagatcagatggctgattgcagaatgcagaaacatattaaccaaaactgaaaatagcttcgagcaaataacgttcttgcaagcattgcaactcttacttgaagttgagagtgagataagaacattttcttttcagcttatttagtactaaaaaacacccttgtttctact

SEQ ID NO: 90 - DNA, 2233 bp, Influenza virus
agcgaaagcaggtactgattcaaaatggaagactttgtgcgacagtgcttcaatccaatgattgtcgagcttgcggaaaaggcaatgaaagaatatggggagaatccgaaaatcgaaactaacaaatttgctgcaatatgcactcacctggaagtatgcttcatgtattcggatttccatttcattaacgaacagggcgaatcaataatcgtagagtctggtgatccaaatgcattactgaagcatcgatttgagataattgaagggagagaccgaacaatagcctggacagtagtgaatagtatctgcaacaccacgggggtagagaagcctaagttccttccagatctgtatgattacaaggaaaatcgattcattgaaattggagtgacacgaagggaggttcacatatactatctagagaaagccaacaagataaaatctgagatgacacacatccatattttctcattcactggagaggaaatggccaccaaagcggactatacactagacgaagagagtagggcaagaataaaaaccagactgttcactataagacaagaaatggccagcaggggcttatgggattcctttcgtcagtccgaaagaggcgaagatacaattgaagaaaaatttgaaatcacaggaaccatgcgcaggcttgccgaccaaagtcttccaccgaacttctccagccttgaaaattttagagcctatgtggatggatttaaaccgaacggctgcattgagggaaagctttctcaaatgtcaaaagaagtgaacgcaagaattgaaccatttttgaagacaacaccacgccctctcaaattaccagatggccctccttgctctcaacgatcgaaattcttgctgatggatgccttaaaattaagcattgaagatccgagtcatgagggagaaggaatacctctgtatgatgcaatcaaatgcttgaaaacattttttggctggaaagagcctaacatagtcaagccacatgagaaaggcataaatcccaattacctcctggcttggaaacaggtgctagcagaacttcaagacattgaaaatgatgaaaagatcccaaagacaaagaacatgagaaaaacgagccaattgaagtgggcacttggtgaaaacatggcaccagaaaaggtagacttcgaggactgcaaggatgttagcgatctaaaacagtataatagtgatgagccaaagtctagatctctagcaagttggatccagagtgaattcaataaggcatgtgaattgaccgattcaagttggatagagcttgatgaaataggggaggatattgccccaattgagtacattgcaagtatgaggaggaactattttacagcagaagtgtcccattgtagggctactgaatacataatgaaaggagtgtatataaacacagctttgctcaatgcatcttgtgctgccatggatgattttcagctgatcccaatgataagcaaatgcaggataaaagaagggaggcggaaaacaaacctgtatgggttcattataaaaggaaggtctcatttgagaaatgacactgacgtggtaaactttgtgagcatggaattttctctcactgacccgagactggaaccacacaaatgggagagatactgtgttcttgaaataggagatatgctcctaaggactgcaataggccaggtatcaaggcccatgttcttgtatgttagaaccaatggaacttccaaaattaagatgaagtggggtatggagatgaggcgctgccttcttcaatctcttcagcagattgagagcatgattgaggcagagtcgtccgtcaaagaaaaagacatgacaaaagaattctttgaaaataaatctgagacatggccaattggagagtcacccaagggggtggaagagggctccatcggaaaagtgtgcaggaccttgctggcaaaatctgtattcaacagtctgtatgcatctccacaactcgaggggttctcagctgaatcaaggaaactgcttctcattgttcaggcacttagggacaacctggaacctggaaccttcgatcttggagggctatatgaagcaattgaggagtgcctgattaatgatccctgggttttgcttaatgcatcttggtttaactccttcctcacacatgcactgagatagttgtggcaatgctactgtttgctatccatactgtccaaaaaagtaccttgtttctact

SEQ ID NO: 91 - DNA, 2341 bp, Influenza virus
agcgaaagcaggcaaaccatttgaatggatgtcaatccgactttacttttcttgaaagttccagcgcaaaatgccataagcaccacattcccatatactggagatcctccatacagccatggaacggggacaggatacacaatggacacagtcagtagaacccatcaatactcagaaaaggggaagtggacaacaaacacagaaactggagcaccccaacttaacccaattgatggaccactgcccgaggataatgagccaagtggatatgcacaaacagactgtgtccttgaagcaatggctttccttgaagagtcccaccctggaatctttgaaaactcatgtcttgagacaatggaaattgtacaacaaacaagagtggacaaactgacccaaggtcgtcagacctatgattggacattaaacaggaatcagccggctgcaactgcgttagctaatactatagaagtcttcagatcgaacggcctaacaattaatgaatcaggaaggctaatagatttcctcaaggatgtgatggaatcaatggacaaagaggaactgagaataacaacgcatttccaacgaaaaagaagggtgagggacaacataaccaaaaagatgatcacacaaaggacaataggaaagaagaaacagaggctaaacaagaagagctacctaataagagcattgacactgaacacaatgacgaaagacgctgaaagaggcaaattaaagaggagagcaattgcaacacccgggatgcaaatcaggggatttgtgtattttgttgaaacattagcgagaagcatttgtgagaagcttgaacaatctggacttcctgttggaggcaatgaaaaaaaggctaaattagcaaatgttgtgagaaagatgatgactaattcacaagactcagaactctctttcacaatcactggagacaacactaaatggaatgaaaatcagaaccctaggatgtttctggcaatgataacatacataacaagaaaccaatctgaatggtttagaaatgtcttgagcattgcacctataatgttttcaaataaaatggcaaggttagggaaagggtacatgttcgagagtaaaagcatgaagcttcggacacaaataccagcagaaatgctagcaagcattgacctaaaatatttcaatgagtcaacaaagaagaaaatagagaagataaggcctcttctaatagatggcacagcctcattgagccctggaatgatgatgggaatgttcaacatgttaagtacagttttgggagtatcgatcctaaatctaggacagaagagatacaccaaaacaacatactggtgggacgggctccaatcatccgatgattttgctctcatagtgaatgctccggatcatgagggaatacaagcaggagtagatagattctataggacttgcaagctagtcgggatcaacatgagcaaaaagaagtcatacataaacaggacaggaacatttgagttcacaagctttttctatcgctatggatttgtagccaacttcagcatggagctgcccagctttggagtatctgggattaatgaatcggccgacatgagcattggagtaacagtgataaagaacaacatgataaacaatgaccttgggccagcaacagctcaaatggctcttcagctgttcatcaaggactatcgatacacgtaccgatgtcacagaggggacacacaaattcagacaaggaggtcattcgaactgaagaagttgtgggaacaaacccgctcaaaggcaggattgctagtttcagatgggggaccaaacttgtacaatattcggaatctccacattccagaagtttgcatgaaatgggagctaatggacgaagattatcagggaagactctgtaatcccctgaacccctttgttagccataaagagatagagtctgtaaacaatgcggtggtgatgccatctcatggcccagccaaaaacatggaatatgatgctgttgctactacgcattcctggattcctaaaaggaaccgctccatcctcaacacgagccaaagaggaatccttgaagatgaacagatgtaccaaaagtgctgcaatctattcgagaaattctttcctagcagttcatacaggagaccggttgggatctctagtatggtggaggctatggtttctagggcccgaattgatgctcgaattgacttcgaatctggacggattaagaaagaggagtttgctgagatcatgaagatctgttccaccattgaagaactcagacggcaaagatagtgaatttagcttgtccttcatgaaaaaatgccttgtttctact

SEQ ID NO: 92 - DNA, 2341 bp, Influenza virus
agcgaaagcaggtcaagtatattcaatatggagagaataaaagaattaagagatctaatgtcacagtctcgcactcgcgagatactaactaaaaccactgtggaccatatggccataattaaaaaatacacgtcaggaagacaagagaagaaccctgcactcagaatgaagtggatgatggcaatgaaatatccaattacggcagataaaagaataatggagatggtcccagagaggaatgagcaaggacaaaccctctggagcaaaacaaatgatgccggttcagacagagtgatggtatcacctctggctgtgacatggtggaatagaaatggaccaacaacaagtacaattcactacccaaaggtctacaaaacctactttgaaaaagttgaaagattgaaacatgggacatttggcccagtccacttcaggaaccaagtcaaaataagacgaagagttgacataaaccctggccatgcagacctcagtgccaaagaggcacaaaatgtaatcatggaagttgttttcccaaatgaggtgggagcaagaatactaacatcagaatcacagctgacaataacaaaagaaaagaaggaggagctccaagattgtaaaattgcccccttgatggtagcatacatgttagaaagagagttggttcgcaaaacgagattcctcccagtggctggtggaacaagcagtgtatatattgaagtcttgcacttgactcaggggacatgctgggagcagatgtacaccccaggagggggggtgagaaatgatgatgttgatcaaagtttggttattgctgccagaaatatagtcagaagagcaacagtatcagcagacccactagcatctttattggagatgtgccacagtacacagattggtggaataaaaatggtagacattcttaggcagaatccaacagaggaacaagctgtggatatatgcagggctgcaatgggactgagaattagctcttctttcagctttggtggattcaccttcaaaagaacgagtggatcatcagtaaaaagagaagaagaagtgcttacgggcaaccttcaaacacttaaaataagggtgcatgaggggtatgaagaattcacaatggttggaagaagagcaacagctattctcagaaaagcaaccaggagattaattcaattaatagtaagtgggagagatgagcagtcgatagctgaagcaataatagtagccatggtattttcacaagaggactgcatgataaaggcagttcgaggtgatctgaattttgtcaatagggcaaatcagcgactgaaccccatgcatcaactcttgagacatttccaaaaggatgcaaaagtgctcttccagagctgggggattgaacaaatcgacagtgtaatgggaatgattggaatactgccagatatgaccccaaacactgaggtgtcactacgaggaataagagtcagcaaaatgggagtagatgaatactccagtacagaaagagtggtggtaagcattgaccgttttttaagggttagggacccacgggggaacatactattgtcacccgaggaagttagcgaaacacaagggacagaaaagttgacaataacttactcatcatcaatgatgtgggagatcaatggtcctgagtcagtgttggtcaacacctatcagtggatcatcagaaactgggagactgtgaaaatccaatggtcacaggaccccacaatgttgtacaataagatggaatttgagccatttcagtctctagttcctaaggctgctagaggccaatacagtggattcgtgagaacactgtttcaacaaatgcgagatgtacttggaacttttgacacatttcagataataaaacttctcccatttgctgctgccccaccggagcaaagtaggatgcagttctcctccctaactgtaaatgtaagaggatcaggaatgaggatactggtgagaggtaattctccagtattcaattacaataaggccaccaagaggcttacaattctcgggaaagatgcaggtgcattaactaacgacccagatgaaggctcagctggagttgagtctgctgttctgagaggatttctcattttaggcaaagaagacaggagatatggcccagcattgagcatcaatgaactaagcaatcttgcaaaaggagagaaggctaacgtgttaattgggcaaggagacgtggtgttggtaatgaaacggaaacgggactctagcatacttactgacagtcagacagcgaccaaaaggattcggatggccatcaattagtgtcgaactgtttaaaaacgaccttgtttctact

SEQ ID NO: 93 - DNA, 1709 bp, Influenza virus
agcaaaagcaggggatacaaaatgaacattcaaatgctggtattcactgcttgtgtgctgactggagctagaggagacaaaatatgtcttgggcaccatgctgtggcaaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatccttcgaaaatcaggaggaattagtaaggagtcaatgggcttcacctatagtggaataagaaccaatggatcaacaagtgcctgcacaagatcaggttcttctttctatgcagagatgaagtggttgctgtcgaactcagacaatgcattattcccacaaatgacaaaatcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctcggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaaaacacccttgtttctact

SEQ ID NO: 94 - DNA, 1027 bp, Influenza virus; identical to SEQ ID NO: 86

SEQ ID NO: 95 - DNA, 1419 bp, Influenza virus
agcaaaagcaggagtaaaaaaatgaacccaaatcagaagataataacaattggctccgtctctctaatcattgcaacagtatgcttcctcatgcagattgccattctagcaatgactgtgacactgcatttcaagcaacatgaatgcagcatttccaataacagtcaggtagtgccacatgaaccaactacagagaaagaagtctgttcgaaaggagtagactataggagttggtcaaagccgcagtgtcaaattacaggatttgctcctttttccaaggacaattcaattcgactttctgctggtggaaacatttggataacacgagagccttatgtgtcgtgtgataccagcaaatgttaccaatttgcacttggacaggggaccacattggataacaaacattcaaacggaacaatacatgatagagtcccccatcggacccttttgatgaatgaactgggtgttccatttcacttgggaaccaaacaagtttgcatagcatggtccagctcaagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgcatcaatgggtcttgtacagtagtaatgactgacggaagtgcctcagggaaggctgacactagaatattatttgtcaaagagggaaagattgttcacattagcccattatcaggaagtgctcagcatatagaagaatgttcctgttatccccgctacccaaacgtcagatgtgtctgcagggacaactggaagggctctaataggcctactatagacataaacatgacagattatagcattgactccagttatgtgtgctcaggactcgttggggatacaccaagaaatgatgatagttctagcagcagcaattgcagggatcctaatgaagagaggggaaaccaaggtgtgaaaggatgggcctttgacagtggagatgatgtttggatgggtagaacaatcagtagagattcgcggtcaggctatgagacatttagggtcattggtggttggaccactaccaattccaagtcacagacaaatagacaagtcatagttgataataacaattggtctggttattctggtattttctctgttgaacataaaagctgtatcaataggtgtttttatgtggagttaataagaggaaggccgaaagaaaccagagtatggtggacctcaaacagtattgttgtgttttgtggcacttcaggcacttatggaacaggctcatggcctgatggggcaaatatcaatttcatgcctatatgagctttcgcaattttagaaaaactccttgtttctact

SEQ ID NO: 96 - DNA, 1419 bp, Influenza virus; identical to SEQ ID NO: 95

SEQ ID NO: 97 - DNA, 890 bp, Influenza virus
agcaaaagcagggtgacaaatacataatggattccaacacgataacctcgtttcaggtagactgttatctatggcacataagaaagctactcagcatgagagacatgtgtgatgctccctttgatgatagactcagaagagatcaaaaagcattaaagggaagaagcagcacacttgggctcgacctgcgagtggccacaatggaaggcaaaaagattgttgaagacatcataaagagtgaaactgatgaaaagctaagaattgcaattgcatccagccctgcacctcggtacattactgatatgagcatagaggaaataagtagggaatggtacatgctaatgccaaggcagaagatgacagggggtctgatggtgaaaatggatcaggccattatgaacaagaggataatgcttaaagcaaacttctctgtcctatttgatcaactggaaacactagtctcactgagggctttcacagacgatggagccattgtagctgaaatatgtcccattccttctatgccaggaagttctgcagaggatgtcaaagatgcaattggaatcctcatcggtggacttgaatggaatgataactcagttcgaacgtctgaaaatatacagagattcgcttggggagtccgtgatgagaataggggacctccactctcttcaaaacagaaacgctacatggagagaagagttgagtcagaaatttgaagagatcagatggctgattgcagaatgcagaaacatattaaccaaaactgaaaatagcttcgagcaaataacgttcttgcaagcattgcaactcttacttgaagttgagagtgagataagaacattttcttttcagcttatttagtactaaaaaacacccttgtttctact

SEQ ID NO: 98 - DNA, 2233 bp, Influenza virus
agcgaaagcaggtactgattcaaaatggaagactttgtgcgacagtgcttcaatccaatgattgtcgagcttgcggaaaaggcaatgaaagaatatggggagaatccgaaaatcgaaactaacaaatttgctgcaatatgcactcacctggaagtatgcttcatgtattcggatttccatttcattaacgaacggggcgaatcaataatcgtagagtctggtgatccaaatgcattactgaagcatcgatttgagataattgaagggagagaccgaacaatagcctggacagtagtgaatagtatctgcaacaccacgggggtagagaagcctaagttccttccagatctgtatgattacaaggaaaatcgattcattgaaattggagtgacacgaagggaggttcacatatactatctagagaaagccaacaagataaaatctgagatgacacacattcacattttctcattcactggagaggaaatggccaccaaagcggactacacactagacgaagagagtagggcaagaataaaaaccagactgttcactataagacaagaaatggccagcaggggcttatgggattcctttcgtcagtccgaaagaggcgaagatacaattgaagaaaaatttgaaatcacaggaaccatgcgcaggcttgccgaccaaagtcttccaccgaacttctccagccttgaaaattttagagcctatgtggatggatttaaaccgaacggctgcattgagggaaagctttctcaaatgtcaaaagaagtgaacgcaagaattgaaccatttttgaagacaacaccacgccctctcaaattaccagatggccctccttgctctcaacgatcgaaattcttgctgatggatgccttaaaattaagcattgaagatccgagtcatgagggagaaggaatacctctgtatgatgcaatcaaatgcttgaaaacattttttggctggaaagagcctaacatagtcaagccacatgagaaaggcataaatcccaattacctcctggcttggaaacaggtgctagcagaacttcaagacattgaaaatgatgaaaagatcccaaagacaaagaacatgagaaaaacgagccaattgaagtgggcacttggtgaaaacatggcaccagaaaaggtagatttcgaggactgcaaggatgttagcgatctaaaacagtataatagtgatgagccaaagtctagatctctagcaagttggatccagagtgaattcaataaggcatgtgaattgaccgattcaagttggatagagcttgatgaaataggggaggatattgccccaattgagtacattgcaagtatgaggaggaactattttacagcagaagtgtcccattgtagggctactgaatacataatgaaaggagtgtatataaacacagctttgctcaatgcatcttgtgctgccatggatgattttcagctgatcccaatgataagcaaatgcaggataaaagaagggaggcggaaaacaaacctgtatgggttcattataaaaggaaggtctcatttgagaaatgacactgacgtggtaaactttgtgagcatggaattttctctcactgacccgagactggaaccacacaaatgggagagatactgtgttcttgaaataggagatatgctcctaaggactgcaataggccaggtatcaaggcccatgttcttgtatgttagaaccaatggaacttccaaaattaagatgaagtggggtatggagatgaggcgctgccttcttcaatctcttcagcagattgagagcatgattgaggcagagtcgtccgtcaaagaaaaagacatgacaaaagaattctttgaaaataaatctgagacatggccaattggagagtcacccaagggggtggaagagggctccatcggaaaagtgtgcaggaccttgctggcaaaatctgtattcaacagtctgtatgcatctccacaactcgaggggttctcagctgaatcaaggaaactgcttctcattgttcaggcacttagggacaacctggaacctggaaccttcgatcttggagggctatatgaagcaattgaggagtgcctgattaatgatccctgggttttgcttaatgcatcttggtttaactccttcctcacacatgcactgagatagttgtggcaatgctactgtttgctatccatactgtccaaaaaagtaccttgtttctact

SEQ ID NO: 99 - DNA, 2341 bp, Influenza virus
agcgaaagcaggcaaaccatttgaatggatgtcaatccgactttacttttcttgaaagttccagcgcaaaatgccataagcaccacattcccatatactggagatcctccatacagccatggaacggggacaggatacacaatggacacagtcagtagaacccatcaatactcagaaaaggggaagtggacaacaaacacagaaactggagcaccccaacttaacccaattgatggaccactacccgaggataatgagccaagtggatatgcacaaacagactgtgtccttgaagcaatggctttccttgaagagtcccaccctggaatctttgaaaactcatgtcttgagacaatggaaattgtacaacaaacaagagtggacaaactgacccaaggtcgtcagacctatgattggacattaaacaggaatcagccggctgcaactgcgttagctaatactatagaagtcttcagatcgaacggcttaacaattaatgaatcaggaaggctaatagatttcctcaaggatgtgatggaatcaatggacaaagaggaactgagaataacaacgcatttccaacgaaaaagaagggtgagggacaacataaccaaaaagatgatcacacaaaggacaataggaaagaagaaacagaggctaaacaagaagagctacctaataagagcattgacactgaacacaatgacgaaagacgctgaaagaggcaaattaaagaggagagcaattgcaacacccgggatgcaaatcaggggatttgtgtattttgttgaaacattagcgagaagcatttgtgagaagcttgaacaatctggacttcctgttggaggcaatgaaaaaaaggctaaattagcaaatgttgtgagaaagatgatgactaattcacaagactcagaactctctttcacaatcactggagacaacactaaatggaatgaaaatcagaaccctaggatgtttctggcaatgataacatacataacaagaaaccaatctgaatggtttagaaatgtcttgagcattgcacctataatgttttcaaataaaatggcaaggttagggaaagggtacatgttcgagagtaaaagcatgaagcttcggacacaaataccagcagaaatgctagcaagcattgacctaaaatatttcaatgagtcaacaaagaagaaaatagagaagataaggcctcttctaatagatggcacagcctcattgagccctggaatgatgatgggaatgttcaacatgttaagtacagttttgggagtatcgatcctaaatctaggacagaagagatacaccaaaacaacatactggtgggacgggctccaatcatccgatgattttgctctcatagtgaatgctccggatcatgagggaatacaagcaggagtagatagattctataggacttgcaagctagtcgggatcaacatgagcaaaaagaagtcatacataaacaggacaggaacatttgagttcacaagctttttctatcgctatggatttgtagccaacttcagcatggagctgcccagctttggagtatctgggattaatgaatcggccgacatgagcattggagtaacagtgataaagaacaacatgataaacaatgaccttgggccagcaacagctcaaatggctcttcagctgttcatcaaggactatcgatacacgtaccgatgtcacagaggggacacacaaattcagacaaggaggtcattcgaactgaagaagttgtgggaacaaacccgctcaaaggcaggattgctagtttcagatgggggaccaaacttgtacaatattcggaatctccacattccagaagtttgcatgaaatgggagctaatggacgaagattatcagggaagactctgtaatcccctgaacccctttgttagccataaagagatagagtctgtaaacaatgcggtggtgatgccatctcatggcccagccaaaaacatggaatatgatgctgttgctactacgcattcctggattcctaaaaggaaccgctccatcctcaacacgagccaaagaggaatccttgaagatgaacagatgtaccaaaagtgctgcaatctattcgagaaattctttcctagcagttcatacaggagaccggttgggatctctagtatggtggaggctatggtttctagggcccgaattgatgctcgaattgacttcgaatctggacggattaagaaagaggagtttgctgagatcatgaagatctgttccaccattgaagaactcagacggcaaagatagtgaatttagcttgtccttcatgaaaaaatgccttgtttctact

SEQ ID NO: 100 - DNA, 2341 bp, Influenza virus
agcgaaagcaggtcaagtatattcaatatggagagaataaaagaattaagagatctaatgtcacagtctcgcactcgcgagatactaactaaaaccactgtggaccatatggccataattaaaaaatacacgtcaggaagacaagagaagaaccctgcactcagaatgaagtggatgatggcaatgaaatatccaattacggcagataaaagaataatggagatggtcccagagaggaatgagcaaggacaaaccctctggagcaaaacaaatgatgccggttcagacagagtgatggtatcacctctggctgtgacatggtggaatagaaatggaccaacaacaagtacaattcactacccaaaggtctacaaaacctactttgaaaaagttgaaagattgaaacatgggacatttggcccagtccacttcaggaaccaagtcaaaataagacgaagagttgacataaaccctggccatgcagacctcagtgccaaagaggcacaaaatgtaatcatggaagttgttttcccaaatgaggtgggagcaagaatactaacatcagaatcacagctgacaataacaaaagaaaagaaggaggagctccaagattgtaaaattgcccccttgatggtagcatacatgttagaaagagagttggttcgcaaaacgagattcctcccagtggctggtggaacaagcagtgtatatattgaagtcttgcacttgactcaggggacatgctgggagcagatgtacaccccaggagggggggtgagaaatgatgatgttgatcaaagtttggttattgctgccagaaatatagtcagaagagcaacagtatcagcagacccactagcatctttattggagatgtgccacagtacacagattggtggaataaaaatggtagacattcttaggcagaatccaacagaggaacaagctgtggatatatgcagggctgcaatgggactgagaattagctcttctttcagctttggtggattcaccttcaaaagaacgagtggatcatcagtaaaaagagaagaagaagtgcttacgggcaaccttcaaacacttaaaataagggtgcatgaggggtatgaagaattcacaatggttggaagaagagcaacagctattctcagaaaagcaaccaggagattaattcaattaatagtaagtgggagagatgagcagtcgatagctgaagcaataatagtagccatggtattttcacaagaggactgcatgataaaggcagttcgaggtgatctgaattttgtcaatagggcaaatcagcgactgaaccccatgcatcaactcttgagacatttccaaaaggatgcaaaagtgctcttccagaactgggggattgaacaaatcgacagtgtaatgggaatgattggaatactgccagatatgaccccaaacactgaggtgtcactacgaggaataagagtcagcaaaatgggagtagatgaatactccagcacagaaagagtggtggtaagcattgaccgttttttaagggttagggacccacgggggaacatactattgtcacccgaggaagttagcgaaacacaagggacagaaaagttgacaataacttactcatcatcaatgatgtgggagatcaatggtcctgagtcagtgttggtcaacacctatcagtggatcatcagaaactgggagactgtgaaaatccaatggtcacaggaccccacaatgttgtacaataagatggaatttgagccatttcagtctctagttcctaaggctgctagaggccaatacagtggattcgtgagaacactgtttcaacaaatgcgagatgtacttggaacttttgacacatttcagataataaaacttctcccctttgctgctgccccaccggagcaaagtaggatgcagttctcctccctaactgtaaatgtaagaggatcaggaatgaggatactggtgagaggtaattctccagtattcaattacaataaggcaaccaagaggcttacaattctcgggaaagatgcaggtgcattaactaacgacccagatgaaggctcagctggagttgagtctgctgttctgagaggatttctcattttaggcaaagaagacaggagatatggcccagcattgagcatcaatgaactaagcaatcttgcaaaaggagagaaggctaacgtgttaattgggcaaggagacgtggtgttggtaatgaaacggaaacgggactctagcatacttactgacagtcagacagcgaccaaaaggattcggatggccatcaattagtgtcgaactgtttaaaaacgaccttgtttctact

SEQ ID NO: 101 - DNA, 1709 bp, Influenza virus
agcaaaagcaggggatacaaaatgaacattcaaatgctggtattcactgcttgtgtgctgactggagctagaggagacaaaatatgtcttgggcaccatgctgtggcaaatggaacaaaagtgaacacattaacagagagaggggttgaagtagtgaatgccacagagacggtggaaactgtgaatatcaaaaaaatatgtactcaagggaaaaggccaacagatctaggacaatgtggacttctaggaaccctaataggacctccccaatgtgatcaattcctggagttttcttctgatttgataattgaacgaagagaaggaaacgatatatgctaccccggtaaattcataaatgaagaatcactgaggcagatccttcgaaaatcaggaggaattagtaaggagtcaatgggcttcacctatagtggaataagaaccaatggatcaacaagtgcctgcacaagatcaggttcttctttctatgcagagatgaagtggttgctgtcgaattcagacaatgcattattcccacaaatgacaaagtcgtatagaaatcccagaaacaaaccagctctgataatttggggagttcatcattctggatcagttaatgagcagatcaaactctatggaaatggaaacaagttgataacagtaagaagctcaaaataccagcaaccattcaccccaagtccgggagcacggagaatcgattttcactggctactccttgatcccaatgacacagtgaccttcactttcaatggagcattcatagcccctgacagggcaagtttctttagaggagaatcactaggaatccagagtgatgctcctttggattccagttgtggaggggattgctttcacagtgggggtacgatagtcagttccctgccattccaaaacatcaaccctagaattgtggggaaatgccctaggtatgtcaaacaaaaaagcctccttctagccacaggaatgagaaatgttccagagaaaccaaagcccagaggcctttttggagcaattgctggattcatagagaatggatgggagggtctcatcaatggatggtatggtttcagacatcaaaatgcacaaggagagggaactgcagccgactacaaaagcacccagtctgcaatagatcagatcacaggcaaattgaatcgtctaattggcaaaacaaatcagcaatttgagctgatagacaatgagttcagtgaaatagaacaacaaataggaaatgtcattaattggacacgagacgcaatgacagagatatggtcgtataatgctgagatgttggtggcaatggaaaatcagcatacaatagatcttacggactcagaaatgagcaaactttatgagcgtgtcaaaaaacaactaagggagaatgctgaagaagatggaactggatgttttgagatattccataagtgtgataaccactgtatggagagcataaggaacaacacttatgaccatactcaatacagaacagagtcattacagaatagaatacggatagatccagtgaaattgagtagtggatacaaagacataatcttatggtttagcttcggggcatcatgttttcttcttctagccattgcaatgggattggttttcatttgcataaagaatggaaacatgcagtgcactatttgtatatagtttgagaaaaaaacacccttgtttctact

SEQ ID NO: 102 - DNA, 1027 bp, Influenza virus; identical to SEQ ID NO: 86

SEQ ID NO: 103 - DNA, 1418 bp, Influenza virus
agcaaaagcaggagtaaaaaatgaacccaaatcagaagataataacaattggctccgtctctctaatcattgcaacagtatgctttctcatgcagattgccattctagcaatgactgtgacactgcatttcaagcaatatgaatgcagcatttccaataacagtcaggtagtgccacatgaaccaactacagagaaagaggtctgttcgaaaggagtagactataggagttggtcaaagccgcagtgtcaaattacaggatttgctcctttttccaaggacaattcaattcgactttctgctggtggaaacatttggataacaagagagccttatgtgtcgtgtgataccagcaaatgttaccaatttgcacttggacaggggaccacattggataacaaacattcaaacggaacaatacatgatagagtcccccatcggacccttttgatgaatgaactgggtgttccatttcacttgggaaccaaacaagtttgcatagcatggtccagctcaagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgcatcaatgggtcttgtacagtagtaatgactgacggaagtgcctcagggaaggctgacactagaatattatttgtcaaagagggaaagattgttcacattagcccattatcaggaagtgctcagcatatagaggaatgttcctgttatccccgctacccaaacgtcagatgtgtctgcagagacaactggaagggctctaataggcccactatagacataaacatgacagattatagcattgactccagttatgtgtgctcaggactcgttggggatacaccaagaaatgatgatagttctagcagcagcaattgcagggatcctaatgaagagaggggaaaccaaggtgtgaaaggatgggcctttgacagtggagatgatgtttggatgggtagaacaatcagtagagattcgcggtcaggctatgagacatttagggtcattggtggttggaccactaccaattccaagtcacagacaaatagacaagtcatagttgataataacaattggtctggttattctggtattttctctgttgaacataaaagctgtatcaataggtgtttttatgtggagttaataagaggaaggccgaaagaaaccagagtatggtggacctcaaacagtattgttgtgttttgtggcacttcaggcacttatggaacaggctcatggcctgatggggcaaatatcaatttcatgcctatatgagctttcgcaattttagaaaaactccttgtttctact

SEQ ID NO: 104 - DNA, 1565 bp, Influenza virus; identical to SEQ ID NO: 88

SEQ ID NO: 105 - DNA, 890 bp, Influenza virus; identical to SEQ ID NO: 97

SEQ ID NO: 106 - DNA, 2233 bp, Influenza virus
agcgaaagcaggtactgattcaaaatggaagactttgtgcgacagtgcttcaatccaatgattgtcgagcttgcggaaaaggcaatgaaagaatatggggagaatccgaaaatcgaaactaacaaatttgctgcaatatgcactcacctggaagtatgcttcatgtattcggatttccatttcattaacgaacagggcgaatcaataatcgtagagtctggtgatccaaatgcattactgaagcatcgatttgagataattgaagggagagaccgaacaatagcctggacagtagtgaatagtatctgcaacaccacgggggtagagaagcctaagttccttccagatctgtatgattacaaggaaaatcgattcattgaaattggagtgacacgaagggaggttcacatatactatctagagaaagccaacaagataaaatctgagatgacacacattcacattttctcattcactggagaggaaatggccaccaaagcggactacacactagacgaagagagtagggcaagaataaaaaccagactgttcactataagacaagaaatggccagcaggggcttatgggattcctttcgtcagtccgaaagaggcgaagatacaattgaagaaaaatttgaaatcacaggaaccatgcgcaggcttgccgaccaaagtcttccaccgaacttctccagccttgaaaattttagagcctatgtggatggatttaaaccgaacggctgcattgagggaaagctttctcaaatgtcaaaagaagtgaacgcaagaattgaaccatttttgaagacaacaccacgccctctcaaattaccagatggccctccttgctctcaacgatcgaaattcttgctgatggatgccttaaaattaagcattgaagatccgagtcatgagggagaaggaatacctctgtatgatgcaatcaaatgcttgaaaacattttttggctggaaagagcctaacatagtcaagccacatgagaaaggcataaatcccaattacctcctggcttggaaacaggtgctagcagaacttcaagacattgaaaatgatgaaaagatcccaaagacaaagaacatgagaaaaacgagccaattgaagtgggcacttggtgaaaacatggcaccagaaaaggtagacttcgaggactgcaaggatgttagcgatctaaaacagtataatagtgatgagccaaagtctagatctctagcaagttggatccagagtgaattcaataaggcatgtgaattgaccgattcaagttggatagagcttgatgaaataggggaggatattgccccaattgagtacattgcaagtatgaggaggaactattttacagcagaagtgtcccattgtagggctactgaatacataatgaaaggagtgtatataaacacagctttgctcaatgcatcttgtgctgccatggatgattttcagctgatcccaatgataagcaaatgcaggataaaagaagggaggcggaaaacaaacctgtatgggttcattataaaaggaaggtctcatttgagaaatgacactgacgtggtaaactttgtgagcatggaattttctctcactgacccgagactggaaccacacaaatgggagagatactgtgttcttgaaataggagatatgctcctaaggactgcaataggccaggtatcaaggcccatgttcttgtatgttagaaccaatggaacttccaaaattaagatgaagtggggtatggagatgaggcgctgccttcttcaatctcttcagcagattgagagcatgattgaggcagagtcgtctgtcaaagaaaaagacatgacaaaagaattctttgaaaataaatctgagacatggccaattggagagtcacccaagggggtggaagagggctccatcggaaaagtgtgcaggaccttgctggcaaaatctgtattcaacagtctgtatgcatctccacaactcgaggggttctcagctgaatcaaggaaactgcttctcattgttcaggcacttagggacaacctggaacctggaaccttcgatcttggagggctatatgaagcaattgaggagtgcctgattaatgatccctgggttttgcttaatgcatcttggtttaactccttcctcacacatgcactgagatagttgtggcaatgctactgtttgctatccatactgtccaaaaaagtaccttgtttctact

SEQ ID NO: 107 - DNA, 2341 bp, Influenza virus; identical to SEQ ID NO: 91

SEQ ID NO: 108 - DNA, 2341 bp, Influenza virus; identical to SEQ ID NO: 92

SEQ ID NO: 109 - DNA, 1709 bp, Influenza virus; identical to SEQ ID NO: 85

SEQ ID NO: 110 - DNA, 1027 bp, Influenza virus; identical to SEQ ID NO: 86

SEQ ID NO: 111 - DNA, 1418 bp, Influenza virus
agcaaaagcaggagtaaaaaatgaacccaaatcagaagataataacaattggctccgtctctctaatcattgcaacagtatgctttctcatgcagattgccattctagcaatgactgtgacactgcatttcaagcaatatgaatgcagcatttccaataacagtcaggtagtgccacatgaaccaactacagagaaagaggtcttttcgaaaggagtagactataggagttggtcaaagccgcagtgtcaaattacaggatttgctcctttttccaaggacaattcaattcgactttctgctggtggaaacatttggataacaagagagccttatgtgtcgtgtgataccagcaaatgttaccaatttgcacttggacaggggaccacattggataacaaacattcaaacggaacaatacatgatagagtcccccatcggacccttttgatgaatgaactgggtgttccatttcacttgggaaccaaacaagtttgcatagcatggtccagctcaagttgccatgatgggaaagcatggttacacgtttgtgtcactggggaagatagaaatgcgactgctagtttcatttacaatggaatgcttgttgacagtattggttcatggtctcaaaacatcctcagaacccaggagtcagaatgcgtttgcatcaatgggtcttgtacagtagtaatgactgacggaagtgcctcagggaaggctgacactagaatattatttgtcaaagagggaaagattgttcacattagcccattatcaggaagtgctcagcatatagaggaatgttcctgttatccccgctacccaaacgtcagatgtgtctgcagagacaactggaagggctctaataggcccactatagacataaacatgacagattatagcattgactccagttatgtgtgctcaggactcgttggggatacaccaagaaatgatgatagttctagcagcagcaattgcagggatcctaatgaagagaggggaaaccaaggtgtgaaaggatgggcctttgacagtggagatgatgtttggatgggtagaacaatcagtagagattcgcggtcaggctatgagacatttagggtcattggtggttggaccactaccaattccaagtcacagacaaatagacaagtcatagttgataataacaattggtctggttattctggtattttctctgttgaacataaaagctgtatcaataggtgtttttatgtggagttaataagaggaaggccgaaagaaaccagagtatggtggacctcaaacagtattgttgtgttttgtggcacttcaggcacttatggaacaggctcatggcctgatggggcaaatatcaatttcatgcctatatgagctttcgcaattttagaaaaactccttgtttctact

SEQ ID NO: 112 - DNA, 1565 bp, Influenza virus; identical to SEQ ID NO: 88

SEQ ID NO: 113 - DNA, 890 bp, Influenza virus; identical to SEQ ID NO: 97

SEQ ID NO: 114 - DNA, 2233 bp, Influenza virus
agcgaaagcaggtactgattcaaaatggaagactttgtgcgacagtgcttcaatccaatgattgtcgagcttgcggaaaaggcaatgaaagaatatggggagaatccgaaaatcgaaactaacaaatttgctgcaatatgcactcacctggaagtatgcttcatgtattcggatttccatttcattaacgaacagggcgaatcaataatcgtagagtctggtgatccaaatgcattactgaagcatcgatttgagataattgaagggagagaccgaacaatagcctggacagtagtgaatagtatctgcaacaccacgggggtagagaagcctaagttccttccagatctgtatgattacaaggaaaatcgattcattgaaattggagtgacacgaagggaggttcacatatactatctagagaaagccaacaagataaaatctgagatgacacacatccatattttctcattcactggagaggaaatggccaccaaagcggactacacactagacgaagagagtagggcaagaataaaaaccagactgttcactataagacaagaaatggccagcaggggcttatgggattcctttcgtcagtccgaaagaggcgaagatacaattgaagaaaaatttgaaatcacaggaaccatgcgcaggcttgccgaccaaagtcttccaccgaacttctccagccttgaaaattttagagcctatgtggatggatttaaaccgaacggctgcattgagggaaagctttctcaaatgtcaaaagaagtgaacgcaagaattgaaccatttttgaagacaacaccacgccctctcaaattaccagatggccctccttgctctcaacgatcgaaattcttgctgatggatgccttaaaattaagcattgaagatccgagtcatgagggagaaggaatacctctgtatgatgcaatcaaatgcttgaaaacattttttggctggaaagagcctaacatagtcaagccacatgagaaaggcataaatcccaattacctcctggcttggaaacaggtgctagcagaacttcaagacattgaaaatgatgaaaagatcccaaagacaaagaacatgagaaaaacgagccaattgaagtgggcacttggtgaaaacatggcaccagaaaaggtagacttcgaggactgcaaggatgttagcgatctaaaacagtataatagtgatgagccaaagtctagatctctagcaagttggatccagagtgaattcaataaggcatgtgaattgaccgattcaagttggatagagcttgatgaaataggggaggatattgccccaattgagtacattgcaagtatgaggaggaactattttacagcagaagtgtcccattgtagggctactgaatacataatgaaaggagtgtatataaacacagctttgctcaatgcatcttgtgctgccatggatgattttcagctgatcccaatgataagcaaatgcaggataaaagaagggaggcggaaaacaaacctgtatgggttcattataaaaggaaggtctcatttgagaaatgacactgacgtggtaaactttgtgagcatggaattttctctcactgacccgagactggaaccacacaaatgggagagatactgtgttcttgaaataggagatatgctcctaaggactgcaataggccaggtatcaaggcccatgttcttgtatgttagaaccaatggaacttccaaaattaagatgaagtggggtatggagatgaggcgctgccttcttcaatctcttcagcagattgagagcatgattgaggcagagtcgtccgtcaaagaaaaagacatgacaaaagaattctttgaaaataaatctgagacatggccaattggagagtcacccaagggggtggaagagggctccatcggaaaagtgtgcaggaccttgctggcaaaatctgtattcaacagtctgtatgcatctccacaactcgaggggttctcagctgaatcaaggaaactgcttctcattgttcaggcacttagggacaacctggaacctggaaccttcgatcttggagggctatatgaagcaattgaggagtgcctgattaatgatccctgggttttgcttaatgcatcttggtttaactccttcctcacacatgcactgagatagttgtggcaatgctactgtttgctatccatactgtccaaaaaagtaccttgtttctact

SEQ ID NO: 115 - DNA, 2341 bp, Influenza virus; identical to SEQ ID NO: 91

SEQ ID NO: 116 - DNA, 2341 bp, Influenza virus; identical to SEQ ID NO: 92

SEQ ID NO: 117 - DNA, 1709 bp, Influenza virus; identical to SEQ ID NO: 85

SEQ ID NO: 118 - DNA, 1027 bp, Influenza virus; identical to SEQ ID NO: 86

SEQ ID NO: 119 - DNA, 1418 bp, Influenza virus; identical to SEQ ID NO: 87

SEQ ID NO: 120 - DNA, 1565 bp, Influenza virus; identical to SEQ ID NO: 88

SEQ ID NO: 121 - DNA, 890 bp, Influenza virus; identical to SEQ ID NO: 89

SEQ ID NO: 122 - DNA, 2180 bp, Influenza virus
agcgaaagcaggtactgattcaaaatggaagactttgtgcgacagtgcttcaatccaatgattgtcgagcttgcggaaaaggcaatgaaagaatatggggagaatccgaaaatcgaaactaacaaatttgctgcaatatgcactcacctggaagtatgcttcatgtattcggatttccatttcattaacgaacagggcgaatcaataatcgtagagtctggtgatccaaatgcattactgaagcatcgatttgagataattgaagggagagaccgaacaatagcctggacagtagtgaatagtatctgcaacaccacgggggtagagaagcctaagttccttccagatctgtatgattacaaggaaaatcgattcattgaaattggagtgacacgaagggaggttcacatatactatctagagaaagccaacaagataaaatctgagatgacacacattcacattttctcattcactggagaggaaatggccaccaaagcggactacacactagacgaagagagtagggcaagaataaaaaccagactgttcactataagacaagaaatggccagcaggggcttatgggattcctttcgtcagtccgaaagaggcgaagatacaattgaagaaaaatttgaaatcacaggaaccatgcgcaggcttgccgaccaaagtcttccaccgaacttctccagccttgaaaattttagagcctatgtggatggatttaaaccgaacggctgcattgagggaaagctttctcaaatgtcaaaagaagtgaacgcaagaattgaaccatttttgaagacaacaccacgccctctcaaattaccagatggccctccttgctctcaacgatcgaaattcttgctgatggatgccttaaaattaagcattgaagatccgagtcatgagggagaaggaatacctctgtatgatgcaatcaaatgcttgaaaacattttttggctggaaagagcctaacatagtcaagccacatgagaaaggcataaatcccaattacctcctggcttggaaacaggtgctagcagaacttcaagacattgaaaatgatgaaaagatcccaaagacaaagaacatgagaaaaacgagccaattgaagtgggcacttggtgaaaacatggcaccagaaaaggtagacttcgaggactgcaaggatgttagcgatctaaaacagtataatagtgatgagccaaagtctagatctctagcaagttggatccagagtgaattcaataaggcatgtgaattgaccgattcaagttggatagagcttgatgaaataggggaggatattgccccaattgagtacattgcaagtatgaggaggaactattttacagcagaagtgtcccattgtagggctactgaatacataatgaaaggagtgtatataaacacagctttgctcaatgcatcttgtgctgccatggatgattttcagctgatcccaatgataagcaaatgcaggataaaagaagggaggcggaaaacaaacctgtatgggttcattataaaaggaaggtctcatttgagaaatgacactgacgtggtaaactttgtgagcatggaattttctctcactgacccgagactggaaccacacaaatgggagagatactgtgttcttgaaataggagatatgctcctaaggactgcaataggccaggtatcaaggcccatgttcttgtatgttagaaccaatggaacttccaaaattaagatgaagtggggtatggagatgaggcgctgccttcttcaatctcttcagcagattgagagcatgattgaggcagagtcgtccgtcaaagaaaaagacatgacaaaagaattctttgaaaataaatctgagacatggccaattggagagtcacccaagggggtggaagagggctccatcggaaaagtgtgcaggaccttgctggcaaaatctgtattcaacagtctgtatgcatctccacaactcgaggggttctcagctgaatcaaggaaactgcttctcattgttcaggcacttagggacaacctggaacctggaaccttcgatcttggagggctatatgaagcaattgaggagtgcctgattaatgatccctgggttttgcttaatgcatcttggtttaactccttcctcacacatgcactgagatagttgtg

SEQ ID NO: 123 - DNA, 2341 bp, Influenza virus; identical to SEQ ID NO: 91

SEQ ID NO: 124 - DNA, 2341 bp, Influenza virus; identical to SEQ ID NO: 92

SEQ ID NO: 125 - protein, 552 aa, Influenza Virus
MNIQMLVFTACVLTGARGDKICLGHHAVANGTKVNTLTERGVEVVNATETVETVNIKKICTQGKRPTDLGQCGLLGTLIGPPQCDQFLEFSSDLIIERREGNDICYPGKFINEESLRQILRKSGGISKESMGFTYSGIRTNGSTSACTRSGSSFYAEMKWLLSNSDNALFPQMTKSYRNPRNKPALIIWGVHHSGSVNEQIKLYGNGNKLITVRSSKYQQPFTPSPGARRIDFHWLLLDPNDTVTFTFNGAFIAPDRASFFRGESLGIQSDAPLDSSCGGDCFHSGGTIVSSLPFQNINPRIVGKCPRYVKQKSLLLATGMRNVPEKPKPRGLFGAIAGFIENGWEGLINGWYGFRHQNAQGEGTAADYKSTQSAIDQITGKLNRLIGKTNQQFELIDNEFSEIEQQIGNVINWTRDAMTEIWSYNAEMLVAMENQHTIDLTDSEMSKLYERVKKQLRENAEEDGTGCFEIFHKCDNHCMESIRNNTYDHTQYRTESLQNRIRIDPVKLSSGYKDIILWFSFGASCFLLLAIAMGLVFICIKNGNMQCTICI

SEQ ID NO: 126 - protein, 97 aa, Influenza Virus; identical to SEQ ID NO: 81

SEQ ID NO: 127 - protein, 252 aa, Influenza Virus
MSLLTEVETYVLSIIPSGPLKAEIAQRLEDVFAGKNTDLEALMEWLKTRPILSPLTKGILGFVFTLTVPSERGLQRRRFVQNALNGNGDPNNMDRAIKLYRKLKREITFHGAKEVALSYSTGALASCMGLIYNRMGTVTTEGAFGLVCATCEQIADSQHRSHRQMVTTTNPLIRHENRMVLASTTAKAMEQVAGSSEQAAEAMEVASQAKQMVQAMRTIGTHPSSSTGLRDDLLEKLQAYQKRMGVQLHRFK

SEQ ID NO: 128 - protein, 450 aa, Influenza Virus
MNPNQKIITIGSVSLIIATVCFLMQIAILAMTVTLHFKQYECSISNNSQVVPHEPTTEKEVCSKGVDYRSWSKPQCQITGFAPFSKDNSIRLSAGGNIWITREPYVSCDTSKCYQFALGQGTTLDNKHSNGTIHDRVPHRTLLMNELGVPFHLGTKQVCIAWSSSSCHDGKAWLHVCVTGEDRNATASFIYNGMLVDSIGSWSQNILRTQESECVCINGSCTVVMTDGSASGKADTRILFVKEGKIVHISPLSGSAQHIEECSCYPRYPNVRCVCRDNWKGSNRPTIDINMTDYSIDSSYVCSGLVGDTPRNDDSSSSSNCRDPNEERGNQGVKGWAFDSGDDVWMGRTISRDSRSGYETFRVIGGWTTTNSKSQTNRQVIVDNNNWSGYSGIFSVEHKSCINRCFYVELIRGRPKETRVWWTSNSIVVFCGTSGTYGTGSWPDGANINF

SEQ ID NO: 129 - protein, 498 aa, Influenza Virus; identical to SEQ ID NO: 79

SEQ ID NO: 130 - protein, 121 aa, Influenza Virus
MDSNTITSFQEVLQRMSKMQLESSSVDLNGMITQFERLKIYRDSLGESVMRIGDLHSLQNRNATWREELSQKFDEIRWLIAECRNILTKTENSFEQITFLQALQLLLEVESEIRTFSFQLI

SEQ ID NO: 131 - protein, 230 aa, Influenza Virus
MDSNTITSFQVDCYLWHIRKLLSMRDMCDAPFDDRLRRDQKALKGRSSTLGLDLRVATMEGKKIVEDIIKSETDEKLRIAIASSPAPRYITDMSIEEISREWYMLMPRQKMTGGLMVKMDQAIMNKRIMLKANFSVLFDQLETLVSLRAFTDDGAIVAEICPIPSMPGSSAEDVKDAIGILIGGLEWNDNSVRTSENIQRFAWGVRDENRGPPLSSKQKRYMERRVESEI

SEQ ID NO: 132 - protein, 716 aa, Influenza Virus
MEDFVRQCFNPMIVELAEKAMKEYGENPKIETNKFAAICTHLEVCFMYSDFHFINEQGESIIVESGDPNALLKHRFEIIEGRDRTIAWTVVNSICNTTGVEKPKFLPDLYDYKENRFIEIGVTRREVHIYYLEKANKIKSEMTHIHIFSFTGEEMATKADYTLDEESRARIKTRLFTIRQEMASRGLWDSFRQSERGEDTIEEKFEITGTMRRLADQSLPPNFSSLENFRAYVDGFKPNGCIEGKLSQMSKEVNARIEPFLKTTPRPLKLPDGPPCSQRSKFLLMDALKLSIEDPSHEGEGIPLYDAIKCLKTFFGWKEPNIVKPHEKGINPNYLLAWKQVLAELQDIENDEKIPKTKNMRKTSQLKWALGENMAPEKVDFEDCKDVSDLKQYNSDEPKSRSLASWIQSEFNKACELTDSSWIELDEIGEDIAPIEYIASMRRNYFTAEVSHCRATEYIMKGVYINTALLNASCAAMDDFQLIPMISKCRIKEGRRKTNLYGFIIKGRSHLRNDTDVVNFVSMEFSLTDPRLEPHKWERYCVLEIGDMLLRTAIGQVSRPMFLYVRTNGTSKIKMKWGMEMRRCLLQSLQQIESMIEAESSVKEKDMTKEFFENKSETWPIGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 133 - protein, 252 aa, Influenza virus
MEDFVRQCFNPMIVELAEKAMKEYGENPKIETNKFAAICTHLEVCFMYSDFHFINEQGESIIVESGDPNALLKHRFEIIEGRDRTIAWTVVNSICNTTGVEKPKFLPDLYDYKENRFIEIGVTRREVHIYYLEKANKIKSEMTHIHIFSFTGEEMATKADYTLDEESRARIKTRLFTIRQEMASRGLWDSFVSPKEAKIQLKKNLKSQEPCAGLPTKVFHRTSPALKILEPMWMDLNRTAALRESFLKCQKK

SEQ ID NO: 134 - protein, 757 aa, Influenza virus; identical to SEQ ID NO: 74

SEQ ID NO: 135 - protein, 90 aa, Influenza virus; identical to SEQ ID NO: 75

SEQ ID NO: 136 - protein, 759 aa, Influenza virus; identical to SEQ ID NO: 73

SEQ ID NO: 137 - protein, 552 aa, Influenza virus; identical to SEQ ID NO: 125

SEQ ID NO: 138 - protein, 97 aa, Influenza virus; identical to SEQ ID NO: 81

SEQ ID NO: 139 - protein, 252 aa, Influenza virus; identical to SEQ ID NO: 127

SEQ ID NO: 140 - protein, 453 aa, Influenza virus; identical to SEQ ID NO: 80

SEQ ID NO: 141 - protein, 453 aa, Influenza virus; identical to SEQ ID NO: 80

SEQ ID NO: 142 - protein, 121 aa, Influenza virus
MDSNTITSFQEVLQRMSKMQLESSSVDLNGMITQFERLKIYRDSLGESVMRIGDLHSLQNRNATWREELSQKFEEIRWLIAECRNILTKTENSFEQITFLQALQLLLEVESEIRTFSFQLI

SEQ ID NO: 143 - protein, 230 aa, Influenza virus; identical to SEQ ID NO: 131

SEQ ID NO: 144 - protein, 716 aa, Influenza virus; identical to SEQ ID NO: 76

SEQ ID NO: 145 - protein, 252 aa, Influenza virus; identical to SEQ ID NO: 77

SEQ ID NO: 146 - protein, 757 aa, Influenza virus; identical to SEQ ID NO: 74

SEQ ID NO: 147 - protein, 90 aa, Influenza virus
MERGQDTQWTQSVEPINTQKRGSGQQTQKLEHPNLTQLMDHYPRIMSQVDMHKQTVSLKQWLSLKSPTLESLKTHVLRQWKLYNKQEWTN

SEQ ID NO: 148 - protein, 759 aa, Influenza virus
MERIKELRDLMSQSRTREILTKTTVDHMAIIKKYTSGRQEKNPALRMKWMMAMKYPITADKRIMEMVPERNEQGQTLWSKTNDAGSDRVMVSPLAVTWWNRNGPTTSTIHYPKVYKTYFEKVERLKHGTFGPVHFRNQVKIRRRVDINPGHADLSAKEAQNVIMEVVFPNEVGARILTSESQLTITKEKKEELQDCKIAPLMVAYMLERELVRKTRFLPVAGGTSSVYIEVLHLTQGTCWEQMYTPGGGVRNDDVDQSLVIAARNIVRRATVSADPLASLLEMCHSTQIGGIKMVDILRQNPTEEQAVDICRAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGIEQIDSVMGMIGILPDMTPNTEVSLRGIRVSKMGVDEYSSTERVVVSIDRFLRVRDPRGNILLSPEEVSETQGTEKLTITYSSSMMWEINGPESVLVNTYQWIIRNWETVKIQWSQDPTMLYNKMEFEPFQSLVPKAARGQYSGFVRTLFQQMRDVLGTFDTFQIIKLLPFAAAPPEQSRMQFSSLTVNVRGSGMRILVRGNSPVFNYNKATKRLTILGKDAGALTNDPDEGSAGVESAVLRGFLILGKEDRRYGPALSINELSNLAKGEKANVLIGQGDVVLVMKRKRDSSILTDSQTATKRIRMAIN

SEQ ID NO: 149 - protein, 552 aa, Influenza virus; identical to SEQ ID NO: 125

SEQ ID NO: 150 - protein, 97 aa, Influenza virus; identical to SEQ ID NO: 81

SEQ ID NO: 151 - protein, 252 aa, Influenza virus; identical to SEQ ID NO: 127

SEQ ID NO: 152 - protein, 453 aa, Influenza virus
MNPNQKIITIGSVSLIIATVCFLMQIAILAMTVTLHFKQYECSISNNSQVVPHEPTTEKEVCSKGVDYRSWSKPQCQITGFAPFSKDNSIRLSAGGNIWITREPYVSCDTSKCYQFALGQGTTLDNKHSNGTIHDRVPHRTLLMNELGVPFHLGTKQVCIAWSSSSCHDGKAWLHVCVTGEDRNATASFIYNGMLVDSIGSWSQNILRTQESECVCINGSCTVVMTDGSASGKADTRILFVKEGKIVHISPLSGSAQHIEECSCYPRYPNVRCVCRDNWKGSNRPTIDINMTDYSIDSSYVCSGLVGDTPRNDDSSSSSNCRDPNEERGNQGVKGWAFDSGDDVWMGRTISRDSRSGYETFRVIGGWTTTNSKSQTNRQVIVDNNNWSGYSGIFSVEHKSCINRCFYVELIRGRPKETRVWWTSNSIVVFCGTSGTYGTGSWPDGANINFMPI

SEQ ID NO: 153 - protein, 498 aa, Influenza virus; identical to SEQ ID NO: 79

SEQ ID NO: 154 - protein, 121 aa, Influenza virus; identical to SEQ ID NO: 142

SEQ ID NO: 155 - protein, 230 aa, Influenza virus; identical to SEQ ID NO: 131

SEQ ID NO: 156 - protein, 716 aa, Influenza virus; identical to SEQ ID NO: 132

SEQ ID NO: 157 - protein, 252 aa, Influenza virus; identical to SEQ ID NO: 133

SEQ ID NO: 158 - protein, 757 aa, Influenza virus; identical to SEQ ID NO: 74

SEQ ID NO: 159 - protein, 90 aa, Influenza virus; identical to SEQ ID NO: 75

SEQ ID NO: 160 - protein, 759 aa, Influenza virus; identical to SEQ ID NO: 73

SEQ ID NO: 161 - protein, 552 aa, Influenza virus; identical to SEQ ID NO: 125

SEQ ID NO: 162 - protein, 97 aa, Influenza virus; identical to SEQ ID NO: 81

SEQ ID NO: 163 - protein, 252 aa, Influenza virus; identical to SEQ ID NO: 127

SEQ ID NO: 164 - protein, 453 aa, Influenza virus
MNPNQKIITIGSVSLIIATVCFLMQIAILAMTVTLHFKQYECSISNNSQVVPHEPTTEKEVFSKGVDYRSWSKPQCQITGFAPFSKDNSIRLSAGGNIWITREPYVSCDTSKCYQFALGQGTTLDNKHSNGTIHDRVPHRTLLMNELGVPFHLGTKQVCIAWSSSSCHDGKAWLHVCVTGEDRNATASFIYNGMLVDSIGSWSQNILRTQESECVCINGSCTVVMTDGSASGKADTRILFVKEGKIVHISPLSGSAQHIEECSCYPRYPNVRCVCRDNWKGSNRPTIDINMTDYSIDSSYVCSGLVGDTPRNDDSSSSSNCRDPNEERGNQGVKGWAFDSGDDVWMGRTISRDSRSGYETFRVIGGWTTTNSKSQTNRQVIVDNNNWSGYSGIFSVEHKSCINRCFYVELIRGRPKETRVWWTSNSIVVFCGTSGTYGTGSWPDGANINFMPI

SEQ ID NO: 165 - protein, 498 aa, Influenza virus; identical to SEQ ID NO: 79

SEQ ID NO: 166 - protein, 121 aa, Influenza virus; identical to SEQ ID NO: 142

SEQ ID NO: 167 - protein, 230 aa, Influenza virus; identical to SEQ ID NO: 131

SEQ ID NO: 168 - protein, 716 aa, Influenza virus; identical to SEQ ID NO: 132

SEQ ID NO: 169 - protein, 252 aa, Influenza virus; identical to SEQ ID NO: 133

SEQ ID NO: 170 - protein, 757 aa, Influenza virus; identical to SEQ ID NO: 74

SEQ ID NO: 171 - protein, 90 aa, Influenza virus; identical to SEQ ID NO: 75

SEQ ID NO: 172 - protein, 759 aa, Influenza virus; identical to SEQ ID NO: 73

SEQ ID NO: 173 - protein, 552 aa, Influenza virus; identical to SEQ ID NO: 125

SEQ ID NO: 174 - protein, 97 aa, Influenza virus; identical to SEQ ID NO: 81

SEQ ID NO: 175 - protein, 252 aa, Influenza virus; identical to SEQ ID NO: 127

SEQ ID NO: 176 - protein, 453 aa, Influenza virus; identical to SEQ ID NO: 152

SEQ ID NO: 177 - protein, 498 aa, Influenza virus; identical to SEQ ID NO: 79

SEQ ID NO: 178 - protein, 121 aa, Influenza virus; identical to SEQ ID NO: 130

SEQ ID NO: 179 - protein, 230 aa, Influenza virus; identical to SEQ ID NO: 131

SEQ ID NO: 180 - protein, 716 aa, Influenza virus; identical to SEQ ID NO: 132

SEQ ID NO: 181 - protein, 252 aa, Influenza virus; identical to SEQ ID NO: 133

SEQ ID NO: 182 - protein, 757 aa, Influenza virus; identical to SEQ ID NO: 74

SEQ ID NO: 183 - protein, 90 aa, Influenza virus; identical to SEQ ID NO: 75

SEQ ID NO: 184 - protein, 715 aa, Influenza virus
LRMKWMMAMKYPITADKRIMEMVPERNEQGQTLWSKTNDAGSDRVMVSPLAVTWWNRNGPTTSTIHYPKVYKTYFEKVERLKHGTFGPVHFRNQVKIRRRVDINPGHADLSAKEAQNVIMEVVFPNEVGARILTSESQLTITKEKKEELQDCKIAPLMVAYMLERELVRKTRFLPVAGGTSSVYIEVLHLTQGTCWEQMYTPGGGVRNDDVDQSLVIAARNIVRRATVSADPLASLLEMCHSTQIGGIKMVDILRQNPTEEQAVDICRAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQSWGIEQIDSVMGMIGILPDMTPNTEVSLRGIRVSKMGVDEYSSTERVVVSIDRFLRVRDPRGNILLSPEEVSETQGTEKLTITYSSSMMWEINGPESVLVNTYQWIIRNWETVKIQWSQDPTMLYNKMEFEPFQSLVPKAARGQYSGFVRTLFQQMRDVLGTFDTFQIIKLLPFAAAPPEQSRMQFSSLTVNVRGSGMRILVRGNSPVFNYNKATKRLTILGKDAGALTNDPDEGSAGVESAVLRGFLILGKEDRRYGPALSINELSNLAKGEKANVLIGQGDVVLVMKRKRDSSILTDSQTATKRIRMAIN